SEQUENCE LISTING (152 sequences)

SEQ ID NO: 1 - protein, 19 aa, Homo sapiens
PLSQETFSDLWKLLPENNV

SEQ ID NO: 2 - protein, 25 aa, Homo sapiens
QEDIIRNIARHLAQVGDSMDRSIPP

SEQ ID NO: 3 - protein, 25 aa, Homo sapiens
DNRPEIWIAQELRRIGDEFNAYYAR

SEQ ID NO: 4 - protein, 25 aa, Homo sapiens
NLWAAQRYGRELRRMSDEFVDSFKK

SEQ ID NO: 5 - protein, 25 aa, Homo sapiens
EEQWAREIGAQLRRMADDLNAQYER

SEQ ID NO: 6 - protein, 24 aa, Homo sapiens
RSSAAQLTAARLKALGDELHQRTM

SEQ ID NO: 7 - protein, 22 aa, Homo sapiens
AELPPEFAAQLRKIGDKVYCTW

SEQ ID NO: 8 - protein, 25 aa, Homo sapiens
VPADLKDECAQLRRIGDKVNLRQKL

SEQ ID NO: 9 - protein, 24 aa, Homo sapiens
QHRAEVQIARKLQCIADQFHRLHT

SEQ ID NO: 10 - protein, 22 aa, Homo sapiens
SSAAQLTAARLKALGDELHQRT

SEQ ID NO: 11 - protein, 25 aa, Homo sapiens
CMEGSDALALRLACIGDEMDVSLRA

SEQ ID NO: 12 - protein, 24 aa, Homo sapiens
DIERRKEVESILKKNSDWIWDWSS

SEQ ID NO: 13 - protein, 22 aa, Homo sapiens
GRLAEVCAVLLRLGDELEMIRP

SEQ ID NO: 14 - protein, 25 aa, Homo sapiens
PQDASTKKSECLKRIGDELDSNMEL

SEQ ID NO: 15 - protein, 22 aa, Homo sapiens
PSSTMGQVGRQLAIIGDDINRR

SEQ ID NO: 16 - protein, 14 aa, Homo sapiens
KQALREAGDEFELR

SEQ ID NO: 17 - protein, 22 aa, Homo sapiens
LSPPVVHLALALRQAGDDFSRR

SEQ ID NO: 18 - protein, 23 aa, Homo sapiens
EVIPMAAVKQALREAGDEFELRY

SEQ ID NO: 19 - protein, 20 aa, Homo sapiens
PADPLHQAMRAAGDEFETRF

SEQ ID NO: 20 - protein, 23 aa, Homo sapiens
ATSRKLETLRRVGDGVQRNHETA

SEQ ID NO: 21 - protein, 19 aa, Homo sapiens
LAEVCTVLLRLGDELEQIR

SEQ ID NO: 22 - protein, 19 aa, Homo sapiens
MTVGELSRALGHENGSLDP

SEQ ID NO: 23 - protein, 22 aa, Homo sapiens
VVEGEKEVEALKKSADWVSDWS

SEQ ID NO: 24 - protein, 20 aa, Homo sapiens
SMARDPQRYLVIQGDDRMKL

SEQ ID NO: 25 - protein, 25 aa, Homo sapiens
  MOD_RES (14)..(14): Cross linked amino acid residue
QEDIIRNIARHLAXVGDXMDRSIPP

SEQ ID NO: 26 - protein, 25 aa, Homo sapiens
  MOD_RES (14)..(14): Cross linked amino acid residue
DNRPEIWIAQELRXIGDXFNAYYAR

SEQ ID NO: 27 - protein, 25 aa, Homo sapiens
  MOD_RES (14)..(14): Cross linked amino acid residue
NLWAAQRYGRELRXMSDXFVDSFKK

SEQ ID NO: 28 - protein, 25 aa, Homo sapiens
  MOD_RES (14)..(14): Cross linked amino acid residue
EEQWAREIGAQLRXMADXLNAQYER

SEQ ID NO: 29 - protein, 24 aa, Homo sapiens
  MOD_RES (14)..(14): Cross linked amino acid residue
RSSAAQLTAARLKXLGDXLHQRTM

SEQ ID NO: 30 - protein, 22 aa, Homo sapiens
  MOD_RES (13)..(13): Cross linked amino acid residue
AELPPEFAAQLRXIGDXVYCTW

SEQ ID NO: 31 - protein, 25 aa, Homo sapiens
  MOD_RES (14)..(14): Cross linked amino acid residue
VPADLKDECAQLRXIGDXVNLRQKL

SEQ ID NO: 32 - protein, 24 aa, Homo sapiens
  MOD_RES (14)..(14): Cross linked amino acid residue
QHRAEVQIARKLQXIADXFHRLHT

SEQ ID NO: 33 - protein, 22 aa, Homo sapiens
  MOD_RES (13)..(13): Cross linked amino acid residue
SSAAQLTAARLKXLGDXLHQRT

SEQ ID NO: 34 - protein, 25 aa, Homo sapiens
  MOD_RES (14)..(14): Cross linked amino acid residue
CMEGSDALALRLAXIGDXMDVSLRA

SEQ ID NO: 35 - protein, 24 aa, Homo sapiens
  MOD_RES (14)..(14): Cross linked amino acid residue
DIERRKEVESILKXNSDXIWDWSS

SEQ ID NO: 36 - protein, 22 aa, Homo sapiens
  MOD_RES (12)..(12): Cross linked amino acid residue
GRLAEVCAVLLXLGDXLEMIRP

SEQ ID NO: 37 - protein, 25 aa, Homo sapiens
  MOD_RES (14)..(14): Cross linked amino acid residue
PQDASTKKSECLKXIGDXLDSNMEL

SEQ ID NO: 38 - protein, 22 aa, Homo sapiens
  MOD_RES (14)..(14): Cross linked amino acid residue
PSSTMGQVGRQLAXIGDXINRR

SEQ ID NO: 39 - protein, 14 aa, Homo sapiens
  MOD_RES (6)..(6): Cross linked amino acid residue
KQALRXAGDXFELR

SEQ ID NO: 40 - protein, 22 aa, Homo sapiens
  MOD_RES (14)..(14): Cross linked amino acid residue
LSPPVVHLALALRXAGDXFSRR

SEQ ID NO: 41 - protein, 23 aa, Homo sapiens
  MOD_RES (14)..(14): Cross linked amino acid residue
EVIPMAAVKQALRXAGDXFELRY

SEQ ID NO: 42 - protein, 20 aa, Homo sapiens
  MOD_RES (11)..(11): Cross linked amino acid residue
PADPLHQAMRXAGDXFETRF

SEQ ID NO: 43 - protein, 23 aa, Homo sapiens
  MOD_RES (11)..(11): Cross linked amino acid residue
ATSRKLETLRXVGDXVQRNHETA

SEQ ID NO: 44 - protein, 19 aa, Homo sapiens
  MOD_RES (10)..(10): Cross linked amino acid residue
LAEVCTVLLXLGDXLEQIR

SEQ ID NO: 45 - protein, 19 aa, Homo sapiens
  MOD_RES (12)..(12): Cross linked amino acid residue
MTVGELSRALGXENGXLDP

SEQ ID NO: 46 - protein, 22 aa, Homo sapiens
  MOD_RES (13)..(13): Cross linked amino acid residue
VVEGEKEVEALKXSADXVSDWS

SEQ ID NO: 47 - protein, 20 aa, Homo sapiens
  MOD_RES (12)..(12): Cross linked amino acid residue
SMARDPQRYLVXQGDXRMKL

SEQ ID NO: 48 - protein, 25 aa, Homo sapiens
  MOD_RES (9)..(9): Cross linked amino acid residue
QEDIIRNIXRHLXQVGDSMDRSIPP

SEQ ID NO: 49 - protein, 25 aa, Homo sapiens
  MOD_RES (9)..(9): Cross linked amino acid residue
DNRPEIWIXQELXRIGDEFNAYYAR

SEQ ID NO: 50 - protein, 25 aa, Homo sapiens
  MOD_RES (9)..(9): Cross linked amino acid residue
NLWAAQRYXRELXRMSDEFVDSFKK

SEQ ID NO: 51 - protein, 25 aa, Homo sapiens
  MOD_RES (9)..(9): Cross linked amino acid residue
EEQWAREIXAQLXRMADDLNAQYER

SEQ ID NO: 52 - protein, 24 aa, Homo sapiens
  MOD_RES (9)..(9): Cross linked amino acid residue
RSSAAQLTXARLXALGDELHQRTM

SEQ ID NO: 53 - protein, 22 aa, Homo sapiens
  MOD_RES (8)..(8): Cross linked amino acid residue
AELPPEFXAQLXKIGDKVYCTW

SEQ ID NO: 54 - protein, 25 aa, Homo sapiens
  MOD_RES (9)..(9): Cross linked amino acid residue
VPADLKDEXAQLXRIGDKVNLRQKL

SEQ ID NO: 55 - protein, 24 aa, Homo sapiens
  MOD_RES (9)..(9): Cross linked amino acid residue
QHRAEVQIXRKLXCIADQFHRLHT

SEQ ID NO: 56 - protein, 22 aa, Homo sapiens
  MOD_RES (8)..(8): Cross linked amino acid residue
SSAAQLTXARLXALGDELHQRT

SEQ ID NO: 57 - protein, 25 aa, Homo sapiens
  MOD_RES (9)..(9): Cross linked amino acid residue
CMEGSDALXLRLXCIGDEMDVSLRA

SEQ ID NO: 58 - protein, 24 aa, Homo sapiens
  MOD_RES (9)..(9): Cross linked amino acid residue
DIERRKEVXSILXKNSDWIWDWSS

SEQ ID NO: 59 - protein, 22 aa, Homo sapiens
  MOD_RES (7)..(7): Cross linked amino acid residue
GRLAEVXAVLXRLGDELEMIRP

SEQ ID NO: 60 - protein, 25 aa, Homo sapiens
  MOD_RES (9)..(9): Cross linked amino acid residue
PQDASTKKXECLXRIGDELDSNMEL

SEQ ID NO: 61 - protein, 22 aa, Homo sapiens
  MOD_RES (9)..(9): Cross linked amino acid residue
PSSTMGQVXRQLXIIGDDINRR

SEQ ID NO: 62 - protein, 14 aa, Homo sapiens
  MOD_RES (1)..(1): Cross linked amino acid residue
XQALXEAGDEFELR

SEQ ID NO: 63 - protein, 22 aa, Homo sapiens
  MOD_RES (9)..(9): Cross linked amino acid residue
LSPPVVHLXLALXQAGDDFSRR

SEQ ID NO: 64 - protein, 23 aa, Homo sapiens
  MOD_RES (9)..(9): Cross linked amino acid residue
EVIPMAAVXQALXEAGDEFELRY

SEQ ID NO: 65 - protein, 20 aa, Homo sapiens
  MOD_RES (6)..(6): Cross linked amino acid residue
PADPLXQAMXAAGDEFETRF

SEQ ID NO: 66 - protein, 23 aa, Homo sapiens
  MOD_RES (6)..(6): Cross linked amino acid residue
ATSRKXETLXRVGDGVQRNHETA

SEQ ID NO: 67 - protein, 19 aa, Homo sapiens
  MOD_RES (5)..(5): Cross linked amino acid residue
LAEVXTVLXRLGDELEQIR

SEQ ID NO: 68 - protein, 19 aa, Homo sapiens
  MOD_RES (7)..(7): Cross linked amino acid residue
MTVGELXRALXHENGSLDP

SEQ ID NO: 69 - protein, 22 aa, Homo sapiens
  MOD_RES (8)..(8): Cross linked amino acid residue
VVEGEKEXEALXKSADWVSDWS

SEQ ID NO: 70 - protein, 20 aa, Homo sapiens
  MOD_RES (7)..(7): Cross linked amino acid residue
SMARDPXRYLXIQGDDRMKL

SEQ ID NO: 71 - protein, 16 aa, Homo sapiens
LSQETFSDLWKLLPEN

SEQ ID NO: 72 - protein, 16 aa, Homo sapiens
  MOD_RES (9)..(9): Cross linked amino acid residue
LSQETFSDXWKLLPEX

SEQ ID NO: 73 - protein, 16 aa, Homo sapiens
  MOD_RES (5)..(5): Cross linked amino acid residue
LSQEXFSDLWKXLPEN

SEQ ID NO: 74 - protein, 16 aa, Homo sapiens
  MOD_RES (4)..(4): Cross linked amino acid residue
LSQXTFSDLWXLLPEN

SEQ ID NO: 75 - protein, 16 aa, Homo sapiens
  MOD_RES (7)..(7): Cross linked amino acid residue
LSQETFXDLWKLLXEN

SEQ ID NO: 76 - protein, 16 aa, Homo sapiens
  MOD_RES (7)..(7): Cross linked amino acid residue
QSQQTFXNLWRLLXQN

SEQ ID NO: 77 - protein, 8 aa, Homo sapiens
DRVYIHPF

SEQ ID NO: 78 - protein, 14 aa, Homo sapiens
EQRLGNQWAVGHLM

SEQ ID NO: 79 - protein, 9 aa, Homo sapiens
RPPGFSPFR

SEQ ID NO: 80 - protein, 10 aa, Homo sapiens
ISHKDMQLGR

SEQ ID NO: 81 - protein, 10 aa, Homo sapiens
ARASHLGLAR

SEQ ID NO: 82 - protein, 13 aa, Homo sapiens
SYSMEHFRWGKPV

SEQ ID NO: 83 - protein, 8 aa, Homo sapiens
  MOD_RES (3)..(3): Cross linked amino acid residue
DRXYXHPF

SEQ ID NO: 84 - protein, 14 aa, Homo sapiens
  MOD_RES (7)..(7): Cross linked amino acid residue
EQRLGNXWAVGHLX

SEQ ID NO: 85 - protein, 10 aa, Homo sapiens
  MOD_RES (4)..(4): Cross linked amino acid residue
RPPXFSPFRX

SEQ ID NO: 86 - protein, 11 aa, Homo sapiens
  MOD_RES (7)..(7): Cross linked amino acid residue
ISHKDMXLGRX

SEQ ID NO: 87 - protein, 11 aa, Homo sapiens
  MOD_RES (7)..(7): Cross linked amino acid residue
ARASHLXLARX

SEQ ID NO: 88 - protein, 13 aa, Homo sapiens
  MOD_RES (5)..(5): Cross linked amino acid residue
SYSMXHFRWXKPV

SEQ ID NO: 89 - protein, 21 aa, Artificial Sequence
  Description of Artificial Sequence Synthetic peptide
DIIRNIARHLAXVGDXXDRSI

SEQ ID NO: 90 - protein, 21 aa, Artificial Sequence
  Description of Artificial Sequence Synthetic peptide
IWIAQELRXIGDXFNAYYARR

SEQ ID NO: 91 - protein, 16 aa, Artificial Sequence
  Description of Artificial Sequence Synthetic peptide
QSQQTFXNLWRLLXQN

SEQ ID NO: 92 - protein, 16 aa, Artificial Sequence
  Description of Artificial Sequence Synthetic peptide
QSQQTFXNLWRLLXQN

SEQ ID NO: 93 - protein, 16 aa, Artificial Sequence
  Description of Artificial Sequence Synthetic peptide
QSQQTFXNLWRLLXQN

SEQ ID NO: 94 - protein, 16 aa, Artificial Sequence
  Description of Artificial Sequence Synthetic peptide
QSQQTFXNLWRLLXQN

SEQ ID NO: 95 - protein, 21 aa, Artificial Sequence
  Description of Artificial Sequence Synthetic peptide
IWIAQELRXIGDXFNAYYARR

SEQ ID NO: 96 - protein, 21 aa, Artificial Sequence
  Description of Artificial Sequence Synthetic peptide
IWIAQELRXIGDXFNAYYARR

SEQ ID NO: 97 - protein, 21 aa, Artificial Sequence
  Description of Artificial Sequence Synthetic peptide
DIIRNIARHLAXVGDXXDRSI

SEQ ID NO: 98 - protein, 21 aa, Artificial Sequence
  Description of Artificial Sequence Synthetic peptide
DIIRNIARHLAXVGDXXDRSI

SEQ ID NO: 99 - protein, 21 aa, Artificial Sequence
  Description of Artificial Sequence Synthetic peptide
DIIRNIARHLAXVGDXXDRSI

SEQ ID NO: 100 - protein, 21 aa, Artificial Sequence
  Description of Artificial Sequence Synthetic peptide
DIIRNIARHLAXVGDXXDRSI

SEQ ID NO: 101 - protein, 21 aa, Artificial Sequence
  Description of Artificial Sequence Synthetic peptide
DIIRNIARHLAXVGDXXDRSI

SEQ ID NO: 102 - protein, 21 aa, Artificial Sequence
  Description of Artificial Sequence Synthetic peptide
DIIRNIARHLAXVGDXXDRSI

SEQ ID NO: 103 - protein, 21 aa, Artificial Sequence
  Description of Artificial Sequence Synthetic peptide
DIIRNIARHLAXVGDXXDRSI

SEQ ID NO: 104 - protein, 21 aa, Artificial Sequence
  Description of Artificial Sequence Synthetic peptide
DIIRNIARHLAXVGDXXDRSI

SEQ ID NO: 105 - protein, 21 aa, Artificial Sequence
  Description of Artificial Sequence Synthetic peptide
DIIRNIARHLAXVGDXXDRSI

SEQ ID NO: 106 - protein, 21 aa, Artificial Sequence
  Description of Artificial Sequence Synthetic peptide
DIIRNIARHLAXVGDXXDRSI

SEQ ID NO: 107 - protein, 21 aa, Artificial Sequence
  Description of Artificial Sequence Synthetic peptide
DIIRNIARHLAXVGDXXDRSI

SEQ ID NO: 108 - protein, 21 aa, Artificial Sequence
  Description of Artificial Sequence Synthetic peptide
DIIRNIARHLAXVGDXXDRSI

SEQ ID NO: 109 - protein, 21 aa, Artificial Sequence
  Description of Artificial Sequence Synthetic peptide
DIIRNIARHLAXVGDXXDRSI

SEQ ID NO: 110 - protein, 21 aa, Artificial Sequence
  Description of Artificial Sequence Synthetic peptide
DIIRNIARHLAXVGDXXDRSI

SEQ ID NO: 111 - protein, 21 aa, Artificial Sequence
  Description of Artificial Sequence Synthetic peptide
DIIRNIARHLAXVGDXXDRSI

SEQ ID NO: 112 - protein, 21 aa, Artificial Sequence
  Description of Artificial Sequence Synthetic peptide
DIIRNIARHLAXVGDXXDRSI

SEQ ID NO: 113 - protein, 21 aa, Artificial Sequence
  Description of Artificial Sequence Synthetic peptide
DIIRNIARHLAXVGDXXDRSI

SEQ ID NO: 114 - protein, 21 aa, Artificial Sequence
  Description of Artificial Sequence Synthetic peptide
DIIRNIARHLAXVGDXXDRSI

SEQ ID NO: 115 - protein, 21 aa, Artificial Sequence
  Description of Artificial Sequence Synthetic peptide
IWIAQELRXIGDXFNAYYARR

SEQ ID NO: 116 - protein, 21 aa, Artificial Sequence
  Description of Artificial Sequence Synthetic peptide
IWIAQELRXIGDXFNAYYARR

SEQ ID NO: 117 - protein, 21 aa, Artificial Sequence
  Description of Artificial Sequence Synthetic peptide
IWIAQELRSIGDSFNAYYARR

SEQ ID NO: 118 - protein, 21 aa, Artificial Sequence
  Description of Artificial Sequence Synthetic peptide
IWIAQELRSIGDSFNAYYARR

SEQ ID NO: 119 - protein, 21 aa, Artificial Sequence
  Description of Artificial Sequence Synthetic peptide
IWIAQELRCIGDCFNAYYARR

SEQ ID NO: 120 - protein, 21 aa, Artificial Sequence
  Description of Artificial Sequence Synthetic peptide
IWIAQELRCIGDCFNAYYARR

SEQ ID NO: 121 - protein, 21 aa, Artificial Sequence
  Description of Artificial Sequence Synthetic peptide
IWIAQELRXIGDXFNAYYARR

SEQ ID NO: 122 - protein, 21 aa, Artificial Sequence
  Description of Artificial Sequence Synthetic peptide
IWIAQELRXIGDXFNAYYARR

SEQ ID NO: 123 - protein, 21 aa, Artificial Sequence
  Description of Artificial Sequence Synthetic peptide
DIIRNIARHLACVGDCXDRSI

SEQ ID NO: 124 - protein, 21 aa, Artificial Sequence
  Description of Artificial Sequence Synthetic peptide
DIIRNIARHLACVGDCXDRSI

SEQ ID NO: 125 - protein, 21 aa, Artificial Sequence
  Description of Artificial Sequence Synthetic peptide
DIIRNIARHLAXVGDXXDRSI

SEQ ID NO: 126 - protein, 21 aa, Artificial Sequence
  Description of Artificial Sequence Synthetic peptide
DIIRNIARHLAXVGDXXDRSI

SEQ ID NO: 127 - protein, 21 aa, Artificial Sequence
  Description of Artificial Sequence Synthetic peptide
DIIRNIARHLACVGDCXDRSI

SEQ ID NO: 128 - protein, 21 aa, Artificial Sequence
  Description of Artificial Sequence Synthetic peptide
DIIRNIARHLACVGDCXDRSI

SEQ ID NO: 129 - protein, 21 aa, Artificial Sequence
  Description of Artificial Sequence Synthetic peptide
DIIRNIARHLASVGDSXDRSI

SEQ ID NO: 130 - protein, 21 aa, Artificial Sequence
  Description of Artificial Sequence Synthetic peptide
DIIRNIARHLASVGDSXDRSI

SEQ ID NO: 131 - protein, 21 aa, Artificial Sequence
  Description of Artificial Sequence Synthetic peptide
IWIAQELRCIGDCFNAYYARR

SEQ ID NO: 132 - protein, 21 aa, Artificial Sequence
  Description of Artificial Sequence Synthetic peptide
IWIAQELRCIGDCFNAYYARR

SEQ ID NO: 133 - protein, 16 aa, Artificial Sequence
  Description of Artificial Sequence Synthetic peptide
QSQQTFXNLWRLLXQN

SEQ ID NO: 134 - protein, 16 aa, Artificial Sequence
  Description of Artificial Sequence Synthetic peptide
QSQQTFXNLWRLLXQN

SEQ ID NO: 135 - protein, 16 aa, Artificial Sequence
  Description of Artificial Sequence Synthetic peptide
QSQQTFXNLWRLLXQN

SEQ ID NO: 136 - protein, 16 aa, Artificial Sequence
  Description of Artificial Sequence Synthetic peptide
QSQQTFXNLWRLLXQN

SEQ ID NO: 137 - protein, 16 aa, Artificial Sequence
  Description of Artificial Sequence Synthetic peptide
QSQQTFXNLWRLLXQN

SEQ ID NO: 138 - protein, 16 aa, Artificial Sequence
  Description of Artificial Sequence Synthetic peptide
QSQQTFXNLWRLLXQN

SEQ ID NO: 139 - protein, 22 aa, Artificial Sequence
  Description of Artificial Sequence Synthetic peptide
XIWIAQELRXIGDXFNAYYARR

SEQ ID NO: 140 - protein, 22 aa, Artificial Sequence
  Description of Artificial Sequence Synthetic peptide
XIWIAQELRXIGDXFNAYYARR

SEQ ID NO: 141 - protein, 16 aa, Artificial Sequence
  Description of Artificial Sequence Synthetic peptide
QSQQTFXNLWRLLXQN

SEQ ID NO: 142 - protein, 16 aa, Artificial Sequence
  Description of Artificial Sequence Synthetic peptide
QSQQTFXNLWRLLXQN

SEQ ID NO: 143 - protein, 21 aa, Artificial Sequence
  Description of Artificial Sequence Synthetic peptide
DIIRNIARHLAXVGDXXDRSI

SEQ ID NO: 144 - protein, 21 aa, Artificial Sequence
  Description of Artificial Sequence Synthetic peptide
DIIRNIARHLAXVXDXXDRSI

SEQ ID NO: 145 - protein, 21 aa, Artificial Sequence
  Description of Artificial Sequence Synthetic peptide
DIIRNIARHLAXVGDXXDRSI

SEQ ID NO: 146 - protein, 21 aa, Artificial Sequence
  Description of Artificial Sequence Synthetic peptide
IWIAQELRXIGDXFNAYYARR

SEQ ID NO: 147 - protein, 21 aa, Artificial Sequence
  Description of Artificial Sequence Synthetic peptide
IWIAQELRXIGDXFNAYYARR

SEQ ID NO: 148 - protein, 21 aa, Artificial Sequence
  Description of Artificial Sequence Synthetic peptide
IWIAQELRXIGDXFNAYYARR

SEQ ID NO: 149 - protein, 21 aa, Artificial Sequence
  Description of Artificial Sequence Synthetic peptide
DIIRNIARHLAXVGDXXDRSI

SEQ ID NO: 150 - protein, 21 aa, Artificial Sequence
  Description of Artificial Sequence Synthetic peptide
DIIRNIARHLAXVGDXXDRSI

SEQ ID NO: 151 - protein, 21 aa, Artificial Sequence
  Description of Artificial Sequence Synthetic peptide
IWIAQELRXIGDXFNAYYARR

SEQ ID NO: 152 - protein, 21 aa, Artificial Sequence
  Description of Artificial Sequence Synthetic peptide
DIIRNIARHLAXVGDXXDRSI

(19) United States
(12) Patent Application Publication
(10) Pub. No.: US 2016/0108089 A1
(43) Pub. Date: Apr. 21, 2016

(54) BIOLOGICALLY ACTIVE PEPTIDOMIMETIC MACROCYCLES

(71) Applicant: Aileron Therapeutics, Inc., Cambridge, MA (US)

(72) Inventors: Huw W. NASH, Concord, MA (US); Rosana KAPELLER-LIBERMANN, Chestnut Hill, MA (US); Tomi K. SAWYER, Southborough, MA (US); Noriyuki KAWAHATA, Somerville, MA (US); Vincent GUERLAVAIS, Arlington, MA (US); Matthew IADANZA, Boston, MA (US)

(21) Appl. No.: 14/718,288

(22) Filed: May 21, 2015

Related U.S. Application Data
(63) Continuation of application No. 14/156,350, filed on Jan. 15, 2014, now abandoned.
Application No. 14/156,350 is a continuation of application No. 13/570,146, filed on Aug. 8, 2012, now abandoned.
Application No. 13/570,146 is a continuation of application No. 12/420,816, filed on Apr. 8, 2009, now abandoned.
(60) Provisional application No. 61/043,346, filed on Apr. 8, 2008.

(51) Int. Cl. C07K 7/56; G01N 33/68

(52) U.S. Cl. CPC C07K 7/56; G01N 33/68; G01N 2500/04

(57) ABSTRACT

The present invention provides biologically active peptidomimetic macrocycles with improved properties relative to their corresponding polypeptides. The invention additionally provides methods of preparing and using such macrocycles, for example in therapeutic applications.

### CROSS-REFERENCE

This application is a Continuation Application which claims the benefit of Ser. No. 14/156,350, filed Jan. 15, 2014, which is a continuation of U.S. application Ser. No. 13/570,146, filed Aug. 8, 2012; which is a continuation of U.S. patent application Ser. No. 12/420,816, filed Apr. 8, 2009, which claims the benefit of U.S. Provisional Application No. 61/043,346, filed Apr. 8, 2008, each of which application is incorporated herein in its entirety by reference.

### BACKGROUND OF THE INVENTION

Peptides are becoming increasingly important in pharmaceutical applications. Unmodified peptides often suffer from poor metabolic stability, poor cell penetrability, and promiscuous binding due to conformational flexibility. To improve these properties, researchers have generated cyclic peptides and peptidomimetics by a variety of methods, including disulfide bond formation, amide bond formation, and carbon-carbon bond formation (Jackson et al. (1991), J. Am. Chem. Soc. 113:9391-9392; Phelan et al. (1997), J. Am. Chem. Soc. 119:455-460; Taylor (2002), Biopolymers 66: 49-75; Brunel et al. (2005), Chem. Commun. (20):2552-2554; Hiroshige et al. (1995), J. Am. Chem. Soc. 117: 11590-11591; Blackwell et al. (1998), Angew. Chem. Int. Ed. 37:3281-3284; Schafmeister et al. (2000), J. Am. Chem. Soc. 122:5891-5892). Limitations of these methods include poor metabolic stability (disulfide and amide bonds), poor cell penetrability (disulfide and amide bonds), and the use of potentially toxic metals (for carbon-carbon bond formation). Thus, there is a significant need for improved methods to produce peptides or peptidomimetics that possess increased biological activity, for example conformational rigidity, metabolic stability and cell penetrability. The present invention addresses these and other needs in the art.

### SUMMARY OF THE INVENTION

The present invention provides biologically active peptidomimetic macrocycles with improved properties relative to a corresponding crosslinked polypeptide.

In one embodiment, the present invention provides a method of improving a biological activity of a polypeptide comprising the step of providing a crosslinked alpha-helical polypeptide comprising a crosslinker wherein a hydrogen atom attached to an α-carbon atom of an amino acid of said crosslinked polypeptide is replaced with a substituent of formula R-, wherein R- is alkyl, alkenyl, alkynyl, arylalkyl, cycloalkylalkyl, heteroalkyl, or heterocycloalkyl, unsubstituted or substituted with halo-; and the biological activity of said polypeptide is improved at least 2-fold relative to a corresponding polypeptide lacking said substituent. In some embodiments, the biological activity of said polypeptide is increased on average at least 2-fold. In other embodiments, the biological activity of said polypeptide is increased at least 5-fold, 10-fold, or 15-fold. In yet other embodiments, the biological activity of said polypeptide is decreased on average at least 2-fold, 5-fold, 10-fold, or 15-fold.

In some embodiments, the crosslinker connects two α-carbon atoms. In other embodiments, two α-carbon atoms are substituted with independent substituents of formula R-. In one embodiment, one α-carbon atom to which the crosslinker is attached is substituted with a substituent of formula R-. In another embodiment, two α-carbon atoms to which the crosslinker is attached are substituted with independent substituents of formula R-. In an alternative embodiment, one α-carbon atom to which the crosslinker is not attached is substituted with a substituent of formula R-. For example, two α-carbon atoms to which the crosslinker is not attached can be substituted with independent substituents of formula R-.

In one embodiment of the methods of the invention, R- is alkyl. For example, R- is methyl. Alternatively, R- and any portion of the crosslinker taken together can form a cyclic structure. In another embodiment, the crosslinker is formed of consecutive carbon-carbon bonds. For example, the crosslinker may comprise at least 8, 9, 10, 11, or 12 consecutive bonds. In other embodiments, the crosslinker may comprise at least 7, 8, 9, 10, or 11 carbon atoms.

In another embodiment, the crosslinked polypeptide comprises an α-helical domain of a BCL-2 family member. For example, the crosslinked polypeptide comprises a BH3 domain. In other embodiments, the crosslinked polypeptide comprises at least 60%, 70%, 80%, 85%, 90% or 95% of any of the sequences in Tables 1, 2, 3 and 4.

In some embodiments, the improved biological activity includes increased cell penetrability, increased α-helicity, improved binding to a target protein, and/or improved binding to any BCL-2 family protein. In other embodiments, the improved biological activity includes increased half-life in the presence of protease, decreased rate of degradation by a protease, and/or increased ability to induce apoptosis.

In still other embodiments, the biological activity is measured as the percentage of the number of cells killed in an in vitro assay in which cultured cells are exposed to an effective concentration of said polypeptide. Alternatively, the improved biological activity includes increased structural stability, increased stability in blood, increased intracellular stability, increased in vivo stability, increased chemical stability, improved physicochemical properties and/or increased formulation properties.

Also provided is a method for preparing a cross-linked polypeptide comprising a) providing a precursor polypeptide comprising at least two moieties capable of undergoing reaction to form a covalent bond between said two moieties, wherein at least one of said moieties is attached to an α-carbon atom of an amino acid of said crosslinked polypeptide, and wherein at least two isomers may be obtained following said reaction; b) replacing a hydrogen atom attached to said α-carbon atom with a substituent of formula R-, wherein R- is alkyl, alkenyl, alkynyl, arylalkyl, cycloalkylalkyl, heteroalkyl, or heterocycloalkyl, unsubstituted or substituted with halo-; and c) incubating said precursor polypeptide in conditions that promote formation of at least one crosslink between said moieties, wherein one of said at least two isomers is obtained in a greater yield than another of said at least two isomers. In some embodiments, the ratio of said at least two isomers obtained is greater than 2:1, 3:1, 5:1 or 10:1. In other embodiments, the crosslinker connects two α-carbon atoms. In still other embodiments, the crosslinked polypeptide comprises an alpha-helix.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 describes the biological activity of several peptidomimetic macrocycles macrocycles (SEQ ID NOS 125, 143-144 and 126, respectively, in order of appearance) of the invention.

FIG. 2 illustrates the increase in biological activity in a peptidomimetic macrocycle in which each α-carbon atom to which the crosslinker is attached is substituted with a methyl group compared to a corresponding macrocycle in which each α-carbon atom to which the crosslinker is attached is substituted with a hydrogen atom.

FIG. 3 illustrates the increase in biological activity in a peptidomimetic macrocycle in which one α-carbon atom to which the crosslinker is not attached is substituted with two methyl groups compared to a corresponding macrocycle in which one α-carbon atom to which the crosslinker is not attached is substituted with two hydrogen atoms.

FIG. 4 depicts binding properties to GST-Mcl-1 of SP-4 and SP-54 peptidomimetic macrocycles.

FIG. 5 depicts binding properties to GST-Bcl-2 of SP-4 and SP-54 peptidomimetic macrocycles.

FIG. 6 depicts receptor binding assay results for SP-27 and SP-28 peptidomimetic macrocycles.

FIG. 7 depicts binding properties to GST-Bcl-XL of SP-1 and SP-35 peptidomimetic macrocycles.

FIG. 8 depicts binding properties to GST-Bcl-2 of SP-1 and SP-35 peptidomimetic macrocycles.

FIGS. 9, 10 and 11 compare penetration of fluorescently-labeled SP-50 and SP-51 p53 peptidomimetic macrocycles into SJSA-1 cells.

FIG. 12 describes the comparative pepsin stability of SP-1 and SP-35 peptidomimetic macrocycles of the invention.

FIG. 13 describes the comparative pepsin stability of SP-36 and SP-37 peptidomimetic macrocycles of the invention.

FIG. 14 describes the comparative pepsin stability of SP-33 and SP-34 peptidomimetic macrocycles of the invention.

FIG. 15 describes the comparative trypsin stability of SP-42 and SP-43 peptidomimetic macrocycles of the invention.

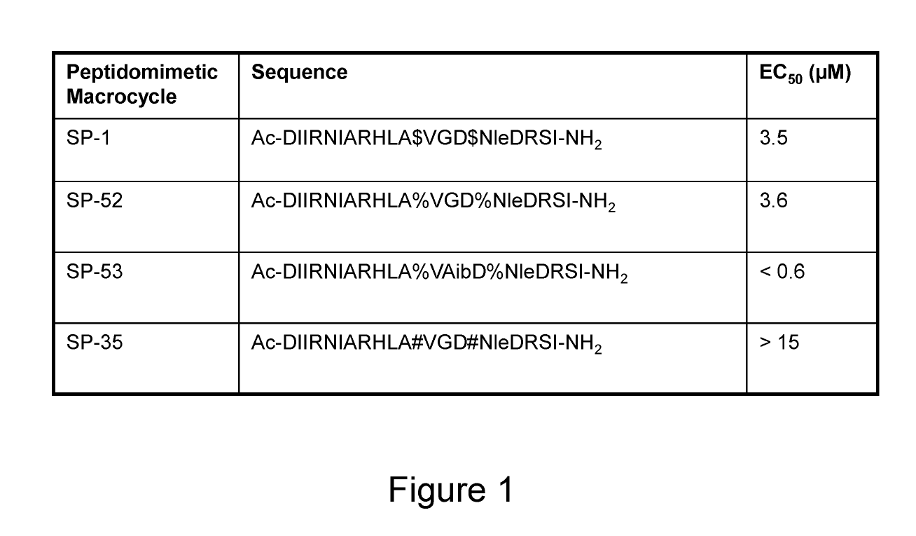

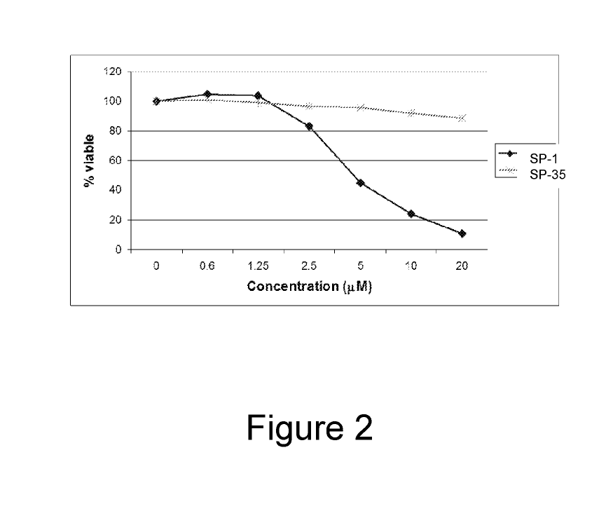

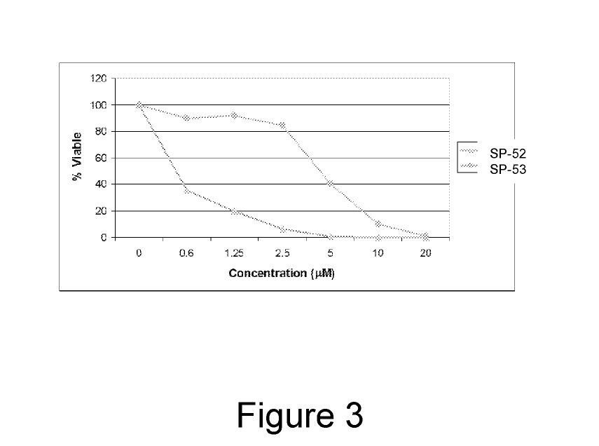

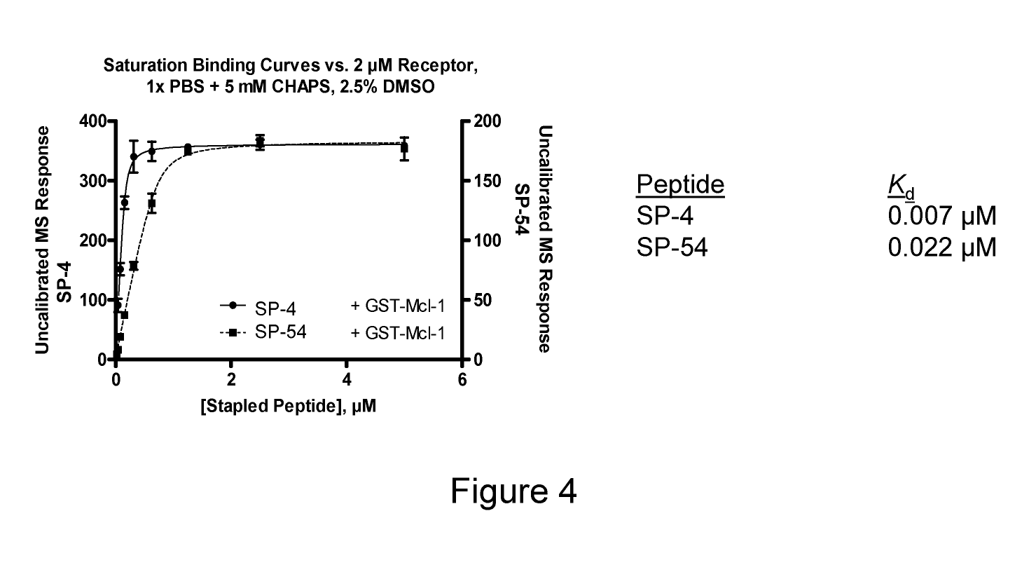

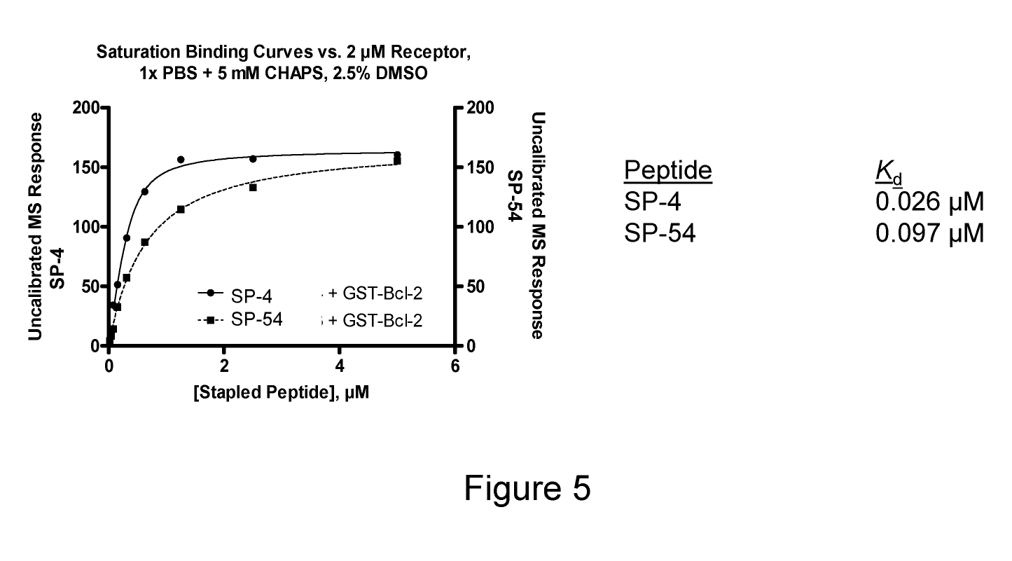

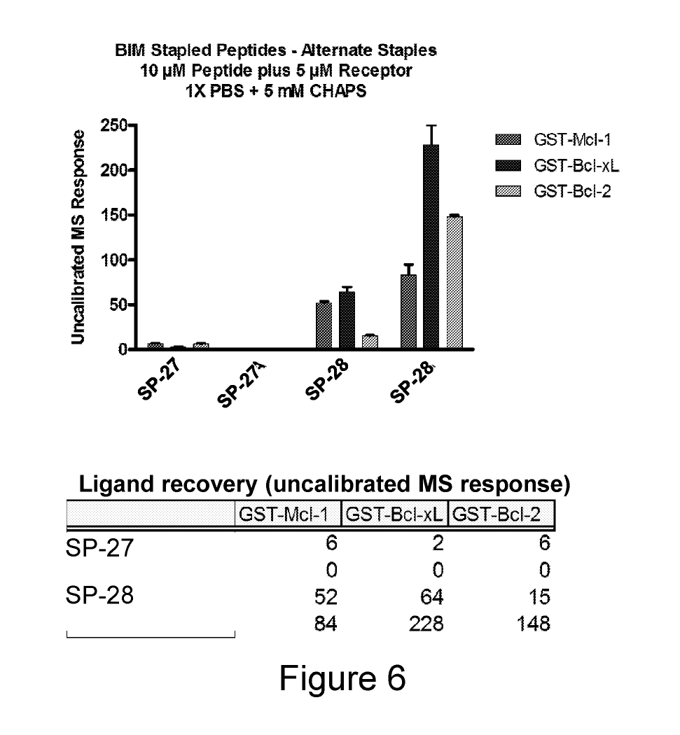

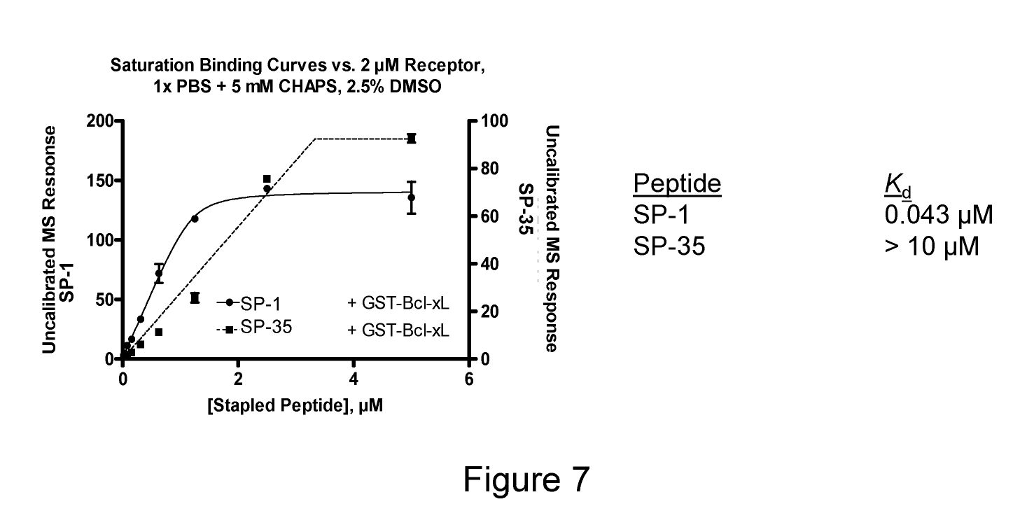

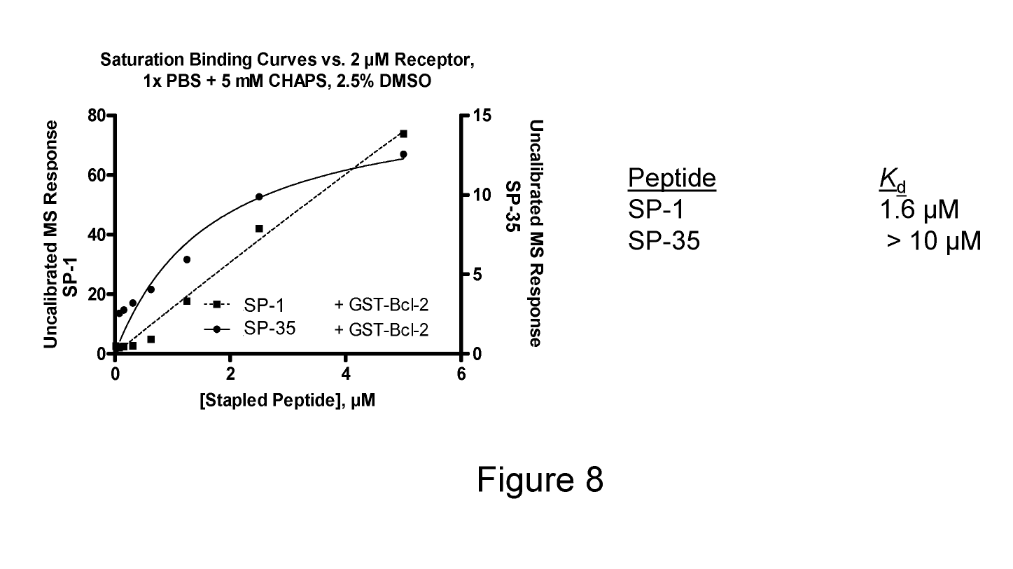

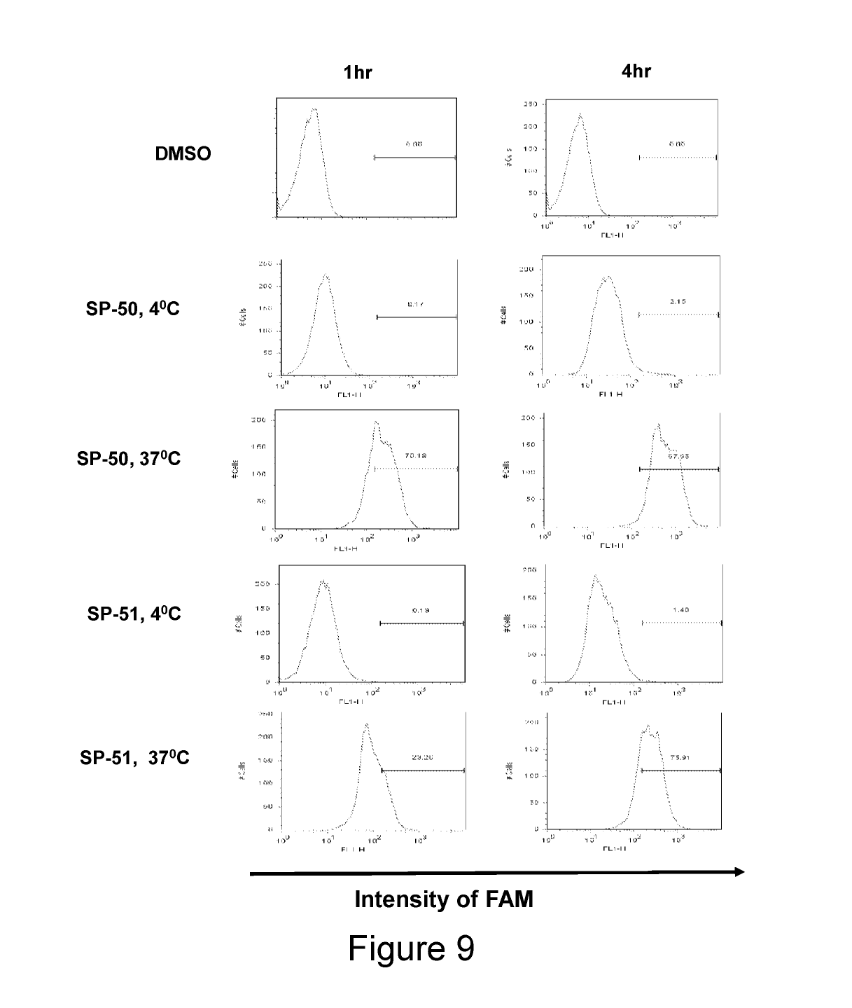

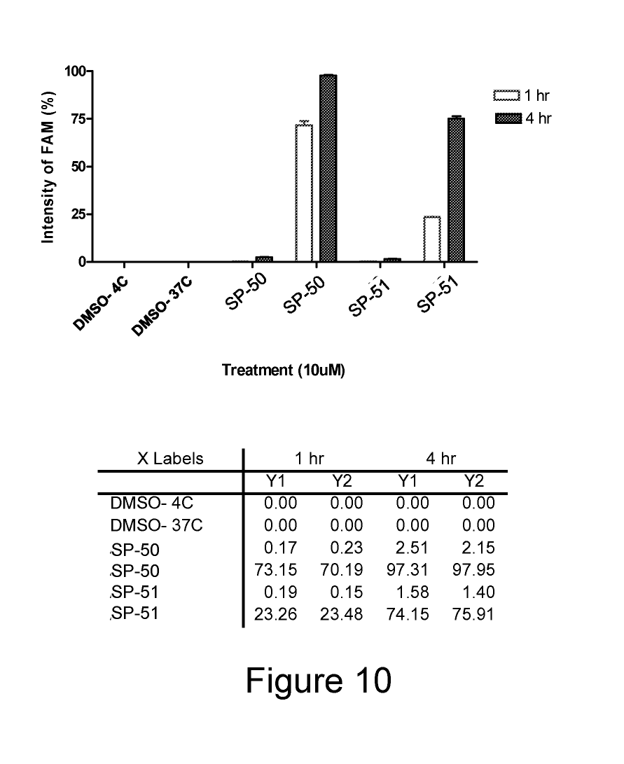

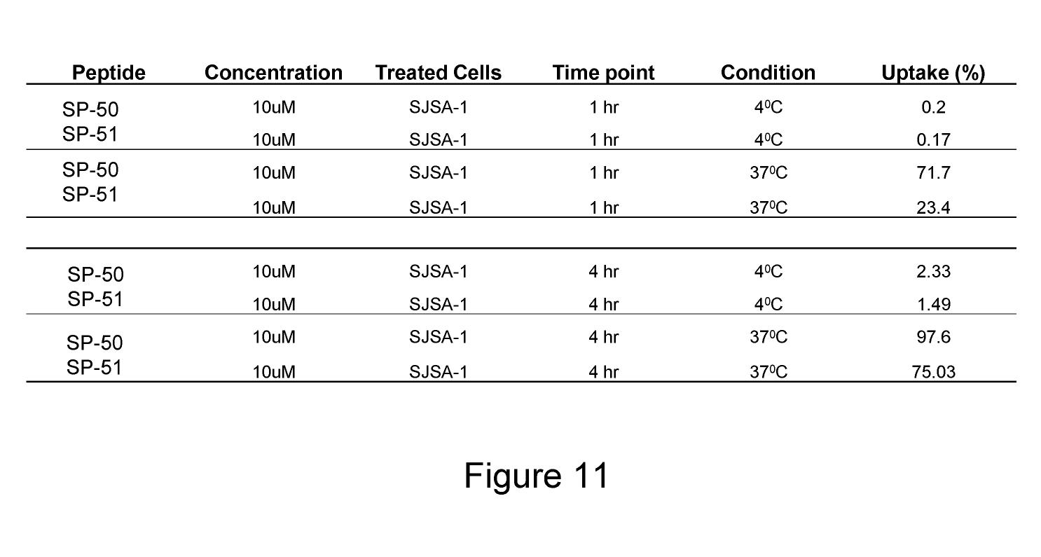

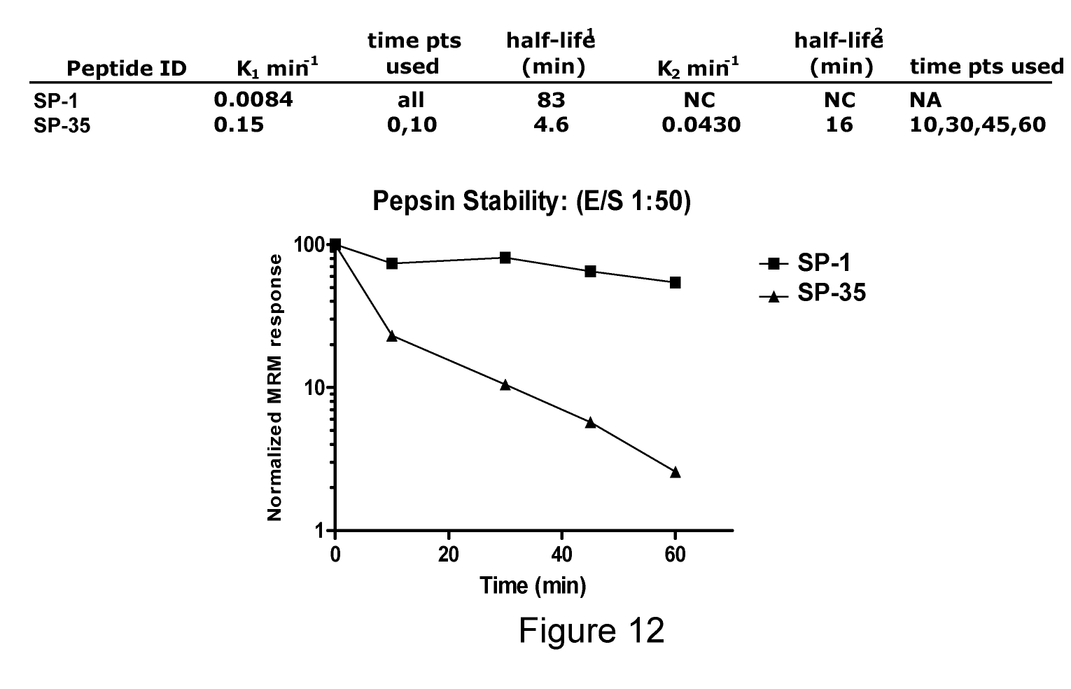

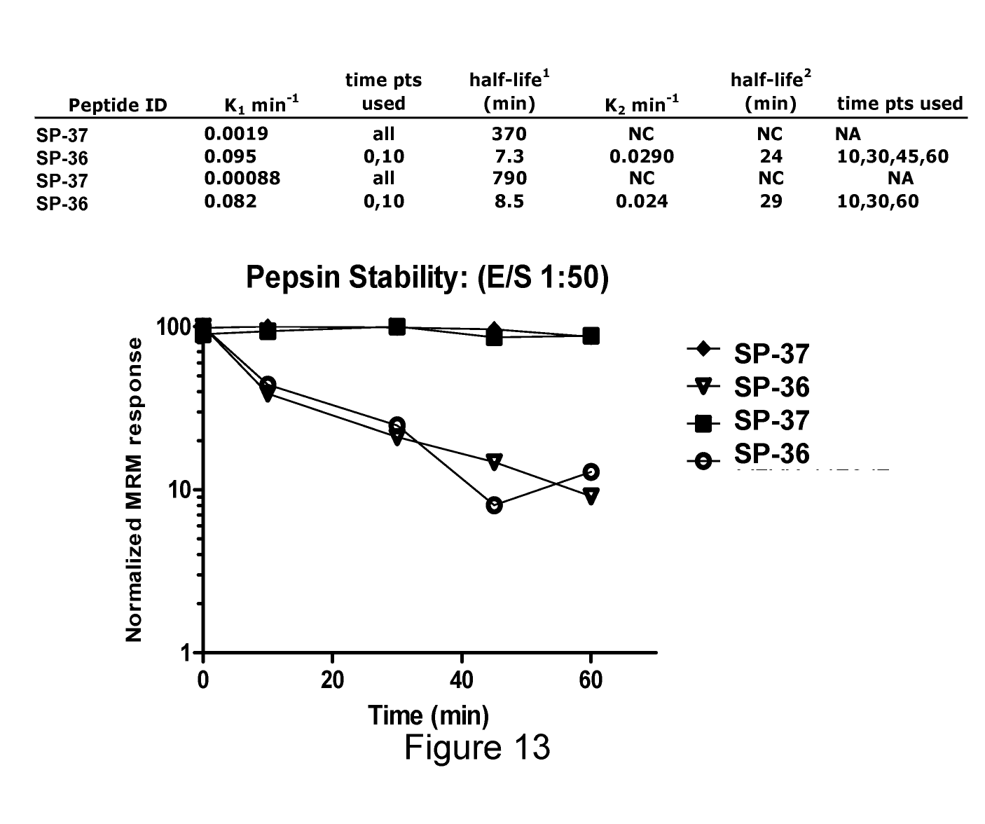

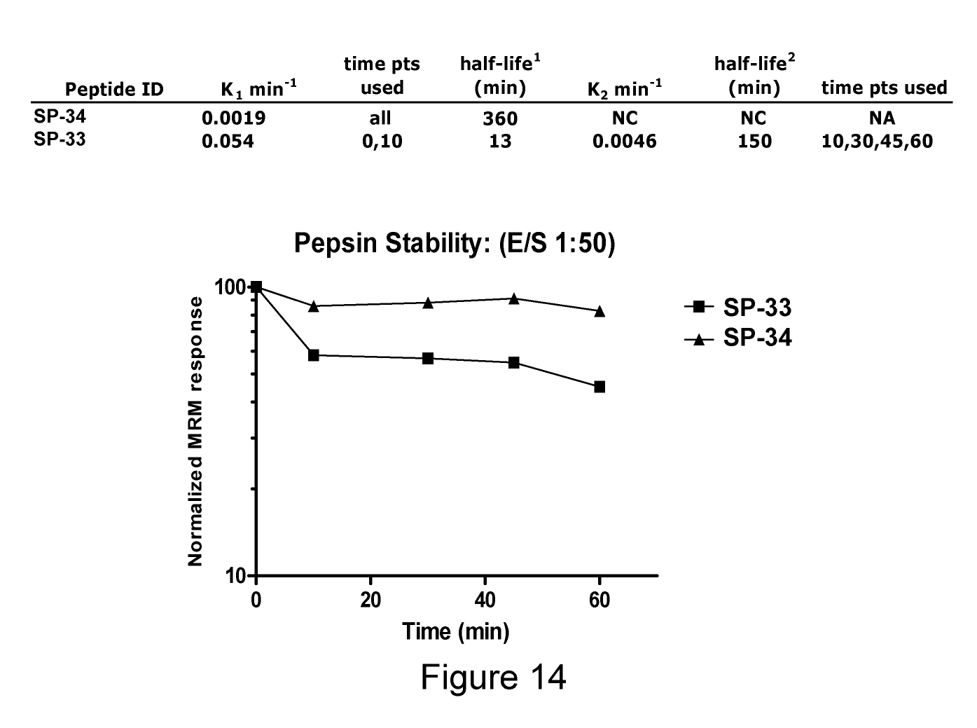

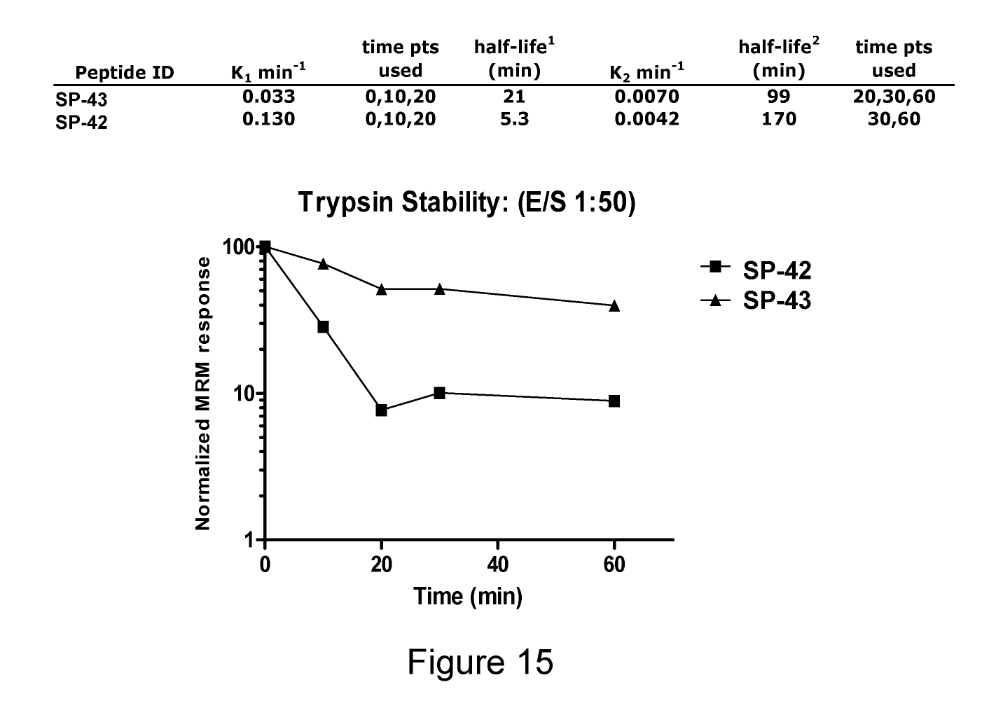

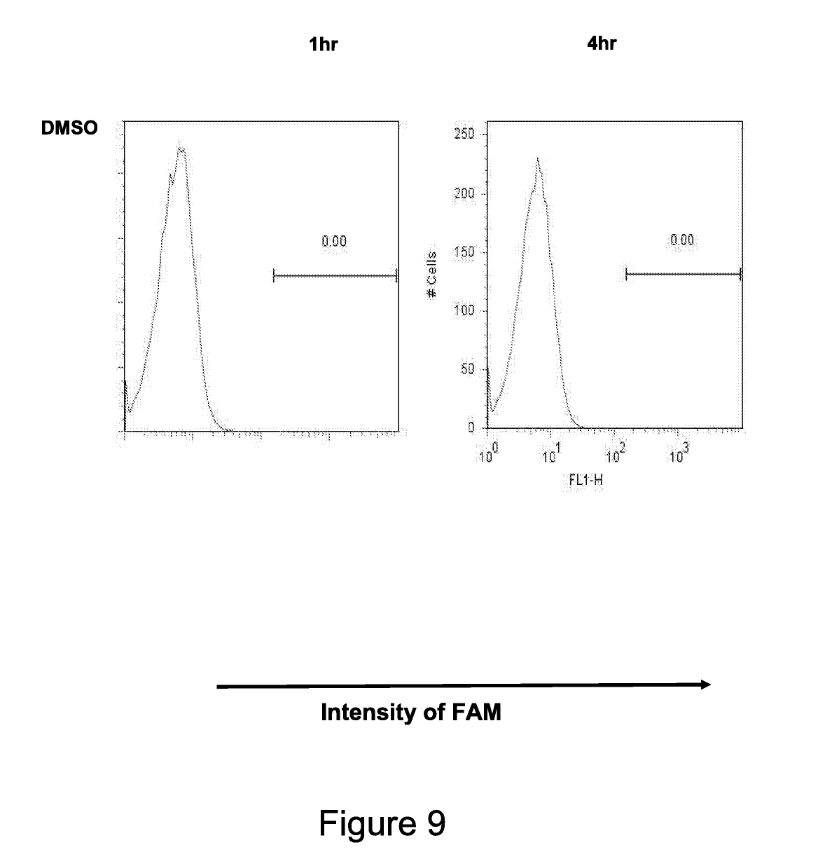

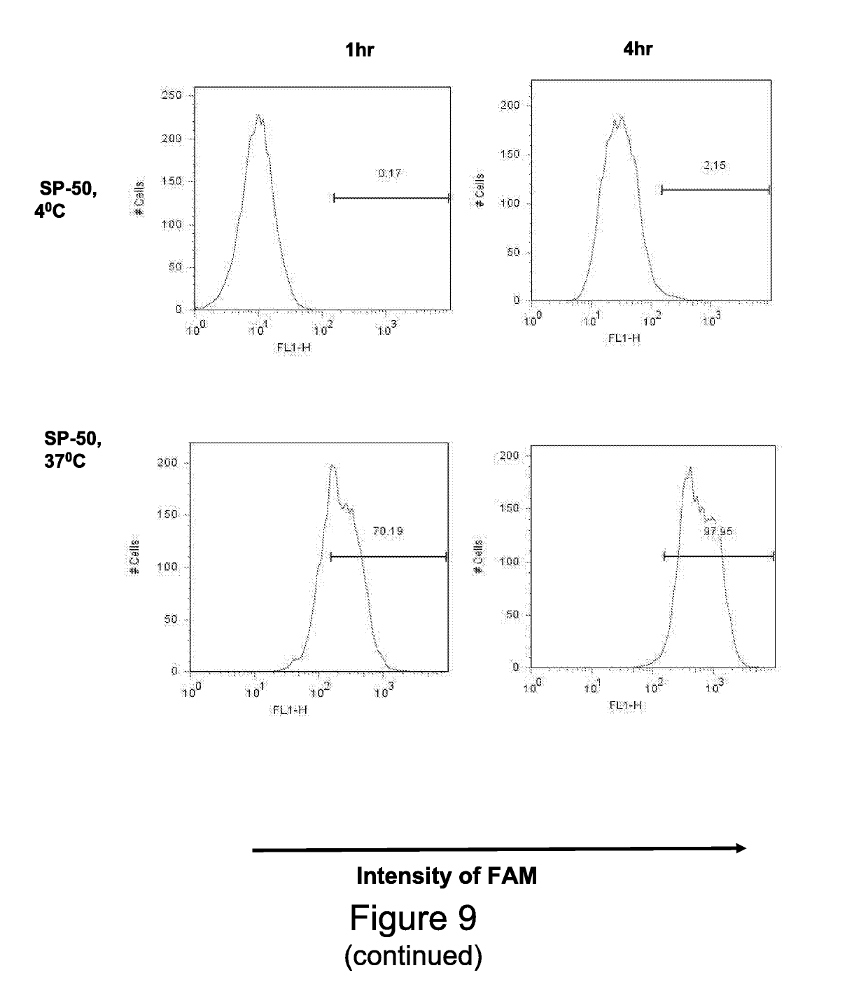

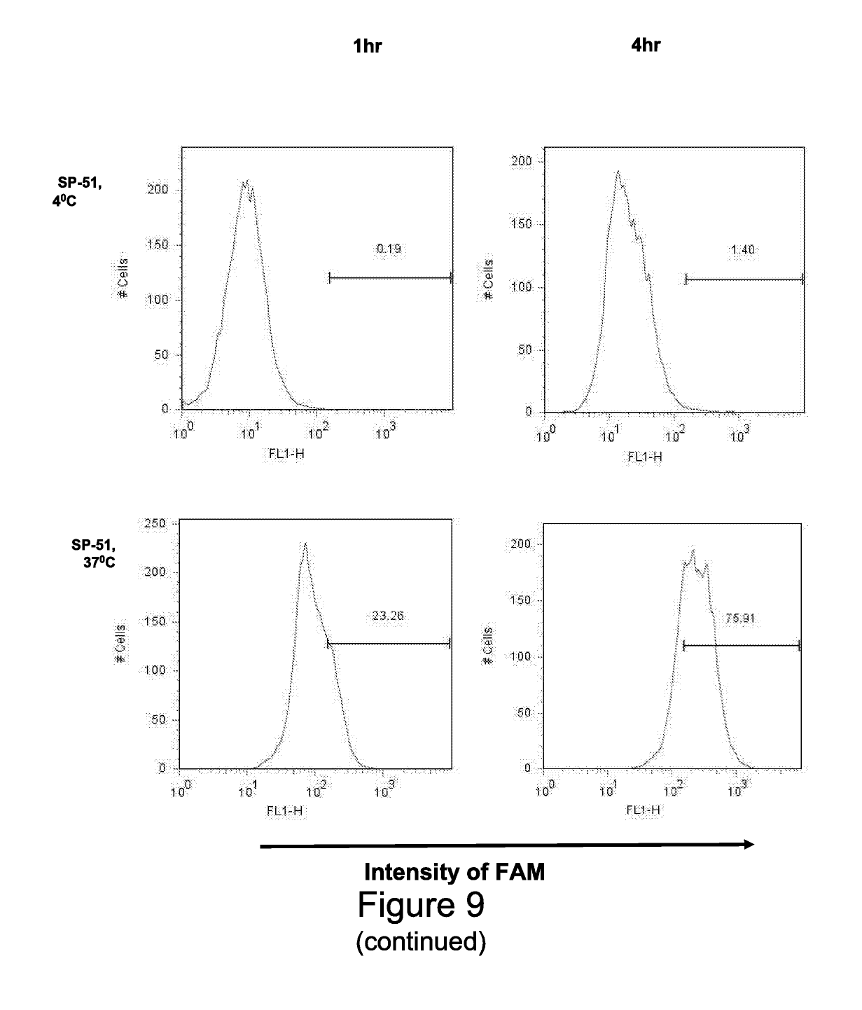

### DETAILED DESCRIPTION OF THE INVENTION

As used herein, the terms “treating” and “to treat”, mean to alleviate symptoms, eliminate the causation either on a temporary or permanent basis, or to prevent or slow the appearance of symptoms. The term “treatment” includes alleviation, elimination of causation (temporary or permanent) of, or prevention of symptoms and disorders associated with any condition. The treatment may be a pre-treatment as well as a treatment at the onset of symptoms.

The term “standard method of care” refers to any therapeutic or diagnostic method, compound, or practice which is part of the standard of care for a particular indication. The “standard of care” may be established by any authority such as a health care provider or a national or regional institute for any diagnostic or treatment process that a clinician should follow for a certain type of patient, illness, or clinical circumstance. Exemplary standard of care methods for various type of cancers are provided for instance by the National Cancer Institute.

As used herein, the term “cell proliferative disorder” encompasses cancer, hyperproliferative disorders, neoplastic disorders, immunoproliferative disorders and other disorders. A “cell proliferative disorder” relates to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. A metastatic tumor can arise from a multitude of primary tumor types, including but not limited to those of breast, lung, liver, colon and ovarian origin. “Pathologic hyperproliferative” cells occur in disease states characterized by malignant tumor growth and immunoproliferative diseases. Examples of non-pathologic hyperproliferative cells include proliferation of cells associated with wound repair. Examples of cellular proliferative and/or differentiative disorders include cancer, e.g., carcinoma, sarcoma, or metastatic disorders.

The term “derived from” in the context of the relationship between a cell line and a related cancer signifies that the cell line may be established from any cancer in a specific broad category of cancers.

As used herein, the term “macrocycle” refers to a molecule having a chemical structure including a ring or cycle formed by at least 9 covalently bonded atoms.

As used herein, the term “peptidomimetic macrocycle”, “crosslinked polypeptide” or “stapled peptide” refers to a compound comprising a plurality of amino acid residues joined by a plurality of peptide bonds and at least one macrocycle-forming linker which forms a macrocycle between a first naturally-occurring or non-naturally-occurring amino acid residue (or analog) and a second naturally-occurring or non-naturally-occurring amino acid residue (or analog) within the same molecule. Peptidomimetic macrocycles include embodiments where the macrocycle-forming linker connects the α carbon of the first amino acid residue (or analog) to the α carbon of the second amino acid residue (or analog). The peptidomimetic macrocycles optionally include one or more non-peptide bonds between one or more amino acid residues and/or amino acid analog residues, and optionally include one or more non-naturally-occurring amino acid residues or amino acid analog residues in addition to any which form the macrocycle.

As used herein, the term “stability” refers to the maintenance of a defined secondary structure in solution by a peptidomimetic macrocycle of the invention as measured by circular dichroism, NMR or another biophysical measure, or resistance to proteolytic degradation in vitro or in vivo. Non-limiting examples of secondary structures contemplated in this invention are α-helices, β-turns, and β-pleated sheets.

As used herein, the term “helical stability” refers to the maintenance of a helical structure by a peptidomimetic macrocycle of the invention as measured by circular dichroism or NMR. For example, in some embodiments, the peptidomimetic macrocycles of the invention exhibit at least a 1.25, 1.5, 1.75 or 2-fold increase in α-helicity as determined by circular dichroism compared to a corresponding macrocycle lacking the R- substituent.

The term “α-amino acid” or simply “amino acid” refers to a molecule containing both an amino group and a carboxyl group bound to a carbon which is designated the α-carbon. Suitable amino acids include, without limitation, both the D- and L-isomers of the naturally-occurring amino acids, as well as non-naturally occurring amino acids prepared by organic synthesis or other metabolic routes. Unless the context specifically indicates otherwise, the term amino acid, as used herein, is intended to include amino acid analogs.

The term “naturally occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

The term “amino acid analog” or “non-natural amino acid” refers to a molecule which is structurally similar to an amino acid and which can be substituted for an amino acid in the formation of a peptidomimetic macrocycle. Amino acid analogs include, without limitation, compounds which are structurally identical to an amino acid, as defined herein, except for the inclusion of one or more additional methylene groups between the amino and carboxyl group (e.g., α-amino β-carboxy acids), or for the substitution of the amino or carboxy group by a similarly reactive group (e.g., substitution of the primary amine with a secondary or tertiary amine, or substitution or the carboxy group with an ester).

A “non-essential” amino acid residue is a residue that can be altered from the wild-type sequence of a polypeptide (e.g., a BH3 domain or the p53 MDM2 binding domain) without abolishing or substantially altering its essential biological or biochemical activity (e.g., receptor binding or activation). An “essential” amino acid residue is a residue that, when altered from the wild-type sequence of the polypeptide, results in abolishing or substantially abolishing the polypeptide's essential biological or biochemical activity.

A “conservative amino acid substitution” is one in which the amino acid residue is replaced with an amino acid residue having a similar side chain. Families of amino acid residues having similar side chains have been defined in the art. These families include amino acids with basic side chains (e.g., K, R, H), acidic side chains (e.g., D, E), uncharged polar side chains (e.g., G, N, Q, S, T, Y, C), nonpolar side chains (e.g., A, V, L, I, P, F, M, W), beta-branched side chains (e.g., T, V, I) and aromatic side chains (e.g., Y, F, W, H). Thus, a predicted nonessential amino acid residue in a BH3 polypeptide, for example, is preferably replaced with another amino acid residue from the same side chain family. Other examples of acceptable substitutions are substitutions based on isosteric considerations (e.g. norleucine for methionine) or other properties (e.g. 2-thienylalanine for phenylalanine).

The term “member” as used herein in conjunction with macrocycles or macrocycle-forming linkers refers to the atoms that form or can form the macrocycle, and excludes substituent or side chain atoms. By analogy, cyclodecane, 1,2-difluoro-decane and 1,3-dimethyl cyclodecane are all considered ten-membered macrocycles as the hydrogen or fluoro substituents or methyl side chains do not participate in forming the macrocycle.

The symbol “ [symbol] ” when used as part of a molecular structure refers to a single bond or a trans or cis double bond.

The term “amino acid side chain” refers to a moiety attached to the α-carbon in an amino acid. For example, the amino acid side chain for alanine is methyl, the amino acid side chain for phenylalanine is phenylmethyl, the amino acid side chain for cysteine is thiomethyl, the amino acid side chain for aspartate is carboxymethyl, the amino acid side chain for tyrosine is 4-hydroxyphenylmethyl, etc. Other non-naturally occurring amino acid side chains are also included, for example, those that occur in nature (e.g., an amino acid metabolite) or those that are made synthetically (e.g., an α,□α di-substituted amino acid).

The term “α,□α di-substituted amino” acid refers to a molecule or moiety containing both an amino group and a carboxyl group bound to a carbon (the α-carbon) that is attached to two natural or non-natural amino acid side chains.

The term “polypeptide” encompasses two or more naturally or non-naturally-occurring amino acids joined by a covalent bond (e.g., an amide bond). Polypeptides as described herein include full length proteins (e.g., fully processed proteins) as well as shorter amino acid sequences (e.g., fragments of naturally-occurring proteins or synthetic polypeptide fragments).

The term “macrocyclization reagent” or “macrocycle-forming reagent” as used herein refers to any reagent which may be used to prepare a peptidomimetic macrocycle of the invention by mediating the reaction between two reactive groups. Reactive groups may be, for example, an azide and alkyne, in which case macrocyclization reagents include, without limitation, Cu reagents such as reagents which provide a reactive Cu(I) species, such as CuBr, CuI or CuOTf, as well as Cu(II) salts such as Cu(CO₂CH₃)₂, CuSO₄, and CuCl₂ that can be converted in situ to an active Cu(I) reagent by the addition of a reducing agent such as ascorbic acid or sodium ascorbate. Macrocyclization reagents may additionally include, for example, Ru reagents known in the art such as Cp*RuCl(PPh₃)₂, [Cp*RuCl]₄ or other Ru reagents which may provide a reactive Ru(II) species. In other cases, the reactive groups are terminal olefins. In such embodiments, the macrocyclization reagents or macrocycle-forming reagents are metathesis catalysts including, but not limited to, stabilized, late transition metal carbene complex catalysts such as Group VIII transition metal carbene catalysts. For example, such catalysts are Ru and Os metal centers having a +2 oxidation state, an electron count of 16 and pentacoordinated. Additional catalysts are disclosed in Grubbs et al., “Ring Closing Metathesis and Related Processes in Organic Synthesis” Acc. Chem. Res. 1995, 28, 446-452, and U.S. Pat. No. 5,811,515. In yet other cases, the reactive groups are thiol groups. In such embodiments, the macrocyclization reagent is, for example, a linker functionalized with two thiol-reactive groups such as halogen groups.

The term “halo” or “halogen” refers to fluorine, chlorine, bromine or iodine or a radical thereof.

The term “alkyl” refers to a hydrocarbon chain that is a straight chain or branched chain, containing the indicated number of carbon atoms. For example, C₁-C₁₀ indicates that the group has from 1 to 10 (inclusive) carbon atoms in it. In the absence of any numerical designation, “alkyl” is a chain (straight or branched) having 1 to 20 (inclusive) carbon atoms in it.

The term “alkylene” refers to a divalent alkyl (i.e., -R-).

The term “alkenyl” refers to a hydrocarbon chain that is a straight chain or branched chain having one or more carbon-carbon double bonds. The alkenyl moiety contains the indicated number of carbon atoms. For example, C₂-C₁₀ indicates that the group has from 2 to 10 (inclusive) carbon atoms in it. The term “lower alkenyl” refers to a C₂-C₆ alkenyl chain. In the absence of any numerical designation, “alkenyl” is a chain (straight or branched) having 2 to 20 (inclusive) carbon atoms in it.

The term “alkynyl” refers to a hydrocarbon chain that is a straight chain or branched chain having one or more carbon-carbon triple bonds. The alkynyl moiety contains the indicated number of carbon atoms. For example, C₂-C₁₀ indicates that the group has from 2 to 10 (inclusive) carbon atoms in it. The term “lower alkynyl” refers to a C₂-C₆ alkynyl chain. In the absence of any numerical designation, “alkynyl” is a chain (straight or branched) having 2 to 20 (inclusive) carbon atoms in it.

The term “aryl” refers to a 6-carbon monocyclic or 10-carbon bicyclic aromatic ring system wherein 0, 1, 2, 3, or 4 atoms of each ring are substituted by a substituent. Examples of aryl groups include phenyl, naphthyl and the like. The term “arylalkyl” or the term “aralkyl” refers to alkyl substituted with an aryl. The term “arylalkoxy” refers to an alkoxy substituted with aryl.

“Arylalkyl” refers to an aryl group, as defined above, wherein one of the aryl group's hydrogen atoms has been replaced with a C₁-C₅ alkyl group, as defined above. Representative examples of an arylalkyl group include, but are not limited to, 2-methylphenyl, 3-methylphenyl, 4-methylphenyl, 2-ethylphenyl, 3-ethylphenyl, 4-ethylphenyl, 2-propylphenyl, 3-propylphenyl, 4-propylphenyl, 2-butylphenyl, 3-butylphenyl, 4-butylphenyl, 2-pentylphenyl, 3-pentylphenyl, 4-pentylphenyl, 2-isopropylphenyl, 3-isopropylphenyl, 4-isopropylphenyl, 2-isobutylphenyl, 3-isobutylphenyl, 4-isobutylphenyl, 2-sec-butylphenyl, 3-sec-butylphenyl, 4-sec-butylphenyl, 2-t-butylphenyl, 3-t-butylphenyl and 4-t-butylphenyl.

“Arylamido” refers to an aryl group, as defined above, wherein one of the aryl group's hydrogen atoms has been replaced with one or more —C(O)NH₂ groups. Representative examples of an arylamido group include 2-C(O)NH2-phenyl, 3-C(O)NH₂-phenyl, 4-C(O)NH₂-phenyl, 2-C(O)NH₂-pyridyl, 3-C(O)NH₂-pyridyl, and 4-C(O)NH₂-pyridyl,

“Alkylheterocycle” refers to a C₁-C₅ alkyl group, as defined above, wherein one of the C₁-C₅ alkyl group's hydrogen atoms has been replaced with a heterocycle. Representative examples of an alkylheterocycle group include, but are not limited to, —CH₂CH₂-morpholine, —CH₂CH₂-piperidine, —CH₂CH₂CH₂-morpholine, and —CH₂CH₂CH₂-imidazole.

“Alkylamido” refers to a C₁-C₅ alkyl group, as defined above, wherein one of the C₁-C₅ alkyl group's hydrogen atoms has been replaced with a —C(O)NH₂ group. Representative examples of an alkylamido group include, but are not limited to, —CH₂—C(O)NH₂, —CH₂CH₂—C(O)NH₂, —CH₂CH₂CH₂C(O)NH₂, —CH₂CH₂CH₂CH₂C(O)NH₂, —CH₂CH₂CH₂CH₂CH₂C(O)NH₂, —CH₂CH(C(O)NH₂)CH₃, —CH₂CH(C(O)NH₂)CH₂CH₃, —CH(C(O)NH₂)CH₂CH₃, —C(CH₃)₂CH₂C(O)NH₂, —CH₂—CH₂—NH—C(O)—CH₃, —CH₂—CH₂—NH—C(O)—CH₃—CH3, and —CH₂—CH₂—NH—C(O)—CH═CH₂.

“Alkanol” refers to a C₁-C₅ alkyl group, as defined above, wherein one of the C₁-C₅ alkyl group's hydrogen atoms has been replaced with a hydroxyl group. Representative examples of an alkanol group include, but are not limited to, —CH₂OH, —CH₂CH₂OH, —CH₂CH₂CH₂OH, —CH₂CH₂CH₂CH₂OH, —CH₂CH₂CH₂CH₂CH₂OH, —CH₂CH(OH)CH₃, —CH₂CH(OH)CH₂CH₃, —CH(OH)CH₃ and —C(CH₃)₂CH₂OH.

“Alkylcarboxy” refers to a C₁-C₅ alkyl group, as defined above, wherein one of the C₁-C₅ alkyl group's hydrogen atoms has been replaced with a —COOH group. Representative examples of an alkylcarboxy group include, but are not limited to, —CH₂COOH, —CH₂CH₂COOH, —CH₂CH₂CH₂COOH, —CH₂CH₂CH₂CH₂COOH, —CH₂CH(COOH)CH₃, —CH₂CH₂CH₂CH₂CH₂COOH, —CH₂CH(COOH)CH₂CH₃, —CH(COOH)CH₂CH₃ and —C(CH₃)₂CH₂COOH.

The term “cycloalkyl” as employed herein includes saturated and partially unsaturated cyclic hydrocarbon groups having 3 to 12 carbons, preferably 3 to 8 carbons, and more preferably 3 to 6 carbons, wherein the cycloalkyl group additionally is optionally substituted. Some cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of O, N, or S if monocyclic, bicyclic, or tricyclic, respectively), wherein 0, 1, 2, 3, or 4 atoms of each ring are substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like.

The term “heteroarylalkyl” or the term “heteroaralkyl” refers to an alkyl substituted with a heteroaryl. The term “heteroarylalkoxy” refers to an alkoxy substituted with heteroaryl.

The term “heteroarylalkyl” or the term “heteroaralkyl” refers to an alkyl substituted with a heteroaryl. The term “heteroarylalkoxy” refers to an alkoxy substituted with heteroaryl.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of O, N, or S if monocyclic, bicyclic, or tricyclic, respectively), wherein 0, 1, 2 or 3 atoms of each ring are substituted by a substituent. Examples of heterocyclyl groups include piperazinyl, pyrrolidinyl, dioxanyl, morpholinyl, tetrahydrofuranyl, and the like.

The term “substituent” refers to a group replacing a second atom or group such as a hydrogen atom on any molecule, compound or moiety. Suitable substituents include, without limitation, halo, hydroxy, mercapto, oxo, nitro, haloalkyl, alkyl, alkaryl, aryl, aralkyl, alkoxy, thioalkoxy, aryloxy, amino, alkoxycarbonyl, amido, carboxy, alkanesulfonyl, alkylcarbonyl, and cyano groups.

In some embodiments, the compounds of this invention contain one or more asymmetric centers and thus occur as racemates and racemic mixtures, single enantiomers, individual diastereomers and diastereomeric mixtures. All such isomeric forms of these compounds are included in the present invention unless expressly provided otherwise. In some embodiments, the compounds of this invention are also represented in multiple tautomeric forms, in such instances, the invention includes all tautomeric forms of the compounds described herein (e.g., if alkylation of a ring system results in alkylation at multiple sites, the invention includes all such reaction products). All such isomeric forms of such compounds are included in the present invention unless expressly provided otherwise. All crystal forms of the compounds described herein are included in the present invention unless expressly provided otherwise.

As used herein, the terms “increase” and “decrease” mean, respectively, to cause a statistically significantly (i.e., p<0.1) increase or decrease of at least 5%.

As used herein, the recitation of a numerical range for a variable is intended to convey that the invention may be practiced with the variable equal to any of the values within that range. Thus, for a variable which is inherently discrete, the variable is equal to any integer value within the numerical range, including the end-points of the range. Similarly, for a variable which is inherently continuous, the variable is equal to any real value within the numerical range, including the end-points of the range. As an example, and without limitation, a variable which is described as having values between 0 and 2 takes the values 0, 1 or 2 if the variable is inherently discrete, and takes the values 0.0, 0.1, 0.01, 0.001, or any other real values ≧0 and ≦2 if the variable is inherently continuous.

As used herein, unless specifically indicated otherwise, the word “or” is used in the inclusive sense of “and/or” and not the exclusive sense of “either/or.”

The term “on average” represents the mean value derived from performing at least three independent replicates for each data point.

The term “biological activity” encompasses structural and functional properties of a macrocycle of the invention. Biological activity is, for example, structural stability, alpha-helicity, affinity for a target, resistance to proteolytic degradation, cell penetrability, intracellular stability, in vivo stability, or any combination thereof.

The details of one or more particular embodiments of the invention are set forth in the accompanying drawings and the description below. Other features, objects, and advantages of the invention will be apparent from the description and drawings, and from the claims.

### Biological Properties of the Peptidomimetic Macrocycles of the Invention

In one aspect, the invention provides a method of improving a biological activity of a peptidomimetic macrocycle. For example, the method is performed by providing a crosslinked alpha-helical polypeptide comprising a crosslinker wherein a hydrogen atom attached to an α-carbon atom of an amino acid of said crosslinked polypeptide is replaced with a substituent of formula R-, wherein the biological activity of said polypeptide is improved relative to a corresponding polypeptide lacking the substituent.

In one embodiment, the increased biological activity includes increased structural stability, increased affinity for a target, increased resistance to proteolytic degradation, decreased rate of degradation by a protease, increased stability in blood, increased intracellular stability, increased in vivo stability, increased in vivo exposure levels, and/or increased cell penetrability when compared to a corresponding macrocycle lacking the R- substituent. In another embodiment, a peptidomimetic macrocycle comprises one or more α-helices in aqueous solutions and/or exhibits an increased degree of α-helicity in comparison to a corresponding polypeptide of the invention in which R- is hydrogen. In other embodiments, the improved biological activity includes increased binding to any BCL-2 family protein. In still other embodiments, the improved biological activity includes increased ability to induce apoptosis. In yet other embodiments, the biological activity is measured as the percentage of the number of cells killed in an in vitro assay in which cultured cells are exposed to an effective concentration of said polypeptide. In a particular embodiment, the improved biological activity includes increased chemical stability, for example chemical stability of a pharmaceutical formulation of the peptidomimetic macrocycle of the invention. In yet another embodiment, the improved biological activity includes improved physicochemical properties or formulation properties.

For example, the biological activity is improved 2, 5, 10, 15, 20, or more than 25-fold. Alternatively, the biological activity is improved on average 2, 5, 10, 15, 20, or more than 25-fold. Various methods for determining the biological activity of the peptidomimetic macrocycles of the invention are described below.

### Design of the Peptidomimetic Macrocycles of the Invention

Any protein or polypeptide with a known primary amino acid sequence which contains a secondary structure believed to impart biological activity is the subject of the present invention. For example, the sequence of the polypeptide can be analyzed and amino acid analogs containing groups reactive with macrocyclization reagents can be substituted at the appropriate positions. The appropriate positions are determined by ascertaining which molecular surface(s) of the secondary structure is (are) required for biological activity and, therefore, across which other surface(s) the macrocycle forming linkers of the invention can form a macrocycle without sterically blocking the surface(s) required for biological activity. Such determinations are made using methods such as X-ray crystallography of complexes between the secondary structure and a natural binding partner to visualize residues (and surfaces) critical for activity; by sequential mutagenesis of residues in the secondary structure to functionally identify residues (and surfaces) critical for activity; or by other methods. By such determinations, the appropriate amino acids are substituted with the amino acids analogs and macrocycle-forming linkers of the invention. For example, for an α-helical secondary structure, one surface of the helix (e.g., a molecular surface extending longitudinally along the axis of the helix and radially 45-135° about the axis of the helix) may be required to make contact with another biomolecule in vivo or in vitro for biological activity. In such a case, a macrocycle-forming linker is designed to link two α-carbons of the helix while extending longitudinally along the surface of the helix in the portion of that surface not directly required for activity.

In some embodiments of the invention, the peptide sequence is derived from the BCL-2 family of proteins. The BCL-2 family is defined by the presence of up to four conserved BCL-2 homology (BH) domains designated BH1, BH2, BH3, and BH4, all of which include α-helical segments (Chittenden et al. (1995), EMBO 14:5589; Wang et al. (1996), Genes Dev. 10:2859). Anti-apoptotic proteins, such as BCL-2 and BCL-X_{L}, display sequence conservation in all BH domains. Pro-apoptotic proteins are divided into “multidomain” family members (e.g., BAK, BAX), which possess homology in the BH1, BH2, and BH3 domains, and “BH3-domain only” family members (e.g., BID, BAD, BIM, BIK, NOXA, PUMA), that contain sequence homology exclusively in the BH3 amphipathic α-helical segment. BCL-2 family members have the capacity to form homo- and heterodimers, suggesting that competitive binding and the ratio between pro- and anti-apoptotic protein levels dictates susceptibility to death stimuli. Anti-apoptotic proteins function to protect cells from pro-apoptotic excess, i.e., excessive programmed cell death. Additional “security” measures include regulating transcription of pro-apoptotic proteins and maintaining them as inactive conformers, requiring either proteolytic activation, dephosphorylation, or ligand-induced conformational change to activate pro-death functions. In certain cell types, death signals received at the plasma membrane trigger apoptosis via a mitochondrial pathway. The mitochondria can serve as a gatekeeper of cell death by sequestering cytochrome c, a critical component of a cytosolic complex which activates caspase 9, leading to fatal downstream proteolytic events. Multidomain proteins such as BCL-2/BCL-X_{L} and BAK/BAX play dueling roles of guardian and executioner at the mitochondrial membrane, with their activities further regulated by upstream BH3-only members of the BCL-2 family. For example, BID is a member of the BH3-domain only family of pro-apoptotic proteins, and transmits death signals received at the plasma membrane to effector pro-apoptotic proteins at the mitochondrial membrane. BID has the capability of interacting with both pro- and anti-apoptotic proteins, and upon activation by caspase 8, triggers cytochrome c release and mitochondrial apoptosis. Deletion and mutagenesis studies determined that the amphipathic α-helical BH3 segment of pro-apoptotic family members may function as a death domain and thus may represent a critical structural motif for interacting with multidomain apoptotic proteins. Structural studies have shown that the BH3 helix can interact with anti-apoptotic proteins by inserting into a hydrophobic groove formed by the interface of BH1, 2 and 3 domains. Activated BID can be bound and sequestered by anti-apoptotic proteins (e.g., BCL-2 and BCL-X_{L}) and can trigger activation of the pro-apoptotic proteins BAX and BAK, leading to cytochrome c release and a mitochondrial apoptosis program. BAD is also a BH3-domain only pro-apoptotic family member whose expression triggers the activation of BAX/BAK. In contrast to BID, however, BAD displays preferential binding to anti-apoptotic family members, BCL-2 and BCL-X_{L}. Whereas the BAD BH3 domain exhibits high affinity binding to BCL-2, BAD BH3 peptide is unable to activate cytochrome c release from mitochondria in vitro, suggesting that BAD is not a direct activator of BAX/BAK. Mitochondria that over-express BCL-2 are resistant to BID-induced cytochrome c release, but co-treatment with BAD can restore BID sensitivity. Induction of mitochondrial apoptosis by BAD appears to result from either: (1) displacement of BAX/BAK activators, such as BID and BID-like proteins, from the BCL-2/BCL-XL binding pocket, or (2) selective occupation of the BCL-2/BCL-XL binding pocket by BAD to prevent sequestration of BID-like proteins by anti-apoptotic proteins. Thus, two classes of BH3-domain only proteins have emerged, BID-like proteins that directly activate mitochondrial apoptosis, and BAD-like proteins, that have the capacity to sensitize mitochondria to BID-like pro-apoptotics by occupying the binding pockets of multidomain anti-apoptotic proteins. Various α-helical domains of BCL-2 family member proteins amenable to the methodology disclosed herein have been disclosed (Walensky et al. (2004), Science 305:1466; and Walensky et al., U.S. Patent Publication No. 2005/0250680, the entire disclosures of which are incorporated herein by reference).

In other embodiments, the peptide sequence is derived from the tumor suppressor p53 protein which binds to the oncogene protein MDM2. The MDM2 binding site is localized within a region of the p53 tumor suppressor that forms an α helix. In U.S. Pat. No. 7,083,983, the entire contents of which are incorporated herein by reference, Lane et al. disclose that the region of p53 responsible for binding to MDM2 is represented approximately by amino acids 13-31 (PLSQETFSDLWKLLPENNV) (SEQ ID NO: 1) of mature human P53 protein. Other modified sequences disclosed by Lane are also contemplated in the instant invention. Furthermore, the interaction of p53 and MDM2 has been discussed by Shair et al. (1997), Chem. & Biol. 4:791, the entire contents of which are incorporated herein by reference, and mutations in the p53 gene have been identified in virtually half of all reported cancer cases. As stresses are imposed on a cell, p53 is believed to orchestrate a response that leads to either cell-cycle arrest and DNA repair, or programmed cell death. As well as mutations in the p53 gene that alter the function of the p53 protein directly, p53 can be altered by changes in MDM2. The MDM2 protein has been shown to bind to p53 and disrupt transcriptional activation by associating with the transactivation domain of p53. For example, an 11 amino-acid peptide derived from the transactivation domain of p53 forms an amphipathic α-helix of 2.5 turns that inserts into the MDM2 crevice. Thus, in some embodiments, novel α-helix structures generated by the method of the present invention are engineered to generate structures that bind tightly to the helix acceptor and disrupt native protein-protein interactions. These structures are then screened using high throughput techniques to identify optimal small molecule peptides. The novel structures that disrupt the MDM2 interaction are useful for many applications, including, but not limited to, control of soft tissue sarcomas (which over-expresses MDM2 in the presence of wild type p53). These cancers are then, in some embodiments, held in check with small molecules that intercept MDM2, thereby preventing suppression of p53. Additionally, in some embodiments, small molecules disrupters of MDM2-p53 interactions are used as adjuvant therapy to help control and modulate the extent of the p53 dependent apoptosis response in conventional chemotherapy.

A non-limiting exemplary list of suitable peptide sequences for use in the present invention is given below:

TABLE 1

| | | SEQ | Cross-linked Sequence | SEQ |
|---|---|---|---|---|
| Name | Sequence (bold = critical residues) | ID NO: | (X = x-link residue) | ID NO: |
| BH3 peptides | | | | |
| BID-BH3 | QEDIIRNIARHLAQVGDSMDRSIPP | 2 | QEDIIRNIARHLAXVGDXMDRSIPP | 25 |
| BIM-BH3 | DNRPEIWIAQELRRIGDEFNAYYAR | 3 | DNRPEIWIAQELRXIGDXFNAYYAR | 26 |
| BAD-BH3 | NLWAAQRYGRELRRMSDEFVDSFKK | 4 | NLWAAQRYGRELRXMSDXFVDSFKK | 27 |
| PUMA-BH3 | EEQWAREIGAQLRRMADDLNAQYER | 5 | EEQWAREIGAQLRXMADXLNAQYER | 28 |
| Hrk-BH3 | RSSAAQLTAARLKALGDELHQRTM | 6 | RSSAAQLTAARLKXLGDXLHQRTM | 29 |
| NOXAA-BH3 | AELPPEFAAQLRKIGDKVYCTW | 7 | AELPPEFAAQLRXIGDXVYCTW | 30 |
| NOXAB-BH3 | VPADLKDECAQLRRIGDKVNLRQKL | 8 | VPADLKDECAQLRXIGDXVNLRQKL | 31 |
| BMF-BH3 | QHRAEVQIARKLQCIADQFHRLHT | 9 | QHRAEVQIARKLQXIADXFHRLHT | 32 |
| BLK-BH3 | SSAAQLTAARLKALGDELHQRT | 10 | SSAAQLTAARLKXLGDXLHQRT | 33 |
| BIK-BH3 | CMEGSDALALRLACIGDEMDVSLRA | 11 | CMEGSDALALRLAXIGDXMDVSLRA | 34 |
| Bnip3 | DIERRKEVESILKKNSDWIWDWSS | 12 | DIERRKEVESILKXNSDXIWDWSS | 35 |
| BOK-BH3 | GRLAEVCAVLLRLGDELEMIRP | 13 | GRLAEVCAVLLXLGDXLEMIRP | 36 |
| BAX-BH3 | PQDASTKKSECLKRIGDELDSNMEL | 14 | PQDASTKKSECLKXIGD [symbol] LDSNMEL | 37 |
| BAK-BH3 | PSSTMGQVGRQLAIIGDDINRR | 15 | PSSTMGQVGRQLAXIGDXINRR | 38 |
| BCL2L1-BH3 | KQALREAGDEFLLR | 16 | KQALRXAGDXFELR | 39 |
| BCL2-BH3 | LSPPVVHLALALRQAGDDFSRR | 17 | LSPPVVHLALALRXAGDXFSRR | 40 |
| BCL-XL-BH3 | EVIPMAAVKQALREAGDEFLLRY | 18 | EVIPMAAVKQALRXAGDXFELRY | 41 |
| BCL-W-BH3 | PADPLHQAMRAAGDEFLTRF | 19 | PADPLHQAMRXAGDXFETRF | 42 |
| MCL1-BH3 | ATSRKLETLRRVGDGVQRNHETA | 20 | ATSRKLETLRXVGDXVQRNHETA | 43 |
| MTD-BH3 | LAEVCTVLLRLGDELEQIR | 21 | LAEVCTVLLXLGDXLEQIR | 44 |
| MAP-1-BH3 | MTVGELSRALGHENGSLDP | 22 | MTVGELSRALGXENGXLDP | 45 |
| NIX-BH3 | VVEGEKEVEALKKSADWVSDWS | 23 | VVEGEKEVEALKXSADXVSDWS | 46 |
| 4ICD(ERBB4)-BH3 | SMARDPQRYLVIQGDDRMKL | 24 | SMARDPQRYLVXQGDXRMKL | 47 |

Table 1 lists human sequences which target the BH3 binding site and are implicated in cancers, autoimmune disorders, metabolic diseases and other human disease conditions.

TABLE 2

| | | SEQ | Cross-linked Sequence | SEQ |
|---|---|---|---|---|
| Name | Sequence (bold = critical residues) | ID NO: | (X = x-link residue) | ID NO: |
| BH3 peptides | | | | |
| BID-BH3 | QEDIIRNIARHLAQVGDSMDRSIPP | 2 | QEDIIRNIXRHLXQVGDSMDRSIPP | 48 |
| BIM-BH3 | DNRPEIWIAQELRRIGDEFNAYYAR | 3 | DNRPEIWIXQELXRIGDEFNAYYAR | 49 |
| BAD-BH3 | NLWAAQRYGRELRRMSDEFVDSFKK | 4 | NLWAAQRYXRELXRMSDEFVDSFKK | 50 |
| PUMA-BH3 | EEQWAREIGAQLRRMADDLNAQYER | 5 | EEQWAREIXAQLXRMADDLNAQYER | 51 |
| Hrk-BH3 | RSSAAQLTAARLKALGDELHQRTM | 6 | RSSAAQLTXARLXALGDELHQRTM | 52 |
| NOXAA-BH3 | AELPPEFAAQLRKIGDKVYCTW | 7 | AELPPEFXAQLXKIGDKVYCTW | 53 |
| NOXAB-BH3 | VPADLKDECAQLRRIGDKVNLRQKL | 8 | VPADLKDEXAQLXRIGDKVNLRQKL | 54 |
| BMF-BH3 | QHRAEVQIARKLQCIADQFHRLHT | 9 | QHRAEVQIXRKLXCIADQFHRLHT | 55 |
| BLK-BH3 | SSAAQLTAARLKALGDELHQRT | 10 | SSAAQLTXARLXALGDELHQRT | 56 |
| BIK-BH3 | CMEGSDALALRLACIGDEMDVSLRA | 11 | CMEGSDALXLRLXCIGDEMDVSLRA | 57 |
| Bnip3 | DIERRKEVESILKKNSDWIWDWSS | 12 | DIERRKEVXSILXKNSDWIWDWSS | 58 |
| BOK-BH3 | GRLAEVCAVLLRLGDELEMIRP | 13 | GRLAEVXAVLXRLGDELEMIRP | 59 |
| BAX-BH3 | PQDASTKKSECLKRIGDELDSNMEL | 14 | PQDASTKKXECLXRIGDELDSNMEL | 60 |
| BAK-BH3 | PSSTMGQVGRQLAIIGDDINRR | 15 | PSSTMGQVXRQLXIIGDDINRR | 61 |
| BCL2L1-BH3 | KQALREAGDEFELR | 16 | XQALXEAGDEFELR | 62 |
| BCL2-BH3 | LSPPVVHLALALRQAGDDFSRR | 17 | LSPPVVHLXLALXQAGDDFSRR | 63 |
| BCL-XL-BH3 | EVIPMAAVKQALREAGDEFELRY | 18 | EVIPMAAVXQALXEAGDEFELRY | 64 |
| BCL-W-BH3 | PADPLHQAMRAAGDEFETRF | 19 | PADPLXQAMXAAGDEFETRF | 65 |
| MCL1-BH3 | ATSRKLETLRRVGDGVQRNHETA | 20 | ATSRKXETLXRVGDGVQRNHETA | 66 |
| MTD-BH3 | LAEVCTVLLRLGDELEQIR | 21 | LAEVXTVLXRLGDELEQIR | 67 |
| MAP-1-BH3 | MTVGELSRALGHENGSLDP | 22 | MTVGELXRALXHENGSLDP | 68 |
| NIX-BH3 | VVEGEKEVEALKKSADWVSDWS | 23 | VVEGEKEXEALXKSADWVSDWS | 69 |
| 4ICD(ERBB4)-BH3 | SMARDPQRYLVIQGDDRMKL | 24 | SMARDPXRYLXIQGDDRMKL | 70 |

Table 2 lists human sequences which target the BH3 binding site and are implicated in cancers, autoimmune disorders, metabolic diseases and other human disease conditions.

TABLE 3

| | Sequence (bold = | SEQ | Cross-linked Sequence | SEQ |
|---|---|---|---|---|
| Name | critical residues) | ID NO: | (X = x-link residue) | ID NO: |
| P53 peptides | | | | |
| hp53 peptide 1 | LSQETFSDLWKLLPEN | 71 | LSQETFSDXWKLLPEX | 72 |
| hp53 peptide 2 | LSQETFSDLWKLLPEN | 71 | LSQEXFSDLWKXLPEN | 73 |
| hp53 peptide 3 | LSQETFSDLWKLLPEN | 71 | LSQXTFSDLWXLLPEN | 74 |
| hp53 peptide 4 | LSQETFSDLWKLLPEN | 71 | LSQETFXDLWKLLXEN | 75 |
| hp53 peptide 5 | LSQETFSDLWKLLPEN | 71 | QSQQTFXNLWRLLXQN | 76 |

Table 3 lists human sequences which target the p53 binding site of MDM2/X and
are implicated in cancers.

TABLE 4

| | Sequence (bold = | SEQ | Cross-linked Sequence | SEQ |
|---|---|---|---|---|
| Name | critical residues) | ID NO: | (X = x-link residue) | ID NO: |
| GPCR peptide ligand | | | | |
| Angiotensin II | DRVYIHPF | 77 | DRXYXHPF | 83 |
| Bombesin | EQRLGNQWAVGHLM | 78 | EQRLGNXWAVGHLX | 84 |
| Bradykinin | RPPGFSPFR | 79 | RPPXFSPFRX | 85 |
| C5a | ISHKDMQLGR | 80 | ISHKDMXLGRX | 86 |
| C3a | ARASHLGLAR | 81 | ARASHLXLARX | 87 |
| α-melanocyte | SYSMEHFRWGKPV | 82 | SYSMXHFRWXKPV | 88 |
| stimulating hormone | | | | |

Table 4 lists sequences which target human G protein-coupled receptors and are implicated in numerous human disease conditions (Tyndall et al. (2005), Chem. Rev. 105:793-826).

### Peptidomimetic Macrocycles of the Invention

In some embodiments, the peptidomimetic macrocycles of the invention have the Formula (I):

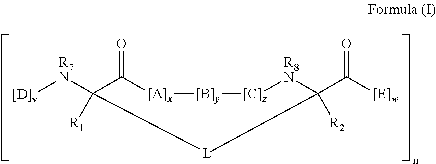

wherein:

each A, C, D, and E is independently a natural or non-natural amino acid;

B is a natural or non-natural amino acid, amino acid analog,

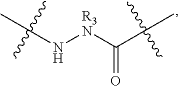

[—NH-L₃-CO—], [—NH-L₃-SO₂—], or [—NH-L₃-];

R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-;

R₃ is hydrogen, alkyl, alkenyl, alkynyl, arylalkyl, heteroalkyl, cycloalkyl, heterocycloalkyl, cycloalkylalkyl, cycloaryl, or heterocycloaryl, optionally substituted with R₅;

L is a macrocycle-forming linker of the formula -L₁-L₂-; L₁ and L₂ are independently alkylene, alkenylene, alkynylene, heteroalkylene, cycloalkylene, heterocycloalkylene, cycloarylene, heterocycloarylene, or [—R₄—K—R₄-]ₙ, each being optionally substituted with R₅;

each R₄ is alkylene, alkenylene, alkynylene, heteroalkylene, cycloalkylene, heterocycloalkylene, arylene, or heteroarylene;

each K is O, S, SO, SO₂, CO, CO₂, or CONR₃;

each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;

each R₆ is independently —H, alkyl, alkenyl, alkynyl, arylalkyl, cycloalkylalkyl, heterocycloalkyl, a fluorescent moiety, a radioisotope or a therapeutic agent;

R₇ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with a D residue;

R₈ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with an E residue;

each of v and w is independently an integer from 1-1000;

each of x, y, and z is independently an integer from 0-10; u is an integer from 1-10; and

n is an integer from 1-5.

In one example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl.

In some embodiments of the invention, x+y+z is at least 3. In other embodiments of the invention, x+y+z is 1, 2, 3, 4, 5, 6, 7, 8, 9 or 10. Each occurrence of A, B, C, D or E in a macrocycle or macrocycle precursor of the invention is independently selected. For example, a sequence represented by the formula [A]ₓ, when x is 3, encompasses embodiments where the amino acids are not identical, e.g. Gln-Asp-Ala as well as embodiments where the amino acids are identical, e.g. Gln-Gln-Gln. This applies for any value of x, y, or z in the indicated ranges.

In some embodiments, the peptidomimetic macrocycle of the invention comprises a secondary structure which is an α-helix and R₈ is —H, allowing intrahelical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

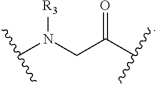

In other embodiments, the length of the macrocycle-forming linker L as measured from a first Ca to a second Cα is selected to stabilize a desired secondary peptide structure, such as an α-helix formed by residues of the peptidomimetic macrocycle including, but not necessarily limited to, those between the first Cα to a second Cα.

In one embodiment, the peptidomimetic macrocycle of Formula (I) is:

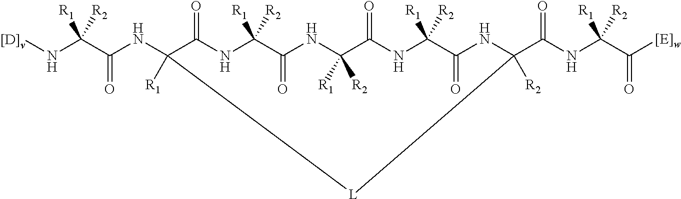

wherein each R₁ and R₂ is independently independently —H, alkyl, alkenyl, alkynyl, arylalkyl, cycloalkyl, cycloalkylalkyl, heteroalkyl, or heterocycloalkyl, unsubstituted or substituted with halo-.

In related embodiments, the peptidomimetic macrocycle of Formula (I) is:

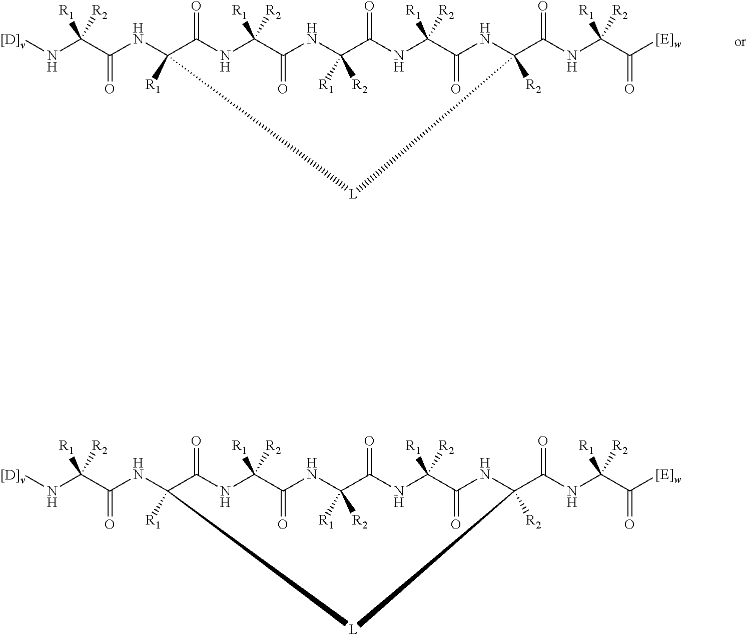

In other embodiments, the peptidomimetic macrocycle of Formula (I) is a compound of any of the formulas shown below:

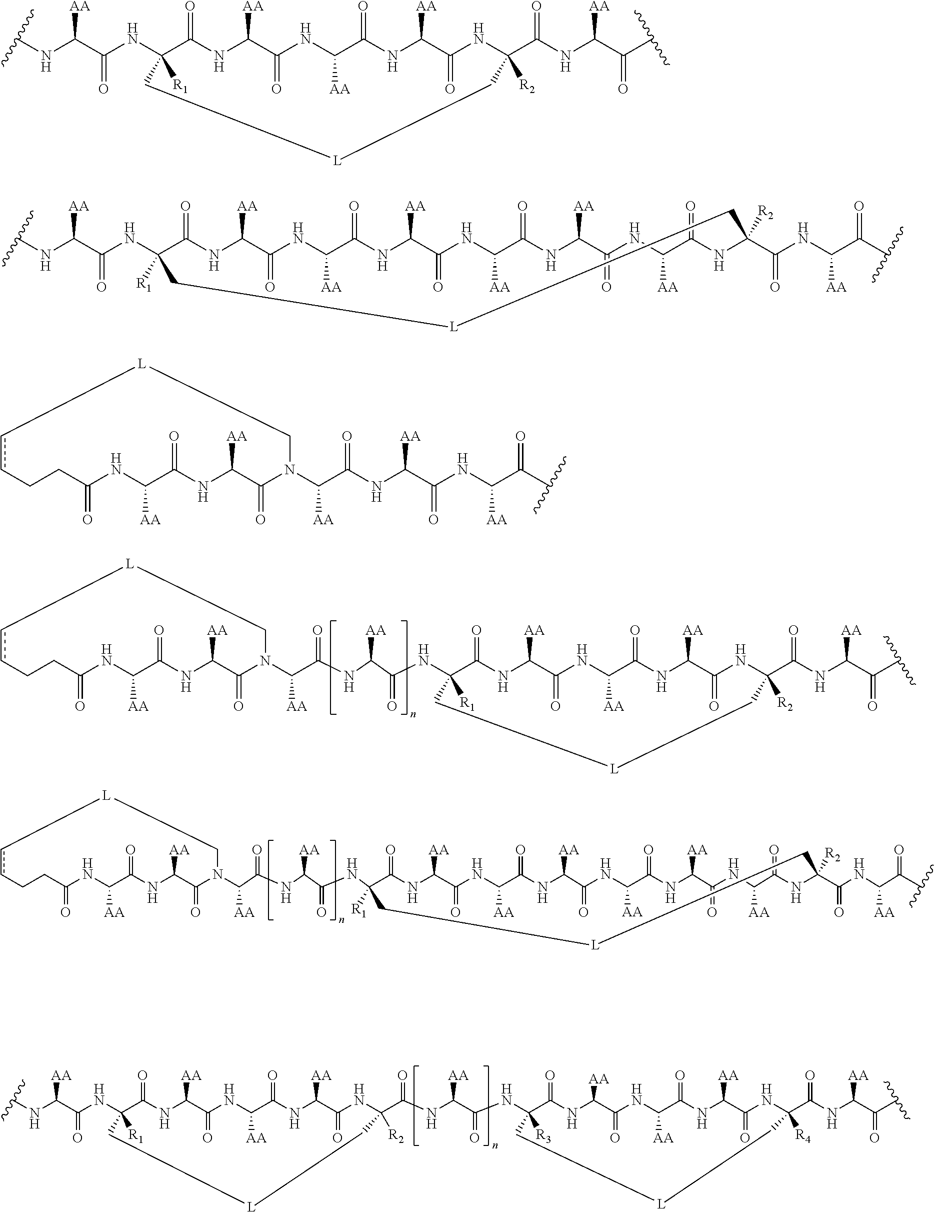

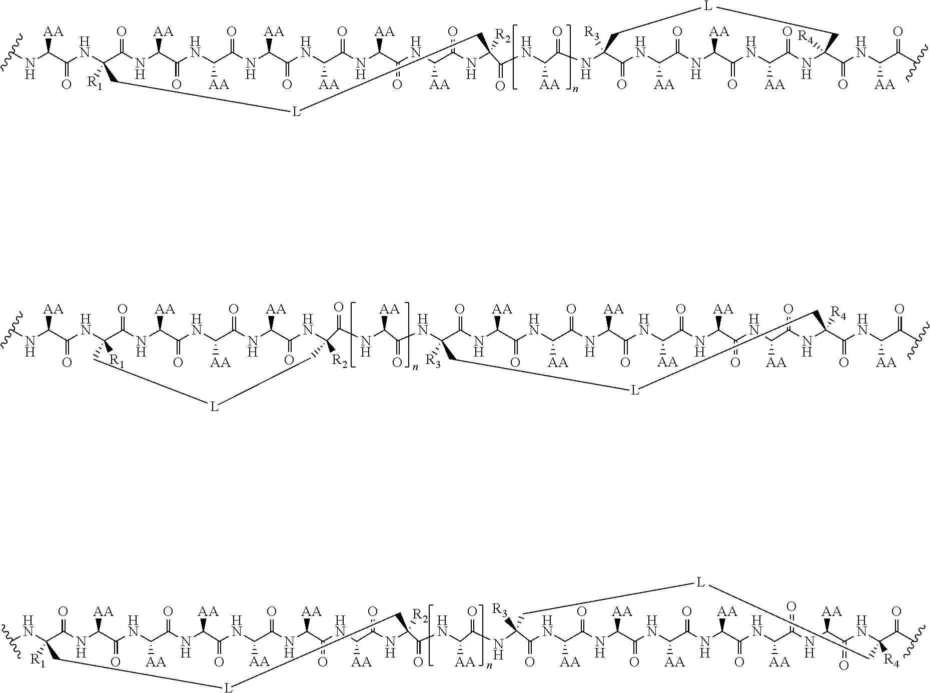

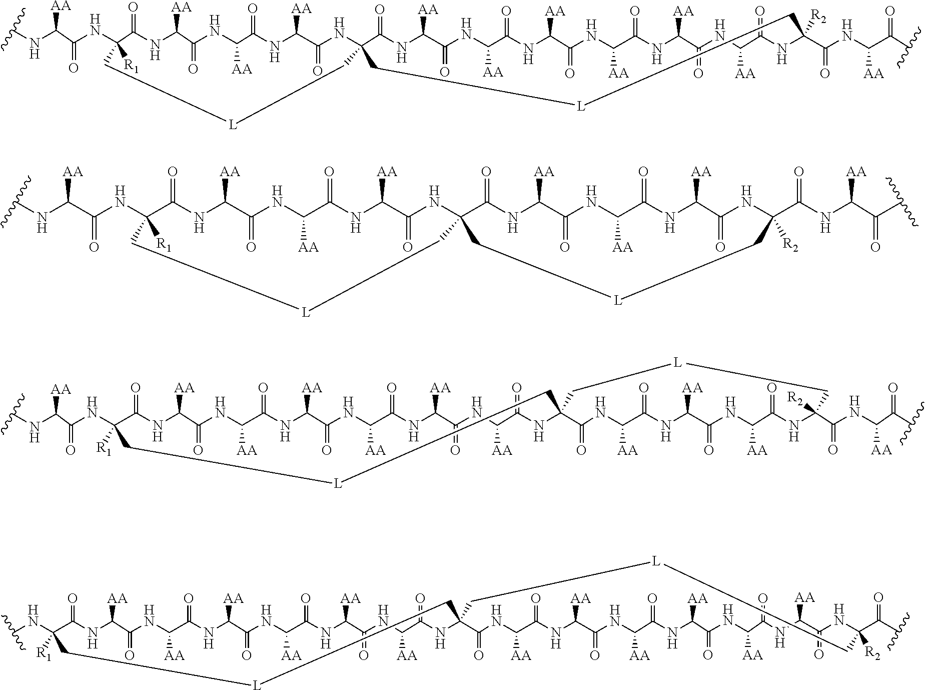

wherein “AA” represents any natural or non-natural amino acid side chain and “ [symbol] ” is [D]ᵥ, [E]_{w} as defined above, and n is an integer between 0 and 20, 50, 100, 200, 300, 400 or 500. In some embodiments, n is 0. In other embodiments, n is less than 50.

Exemplary embodiments of the macrocycle-forming linker L are shown below.

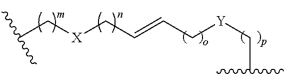

where X, Y=—CH₂—, O, S, or NH
m, n, o, p=0-10

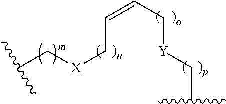

where X,Y=—CH₂—, O, S, or NH
m, n, o, p=0-10

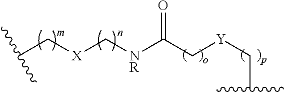

where X, Y=—CH₂—, O, S, or NH
m, n, o, p=0-10
R=H, alkyl, other substituent

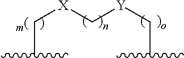

where X, Y=—CH₂—, O, S, or NH
m, n, o=0-10

Exemplary embodiments of peptidomimetic macrocycles of the invention are shown below (SEQ ID NOS: 89-90, respectively, in order of appearance):

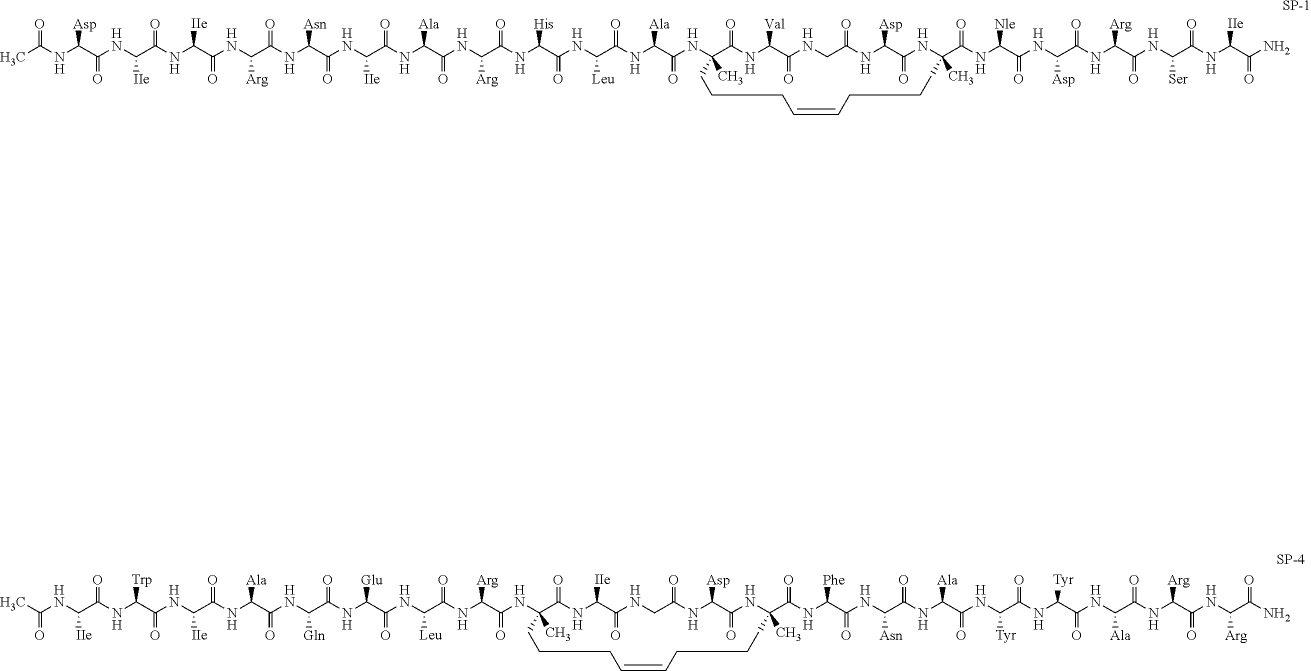

Other embodiments of peptidomimetic macrocycles of the invention include analogs of the macrocycles shown above.

In some embodiments, the peptidomimetic macrocycles of the invention have the Formula (II):

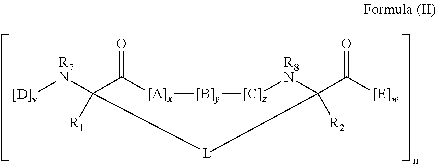

wherein:

each A, C, D, and E is independently a natural or non-natural amino acid;

B is a natural or non-natural amino acid, amino acid analog,

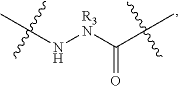

[—NH-L₃-CO—], [—NH-L₃-SO₂—], or [—NH-L₃-];

R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-;

R₃ is hydrogen, alkyl, alkenyl, alkynyl, arylalkyl, heteroalkyl, cycloalkyl, heterocycloalkyl, cycloalkylalkyl, cycloaryl, or heterocycloaryl, optionally substituted with R₅;

L is a macrocycle-forming linker of the formula

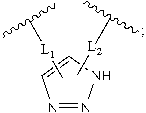

L₁, L₂ and L₃ are independently alkylene, alkenylene, alkynylene, heteroalkylene, cycloalkylene, heterocycloalkylene, cycloarylene, heterocycloarylene, or [—R₄—K—R₄-]ₙ, each being optionally substituted with R₅;

each R₄ is alkylene, alkenylene, alkynylene, heteroalkylene, cycloalkylene, heterocycloalkylene, arylene, or heteroarylene;

each K is O, S, SO, SO₂, CO, CO₂, or CONR₃;

each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;

each R₆ is independently —H, alkyl, alkenyl, alkynyl, arylalkyl, cycloalkylalkyl, heterocycloalkyl, a fluorescent moiety, a radioisotope or a therapeutic agent;

R₇ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with a D residue;

R₈ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with an E residue;

each of v and w is independently an integer from 1-1000;

each of x, y, and z is independently an integer from 0-10; u is an integer from 1-10; and

n is an integer from 1-5.

In one example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl.

In some embodiments of the invention, x+y+z is at least 3. In other embodiments of the invention, x+y+z is 1, 2, 3, 4, 5, 6, 7, 8, 9 or 10. Each occurrence of A, B, C, D or E in a macrocycle or macrocycle precursor of the invention is independently selected. For example, a sequence represented by the formula [A]ₓ, when x is 3, encompasses embodiments where the amino acids are not identical, e.g. Gln-Asp-Ala as well as embodiments where the amino acids are identical, e.g. Gln-Gln-Gln. This applies for any value of x, y, or z in the indicated ranges.

In some embodiments, the peptidomimetic macrocycle of the invention comprises a secondary structure which is an α-helix and R₈ is —H, allowing intrahelical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

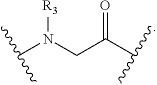

In other embodiments, the length of the macrocycle-forming linker L as measured from a first Ca to a second Cα is selected to stabilize a desired secondary peptide structure, such as an α-helix formed by residues of the peptidomimetic macrocycle including, but not necessarily limited to, those between the first Cα to a second Cα.

Exemplary embodiments of the macrocycle-forming linker L are shown below.

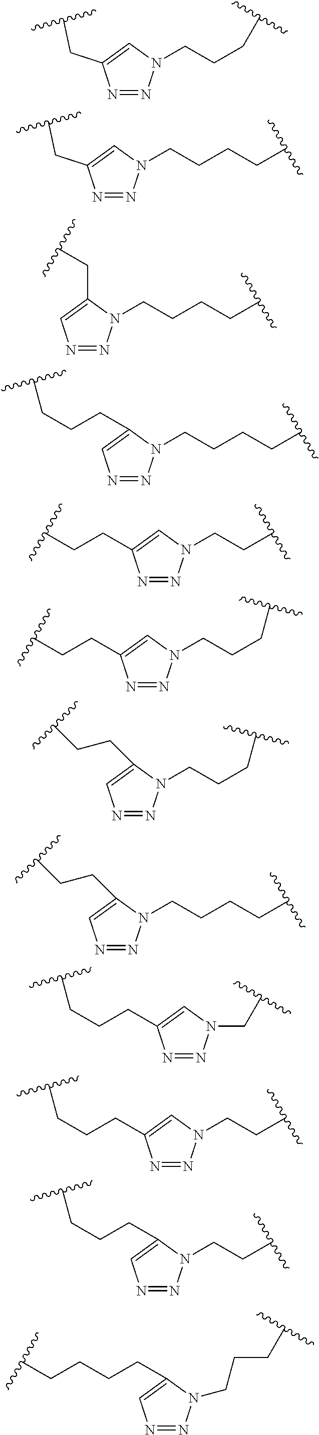

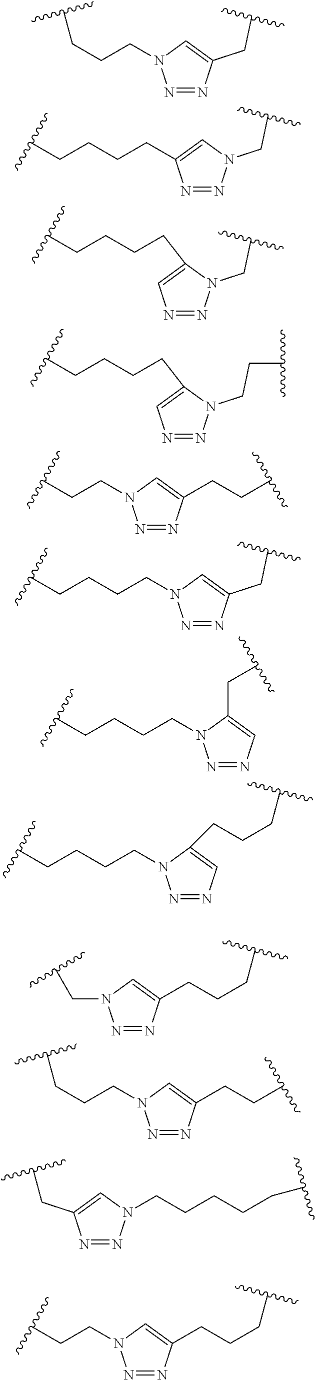

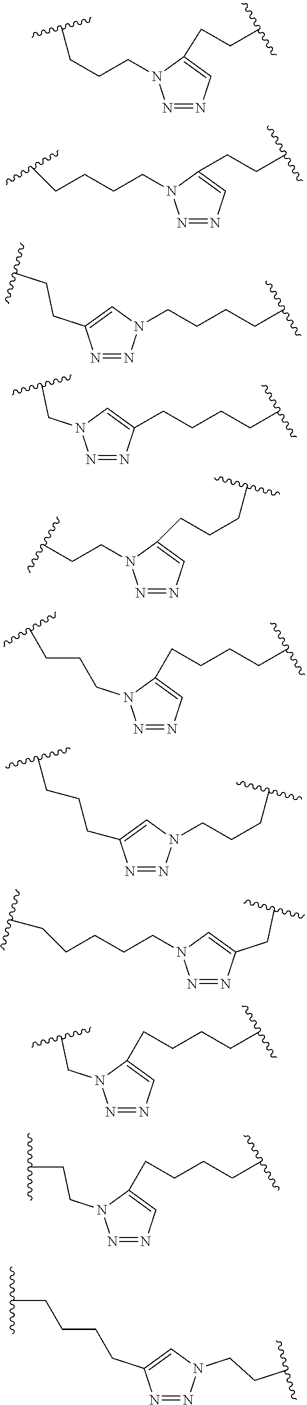

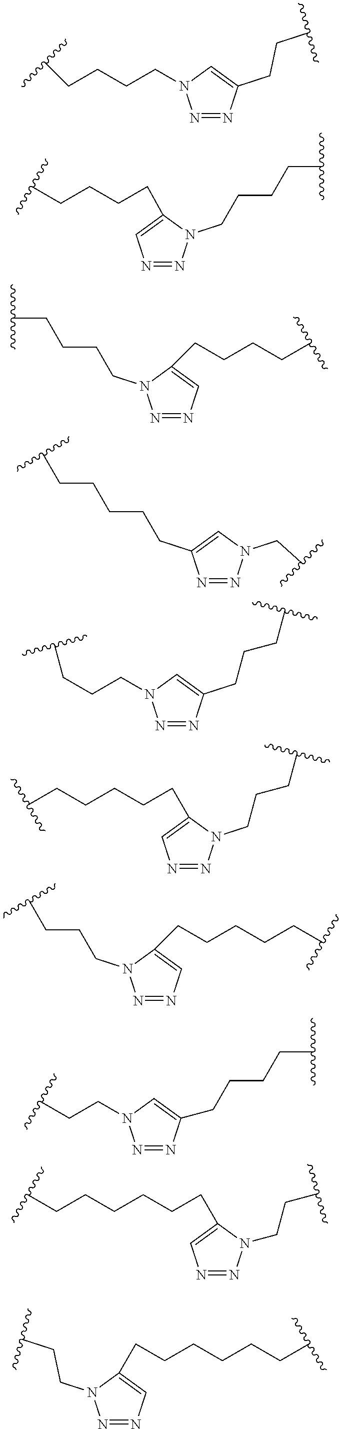

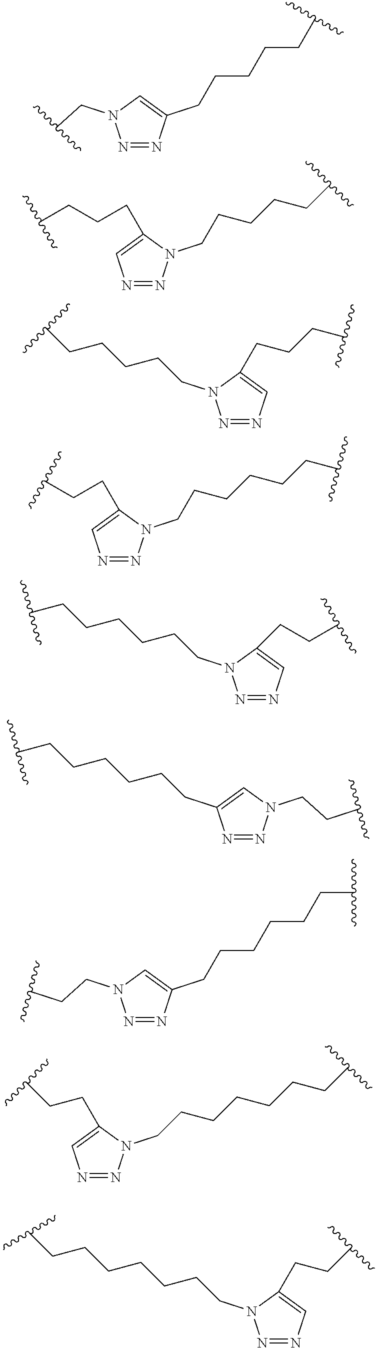

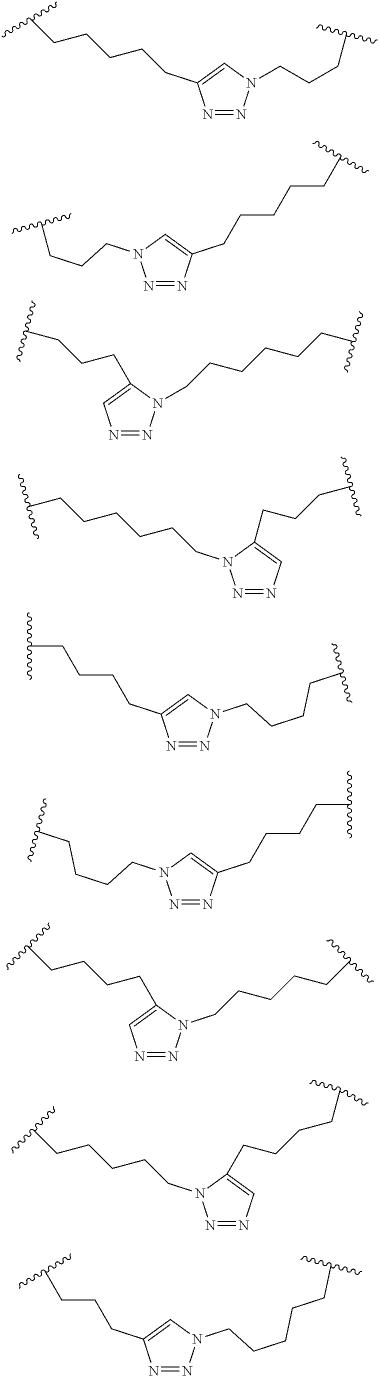

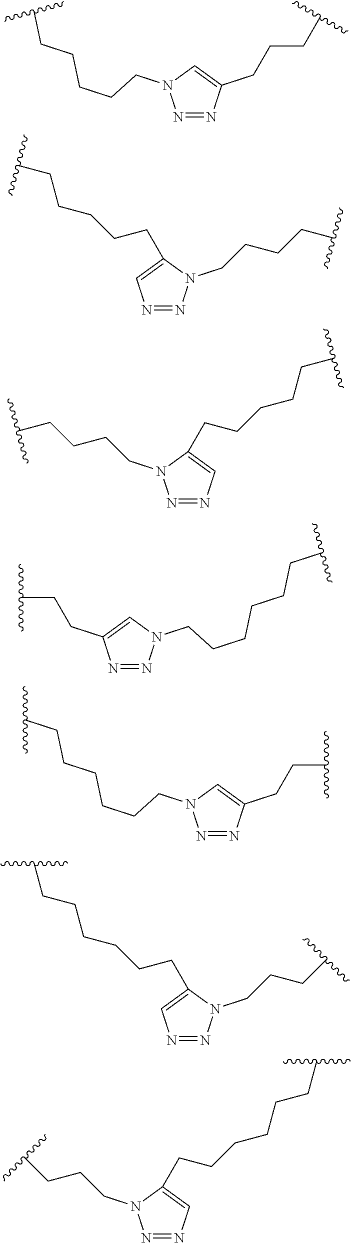

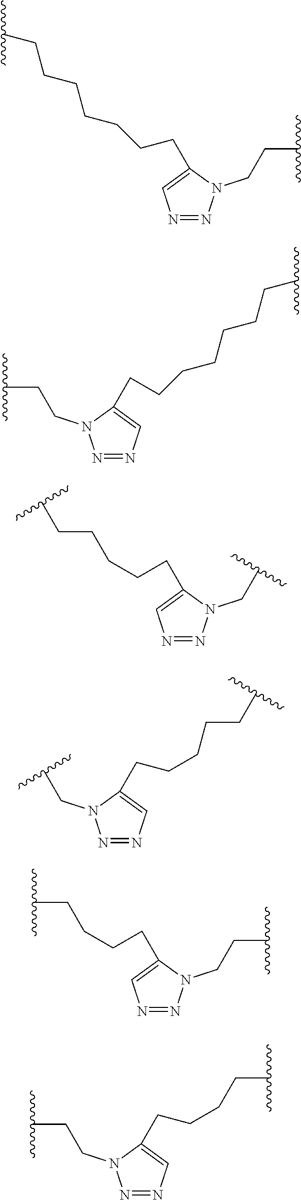 [SMILES: *CCCCCCCc1cnnn1CC* ; *CCCCCCCc1cnnn1CC* ; *CCCCCc1cnnn1C* ; *CCCCCc1cnnn1C* ; *CCCCc1cnnn1CC* ; *CCCCc1cnnn1CC*]

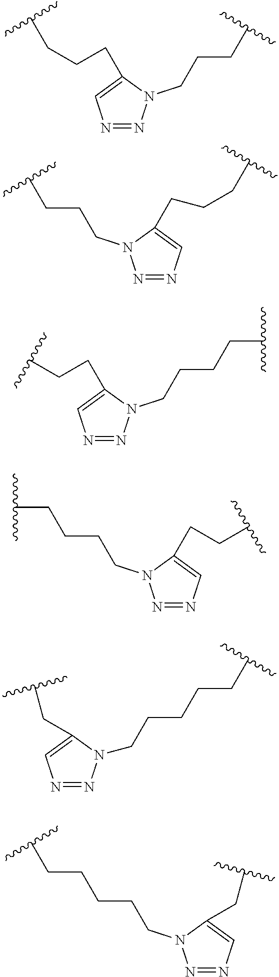 [SMILES: *CCCc1cnnn1CCC* ; *CCCc1cnnn1CCC* ; *CCCCn1nncc1CC* ; *CCCCn1nncc1CC* ; *CCCCCn1nncc1C* ; *CCCCCn1nncc1C*]

In other embodiments, the invention provides peptidomimetic macrocycles of Formula (III):

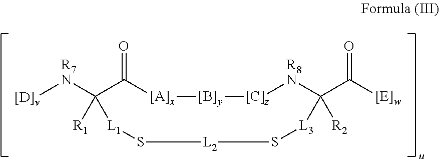

wherein:
each A, C, D, and E is independently a natural or non-natural amino acid;
B is a natural or non-natural amino acid, amino acid analog,

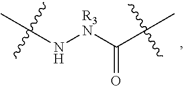

[—NH-L₄-CO—], [—NH-L₄-SO₂—], or [—NH-L₄-];
R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-;
R₃ is hydrogen, alkyl, alkenyl, alkynyl, arylalkyl, heteroalkyl, cycloalkyl, heterocycloalkyl, cycloalkylalkyl, cycloaryl, or heterocycloaryl, unsubstituted or substituted with R₅;
L₁, L₂, L₃ and L₄ are independently alkylene, alkenylene, alkynylene, heteroalkylene, cycloalkylene, heterocycloalkylene, cycloarylene, heterocycloarylene or [—R₄—K—R₄-]n, each being unsubstituted or substituted with R₅;
K is O, S, SO, SO₂, CO, CO₂, or CONR₃;
each R₄ is alkylene, alkenylene, alkynylene, heteroalkylene, cycloalkylene, heterocycloalkylene, arylene, or heteroarylene;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
R₇ is —H, alkyl, alkenyl, alkynyl, arylalkyl, cycloalkyl, heteroalkyl, cycloalkylalkyl, heterocycloalkyl, cycloaryl, or heterocycloaryl, unsubstituted or substituted with R₅, or part of a cyclic structure with a D residue;
R₈ is —H, alkyl, alkenyl, alkynyl, arylalkyl, cycloalkyl, heteroalkyl, cycloalkylalkyl, heterocycloalkyl, cycloaryl, or heterocycloaryl, unsubstituted or substituted with R₅, or part of a cyclic structure with an E residue;
each of v and w is independently an integer from 1-1000;
each of x, y, and z is independently an integer from 0-10; u is an integer from 1-10; and
n is an integer from 1-5.

In one example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl.

In some embodiments of the invention, x+y+z is at least 3. In other embodiments of the invention, x+y+z is 3, 4, 5, 6, 7, 8, 9 or 10. Each occurrence of A, B, C, D or E in a macrocycle or macrocycle precursor of the invention is independently selected. For example, a sequence represented by the formula [A]ₓ, when x is 3, encompasses embodiments where the amino acids are not identical, e.g. Gln-Asp-Ala as well as embodiments where the amino acids are identical, e.g. Gln-Gln-Gln. This applies for any value of x, y, or z in the indicated ranges.

In some embodiments, the peptidomimetic macrocycle of the invention comprises a secondary structure which is an α-helix and R₈ is —H, allowing intrahelical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

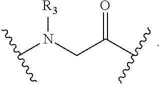

In other embodiments, the length of the macrocycle-forming linker [-L₁-S-L₂-S-L₃-] as measured from a first Cα to a second Cα is selected to stabilize a desired secondary peptide structure, such as an α-helix formed by residues of the peptidomimetic macrocycle including, but not necessarily limited to, those between the first Cα to a second Cα.

Macrocycles or macrocycle precursors are synthesized, for example, by solution phase or solid-phase methods, and can contain both naturally-occurring and non-naturally-occurring amino acids. See, for example, Hunt, “The Non-Protein Amino Acids” in Chemistry and Biochemistry of the Amino Acids, edited by G. C. Barrett, Chapman and Hall, 1985. In some embodiments, the thiol moieties are the side chains of the amino acid residues L-cysteine, D-cysteine, α-methyl-L cysteine, α-methyl-D-cysteine, L-homocysteine, D-homocysteine, α-methyl-L-homocysteine or α-methyl-D-homocysteine. A bis-alkylating reagent is of the general formula X-L₂-Y wherein L₂ is a linker moiety and X and Y are leaving groups that are displaced by —SH moieties to form bonds with L₂. In some embodiments, X and Y are halogens such as I, Br, or Cl.

In other embodiments, D and/or E in the compound of Formula I, II or III are further modified in order to facilitate cellular uptake. In some embodiments, lipidating or PEGylating a peptidomimetic macrocycle facilitates cellular uptake, increases bioavailability, increases blood circulation, alters pharmacokinetics, decreases immunogenicity and/or decreases the needed frequency of administration.

In other embodiments, at least one of [D] and [E] in the compound of Formula I, II or III represents a moiety comprising an additional macrocycle-forming linker such that the peptidomimetic macrocycle comprises at least two macrocycle-forming linkers. In a specific embodiment, a peptidomimetic macrocycle comprises two macrocycle-forming linkers.

In the peptidomimetic macrocycles of the invention, any of the macrocycle-forming linkers described herein may be used in any combination with any of the sequences shown in Tables 1-4 and also with any of the R- substituents indicated herein.

In some embodiments, the peptidomimetic macrocycle comprises at least one α-helix motif. For example, A, B and/or C in the compound of Formula I, II or III include one or more α-helices. As a general matter, α-helices include between 3 and 4 amino acid residues per turn. In some embodiments, the α-helix of the peptidomimetic macrocycle includes 1 to 5 turns and, therefore, 3 to 20 amino acid residues. In specific embodiments, the α-helix includes 1 turn, 2 turns, 3 turns, 4 turns, or 5 turns. In some embodiments, the macrocycle-forming linker stabilizes an α-helix motif included within the peptidomimetic macrocycle. Thus, in some embodiments, the length of the macrocycle-forming linker L from a first Cα to a second Cα is selected to increase the stability of an α-helix. In some embodiments, the macrocycle-forming linker spans from 1 turn to 5 turns of the α-helix. In some embodiments, the macrocycle-forming linker spans approximately 1 turn, 2 turns, 3 turns, 4 turns, or 5 turns of the α-helix. In some embodiments, the length of the macrocycle-forming linker is approximately 5 Å to 9 Å per turn of the α-helix, or approximately 6 Å to 8 Å per turn of the α-helix. Where the macrocycle-forming linker spans approximately 1 turn of an α-helix, the length is equal to approximately 5 carbon-carbon bonds to 13 carbon-carbon bonds, approximately 7 carbon-carbon bonds to 11 carbon-carbon bonds, or approximately 9 carbon-carbon bonds. Where the macrocycle-forming linker spans approximately 2 turns of an α-helix, the length is equal to approximately 8 carbon-carbon bonds to 16 carbon-carbon bonds, approximately 10 carbon-carbon bonds to 14 carbon-carbon bonds, or approximately 12 carbon-carbon bonds. Where the macrocycle-forming linker spans approximately 3 turns of an α-helix, the length is equal to approximately 14 carbon-carbon bonds to 22 carbon-carbon bonds, approximately 16 carbon-carbon bonds to 20 carbon-carbon bonds, or approximately 18 carbon-carbon bonds. Where the macrocycle-forming linker spans approximately 4 turns of an α-helix, the length is equal to approximately 20 carbon-carbon bonds to 28 carbon-carbon bonds, approximately 22 carbon-carbon bonds to 26 carbon-carbon bonds, or approximately 24 carbon-carbon bonds. Where the macrocycle-forming linker spans approximately 5 turns of an α-helix, the length is equal to approximately 26 carbon-carbon bonds to 34 carbon-carbon bonds, approximately 28 carbon-carbon bonds to 32 carbon-carbon bonds, or approximately 30 carbon-carbon bonds. Where the macrocycle-forming linker spans approximately 1 turn of an α-helix, the linkage contains approximately 4 atoms to 12 atoms, approximately 6 atoms to 10 atoms, or approximately 8 atoms. Where the macrocycle-forming linker spans approximately 2 turns of the α-helix, the linkage contains approximately 7 atoms to 15 atoms, approximately 9 atoms to 13 atoms, or approximately 11 atoms. Where the macrocycle-forming linker spans approximately 3 turns of the α-helix, the linkage contains approximately 13 atoms to 21 atoms, approximately 15 atoms to 19 atoms, or approximately 17 atoms. Where the macrocycle-forming linker spans approximately 4 turns of the α-helix, the linkage contains approximately 19 atoms to 27 atoms, approximately 21 atoms to 25 atoms, or approximately 23 atoms. Where the macrocycle-forming linker spans approximately 5 turns of the α-helix, the linkage contains approximately 25 atoms to 33 atoms, approximately 27 atoms to 31 atoms, or approximately 29 atoms. Where the macrocycle-forming linker spans approximately 1 turn of the α-helix, the resulting macrocycle forms a ring containing approximately 17 members to 25 members, approximately 19 members to 23 members, or approximately 21 members. Where the macrocycle-forming linker spans approximately 2 turns of the α-helix, the resulting macrocycle forms a ring containing approximately 29 members to 37 members, approximately 31 members to 35 members, or approximately 33 members. Where the macrocycle-forming linker spans approximately 3 turns of the α-helix, the resulting macrocycle forms a ring containing approximately 44 members to 52 members, approximately 46 members to 50 members, or approximately 48 members. Where the macrocycle-forming linker spans approximately 4 turns of the α-helix, the resulting macrocycle forms a ring containing approximately 59 members to 67 members, approximately 61 members to 65 members, or approximately 63 members. Where the macrocycle-forming linker spans approximately 5 turns of the α-helix, the resulting macrocycle forms a ring containing approximately 74 members to 82 members, approximately 76 members to 80 members, or approximately 78 members.

In other embodiments, the invention provides peptidomimetic macrocycles of Formula (IV) or (IVa):

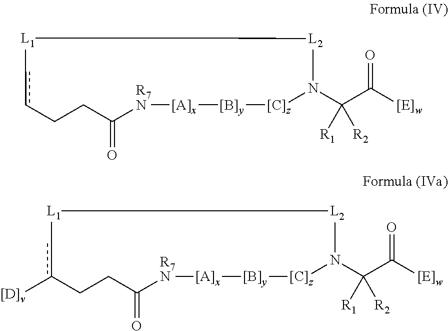

wherein:

each A, C, D, and E is independently a natural or non-natural amino acid;

B is a natural or non-natural amino acid, amino acid analog,

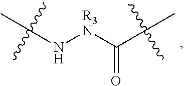

[—NH-L₃-CO—], [—NH-L₃-SO₂—], or [—NH-L₃-];

R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-, or part of a cyclic structure with an E residue;

R₃ is hydrogen, alkyl, alkenyl, alkynyl, arylalkyl, heteroalkyl, cycloalkyl, heterocycloalkyl, cycloalkylalkyl, cycloaryl, or heterocycloaryl, optionally substituted with R₅;

L is a macrocycle-forming linker of the formula -L₁-L₂-;

L₁ and L₂ are independently alkylene, alkenylene, alkynylene, heteroalkylene, cycloalkylene, heterocycloalkylene, cycloarylene, heterocycloarylene, or [—R₄—K—R₄-]ₙ, each being optionally substituted with R₅;

each R₄ is alkylene, alkenylene, alkynylene, heteroalkylene, cycloalkylene, heterocycloalkylene, arylene, or heteroarylene;

each K is O, S, SO, SO₂, CO, CO₂, or CONR₃;

each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;

each R₆ is independently —H, alkyl, alkenyl, alkynyl, arylalkyl, cycloalkylalkyl, heterocycloalkyl, a fluorescent moiety, a radioisotope or a therapeutic agent;

R₇ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅;

v is an integer from 1-1000;

w is an integer from 1-1000;

x is an integer from 0-10;

y is an integer from 0-10;

z is an integer from 0-10; and

n is an integer from 1-5.

In one example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl.

In some embodiments of the invention, x+y+z is at least 3. In other embodiments of the invention, x+y+z is 1, 2, 3, 4, 5, 6, 7, 8, 9 or 10. Each occurrence of A, B, C, D or E in a macrocycle or macrocycle precursor of the invention is independently selected. For example, a sequence represented by the formula [A]ₓ, when x is 3, encompasses embodiments where the amino acids are not identical, e.g. Gln-Asp-Ala as well as embodiments where the amino acids are identical, e.g. Gln-Gln-Gln. This applies for any value of x, y, or z in the indicated ranges.

In some embodiments, the peptidomimetic macrocycle of the invention comprises a secondary structure which is an α-helix and R₈ is —H, allowing intrahelical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

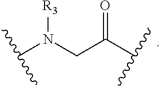

In other embodiments, the length of the macrocycle-forming linker L as measured from a first Ca to a second Cα is selected to stabilize a desired secondary peptide structure, such as an α-helix formed by residues of the peptidomimetic macrocycle including, but not necessarily limited to, those between the first Cα to a second Cα.

Exemplary embodiments of the macrocycle-forming linker L are shown below.

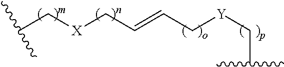

where X, Y=—CH₂—, O, S, or NH
m, n, o, p=0-10

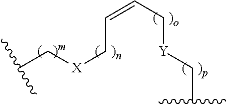

where X, Y=—CH₂—, O, S, or NH
m, n, o, p=0-10

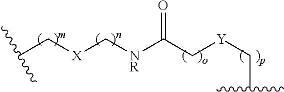

where X, Y=—CH₂—, O, S, or NH
m, n, o, p=0-10
R=H, alkyl, other substituent

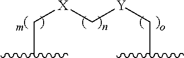

where X, Y=—CH₂—, O, S, or NH
m, n, o=0-10

Preparation of Peptidomimetic Macrocycles

Peptidomimetic macrocycles of the invention may be prepared by any of a variety of methods known in the art. For example, any of the residues indicated by “X” in Tables 1, 2, 3 or 4 may be substituted with a residue capable of forming a crosslinker with a second residue in the same molecule or a precursor of such a residue.

Various methods to effect formation of peptidomimetic macrocycles are known in the art. For example, the preparation of peptidomimetic macrocycles of Formula I is described in Schafmeister et al., J. Am. Chem. Soc. 122:5891-5892 (2000); Schafmeister & Verdine, J. Am. Chem. Soc. 122:5891 (2005); Walensky et al., Science 305:1466-1470 (2004); and U.S. Pat. No. 7,192,713. The α,α-disubstituted amino acids and amino acid precursors disclosed in the cited references may be employed in synthesis of the peptidomimetic macrocycle precursor polypeptides. Following incorporation of such amino acids into precursor polypeptides, the terminal olefins are reacted with a metathesis catalyst, leading to the formation of the peptidomimetic macrocycle.

In other embodiments, the peptidomimetic macrocyles of the invention are of Formula IV or IVa. Methods for the preparation of such macrocycles are described, for example, in U.S. Pat. No. 7,202,332.

In some embodiments, the synthesis of these peptidomimetic macrocycles involves a multi-step process that features the synthesis of a peptidomimetic precursor containing an azide moiety and an alkyne moiety; followed by contacting the peptidomimetic precursor with a macrocyclization reagent to generate a triazole-linked peptidomimetic macrocycle. Macrocycles or macrocycle precursors are synthesized, for example, by solution phase or solid-phase methods, and can contain both naturally-occurring and non-naturally-occurring amino acids. See, for example, Hunt, “The Non-Protein Amino Acids” in Chemistry and Biochemistry of the Amino Acids, edited by G. C. Barrett, Chapman and Hall, 1985.

In some embodiments, an azide is linked to the α-carbon of a residue and an alkyne is attached to the α-carbon of another residue. In some embodiments, the azide moieties are azido-analogs of amino acids L-lysine, D-lysine, alpha-methyl-L-lysine, alpha-methyl-D-lysine, L-ornithine, D-ornithine, alpha-methyl-L-ornithine or alpha-methyl-D-ornithine. In other embodiments, the azide moiety is 2-amino-7-azido-2-methylheptanoic acid or 2-amino-6-azido-2-methylhexanoic acid. In another embodiment, the alkyne moiety is L-propargylglycine. In yet other embodiments, the alkyne moiety is an amino acid selected from the group consisting of L-propargylglycine, D-propargylglycine, (S)-2-amino-2-methyl-4-pentynoic acid, (R)-2-amino-2-methyl-4-pentynoic acid, (S)-2-amino-2-methyl-5-hexynoic acid, (R)-2-amino-2-methyl-5-hexynoic acid, (S)-2-amino-2-methyl-6-heptynoic acid, (R)-2-amino-2-methyl-6-heptynoic acid, (S)-2-amino-2-methyl-7-octynoic acid, (R)-2-amino-2-methyl-7-octynoic acid, (S)-2-amino-2-methyl-8-nonynoic acid and (R)-2-amino-2-methyl-8-nonynoic acid.

In some embodiments, the invention provides a method for synthesizing a peptidomimetic macrocycle, the method comprising the steps of contacting a peptidomimetic precursor of Formula V or Formula VI:

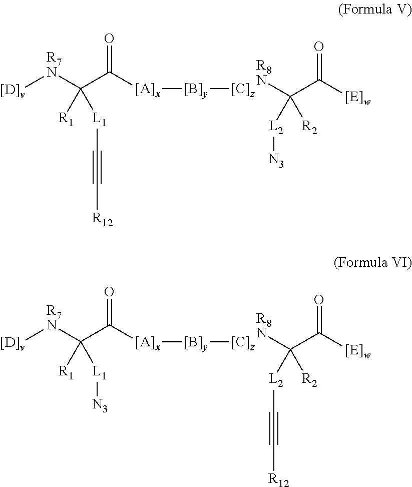

with a macrocyclization reagent;

wherein v, w, x, y, z, A, B, C, D, E, R₁, R₂, R₇, R₈, L₁ and L₂ are as defined for Formula (II); R₁₂ is —H when the macrocyclization reagent is a Cu reagent and R₁₂ is —H or alkyl when the macrocyclization reagent is a Ru reagent; and further wherein said contacting step results in a covalent linkage being formed between the alkyne and azide moiety in Formula III or Formula IV. For example, R₁₂ may be methyl when the macrocyclization reagent is a Ru reagent.

In the peptidomimetic macrocycles of the invention, at least one of R₁ and R₂ is alkyl, alkenyl, alkynyl, arylalkyl, cycloalkyl, cycloalkylalkyl, heteroalkyl, or heterocycloalkyl, unsubstituted or substituted with halo-. In some embodiments, both R₁ and R₂ are independently alkyl, alkenyl, alkynyl, arylalkyl, cycloalkyl, cycloalkylalkyl, heteroalkyl, or heterocycloalkyl, unsubstituted or substituted with halo-. In some embodiments, at least one of A, B, C, D or E is an α,α-disubstituted amino acid. In one example, B is an α,α-disubstituted amino acid. For instance, at least one of A, B, C, D or E is 2-aminoisobutyric acid.

For example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl. The macrocyclization reagent may be a Cu reagent or a Ru reagent.

In some embodiments, the peptidomimetic precursor is purified prior to the contacting step. In other embodiments, the peptidomimetic macrocycle is purified after the contacting step. In still other embodiments, the peptidomimetic macrocycle is refolded after the contacting step. The method may be performed in solution, or, alternatively, the method may be performed on a solid support.

Also envisioned herein is performing the method of the invention in the presence of a target macromolecule that binds to the peptidomimetic precursor or peptidomimetic macrocycle under conditions that favor said binding. In some embodiments, the method is performed in the presence of a target macromolecule that binds preferentially to the peptidomimetic precursor or peptidomimetic macrocycle under conditions that favor said binding. The method may also be applied to synthesize a library of peptidomimetic macrocycles.

In some embodiments, the alkyne moiety of the peptidomimetic precursor of Formula V or Formula VI is a sidechain of an amino acid selected from the group consisting of L-propargylglycine, D-propargylglycine, (S)-2-amino-2-methyl-4-pentynoic acid, (R)-2-amino-2-methyl-4-pentynoic acid, (S)-2-amino-2-methyl-5-hexynoic acid, (R)-2-amino-2-methyl-5-hexynoic acid, (S)-2-amino-2-methyl-6-heptynoic acid, (R)-2-amino-2-methyl-6-heptynoic acid, (S)-2-amino-2-methyl-7-octynoic acid, (R)-2-amino-2-methyl-7-octynoic acid, (S)-2-amino-2-methyl-8-nonynoic acid, and (R)-2-amino-2-methyl-8-nonynoic acid. In other embodiments, the azide moiety of the peptidomimetic precursor of Formula V or Formula VI is a sidechain of an amino acid selected from the group consisting of ε-azido-L-lysine, ε-azido-D-lysine, □ε-azido-α-methyl-L-lysine, ε-azido-α-methyl-D-lysine, δ-azido-α-methyl-L-ornithine, and δ-azido-α-methyl-D-ornithine.

In some embodiments, x+y+z is 3, and A, B and C are independently natural or non-natural amino acids. In other embodiments, x+y+z is 6, and A, B and C are independently natural or non-natural amino acids.

In some embodiments of peptidomimetic macrocycles of the invention, [D]ᵥ and/or [E]_{w} comprise additional peptidomimetic macrocycles or macrocyclic structures. For example, [D]ᵥ may have the formula:

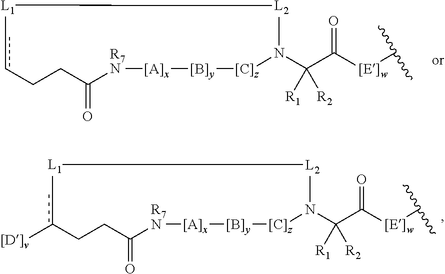

wherein each A, C, D′, and E′ is independently a natural or non-natural amino acid;

B is a natural or non-natural amino acid, amino acid analog,

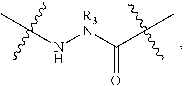

[—NH-L₃-CO—], [—NH-L₃-SO₂—], or [—NH-L₃-];

R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-, or part of a cyclic structure with an E residue;

R₃ is hydrogen, alkyl, alkenyl, alkynyl, arylalkyl, heteroalkyl, cycloalkyl, heterocycloalkyl, cycloalkylalkyl, cycloaryl, or heterocycloaryl, optionally substituted with R₅;

L₁ and L₂ are independently alkylene, alkenylene, alkynylene, heteroalkylene, cycloalkylene, heterocycloalkylene, cycloarylene, heterocycloarylene, or [—R₄—K—R₄-]ₙ, each being optionally substituted with R₅;

each R₄ is alkylene, alkenylene, alkynylene, heteroalkylene, cycloalkylene, heterocycloalkylene, arylene, or heteroarylene;

each K is O, S, SO, SO₂, CO, CO₂, or CONR₃;

each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;

each R₆ is independently —H, alkyl, alkenyl, alkynyl, arylalkyl, cycloalkylalkyl, heterocycloalkyl, a fluorescent moiety, a radioisotope or a therapeutic agent;

R₇ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅;

v is an integer from 1-1000;

w is an integer from 1-1000; and

x is an integer from 0-10.

In another embodiment, [E]_{w} has the formula:

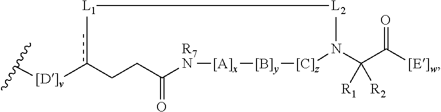

wherein the substituents are as defined in the preceding paragraph.

In some embodiments, the contacting step is performed in a solvent selected from the group consisting of protic solvent, aqueous solvent, organic solvent, and mixtures thereof. For example, the solvent may be chosen from the group consisting of H₂O, THF, THF/H₂O, tBuOH/H₂O, DMF, DIPEA, CH₃CN or CH₂Cl₂, ClCH₂CH₂Cl or a mixture thereof. The solvent may be a solvent which favors helix formation.

Alternative but equivalent protecting groups, leaving groups or reagents are substituted, and certain of the synthetic steps are performed in alternative sequences or orders to produce the desired compounds. Synthetic chemistry transformations and protecting group methodologies (protection and deprotection) useful in synthesizing the compounds described herein include, for example, those such as described in Larock, Comprehensive Organic Transformations, VCH Publishers (1989); Greene and Wuts, Protective Groups in Orgathesis, 2d. Ed., John Wiley and Sons (1991); Fieser and Fieser, Fieser and Fieser's Reagents for Organic Synthesis, John Wiley and Sons (1994); and Paquette, ed., Encyclopedia of Reagents for Organic Synthesis, John Wiley and Sons (1995), and subsequent editions thereof.

The peptidomimetic macrocycles of the invention are made, for example, by chemical synthesis methods, such as described in Fields et al., Chapter 3 in Synthetic Peptides: A User's Guide, ed. Grant, W. H. Freeman & Co., New York, N. Y., 1992, p. 77. Hence, for example, peptides are synthesized using the automated Merrifield techniques of solid phase synthesis with the amine protected by either tBoc or Fmoc chemistry using side chain protected amino acids on, for example, an automated peptide synthesizer (e.g., Applied Biosystems (Foster City, Calif.), Model 430A, 431, or 433).

One manner of producing the peptidomimetic precursors and peptidomimetic macrocycles described herein uses solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but removable by base. Side chain functional groups are protected as necessary with base stable, acid labile groups.

Longer peptidomimetic precursors are produced, for example, by conjoining individual synthetic peptides using native chemical ligation. Alternatively, the longer synthetic peptides are biosynthesized by well known recombinant DNA and protein expression techniques. Such techniques are provided in well-known standard manuals with detailed protocols. To construct a gene encoding a peptidomimetic precursor of this invention, the amino acid sequence is reverse translated to obtain a nucleic acid sequence encoding the amino acid sequence, preferably with codons that are optimum for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

The peptidomimetic precursors are made, for example, in a high-throughput, combinatorial fashion using, for example, a high-throughput polychannel combinatorial synthesizer (e.g., Thuramed TETRAS multichannel peptide synthesizer from CreoSalus, Louisville, Ky. or Model Apex 396 multichannel peptide synthesizer from AAPPTEC, Inc., Louisville, Ky.).

The following synthetic schemes are provided solely to illustrate the present invention and are not intended to limit the scope of the invention, as described herein. To simplify the drawings, the illustrative schemes depict azido amino acid analogs ε-azido-α-methyl-L-lysine and ε-azido-α-methyl-D-lysine, and alkyne amino acid analogs L-propargylglycine, (S)-2-amino-2-methyl-4-pentynoic acid, and (S)-2-amino-2-methyl-6-heptynoic acid. Thus, in the following synthetic schemes, each R₁, R₂, R₇ and R₈ is —H; each L₁ is —(CH₂)₄—; and each L₂ is —(CH₂)—. However, as noted throughout the detailed description above, many other amino acid analogs can be employed in which R₁, R₂, R₇, R₈, L₁ and L₂ can be independently selected from the various structures disclosed herein.

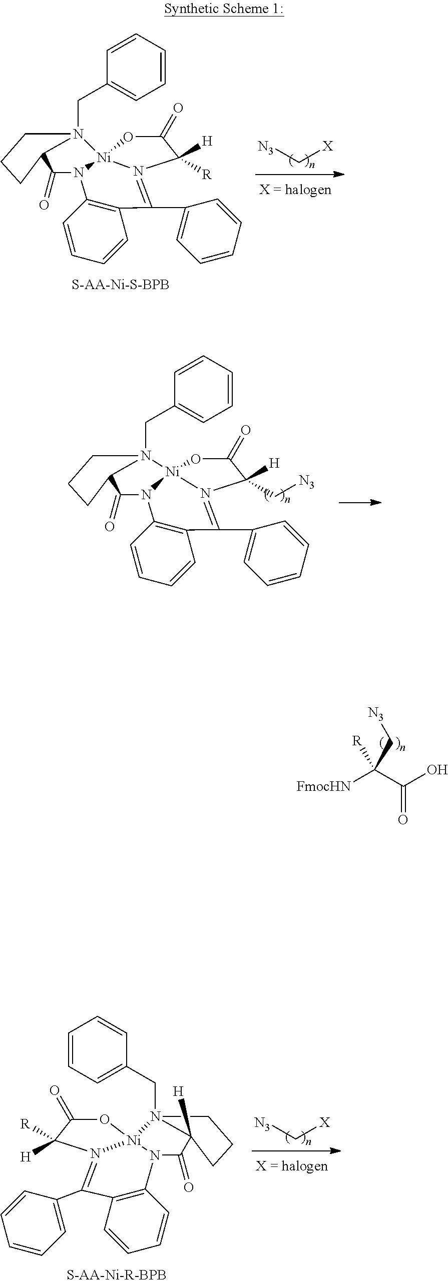

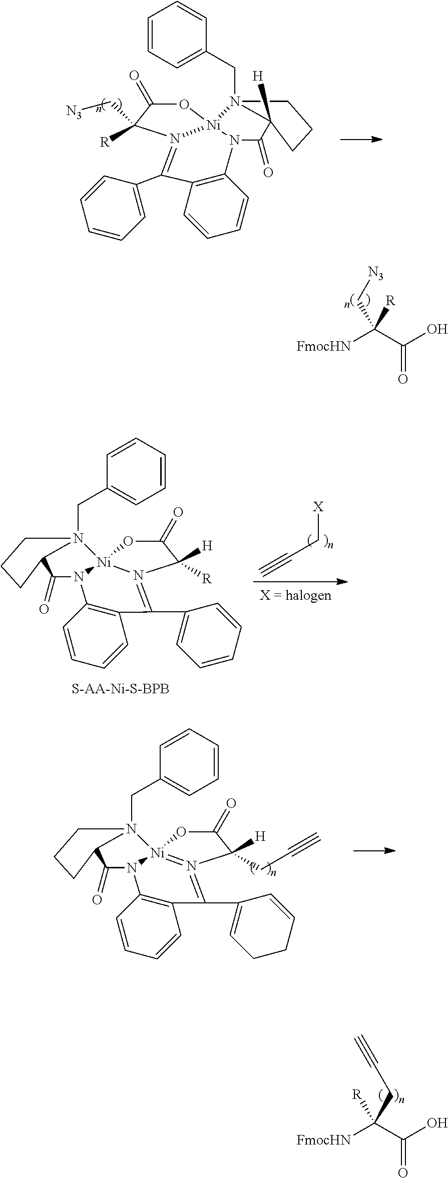

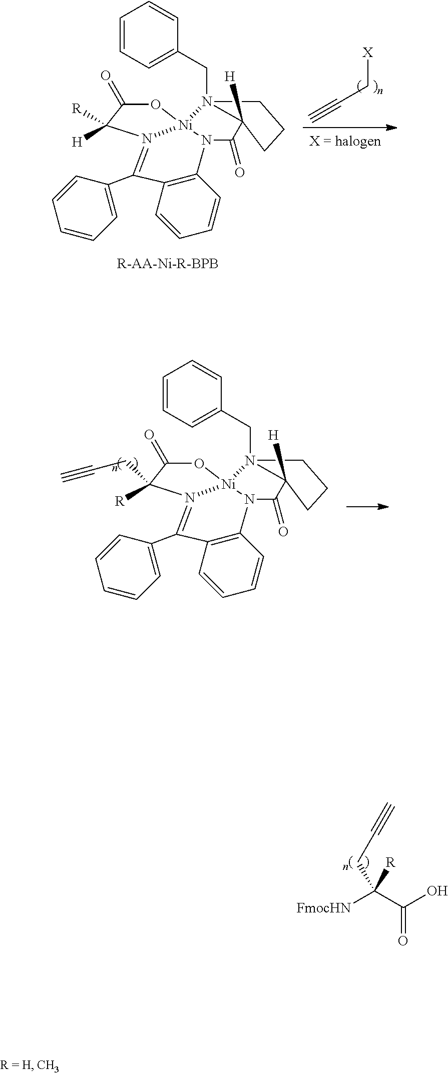

Synthetic Scheme 1 describes the preparation of several compounds of the invention. Ni(II) complexes of Schiff bases derived from the chiral auxiliary (S)-2-[N—(N′-benzylprolyl)amino]benzophenone (BPB) and amino acids such as glycine or alanine are prepared as described in Belokon et al. (1998), Tetrahedron Asymm. 9:4249-4252. The resulting complexes are subsequently reacted with alkylating reagents comprising an azido or alkynyl moiety to yield enantiomerically enriched compounds of the invention. If desired, the resulting compounds can be protected for use in peptide synthesis. In some embodiments of Synthetic Scheme 1, X is iodine.

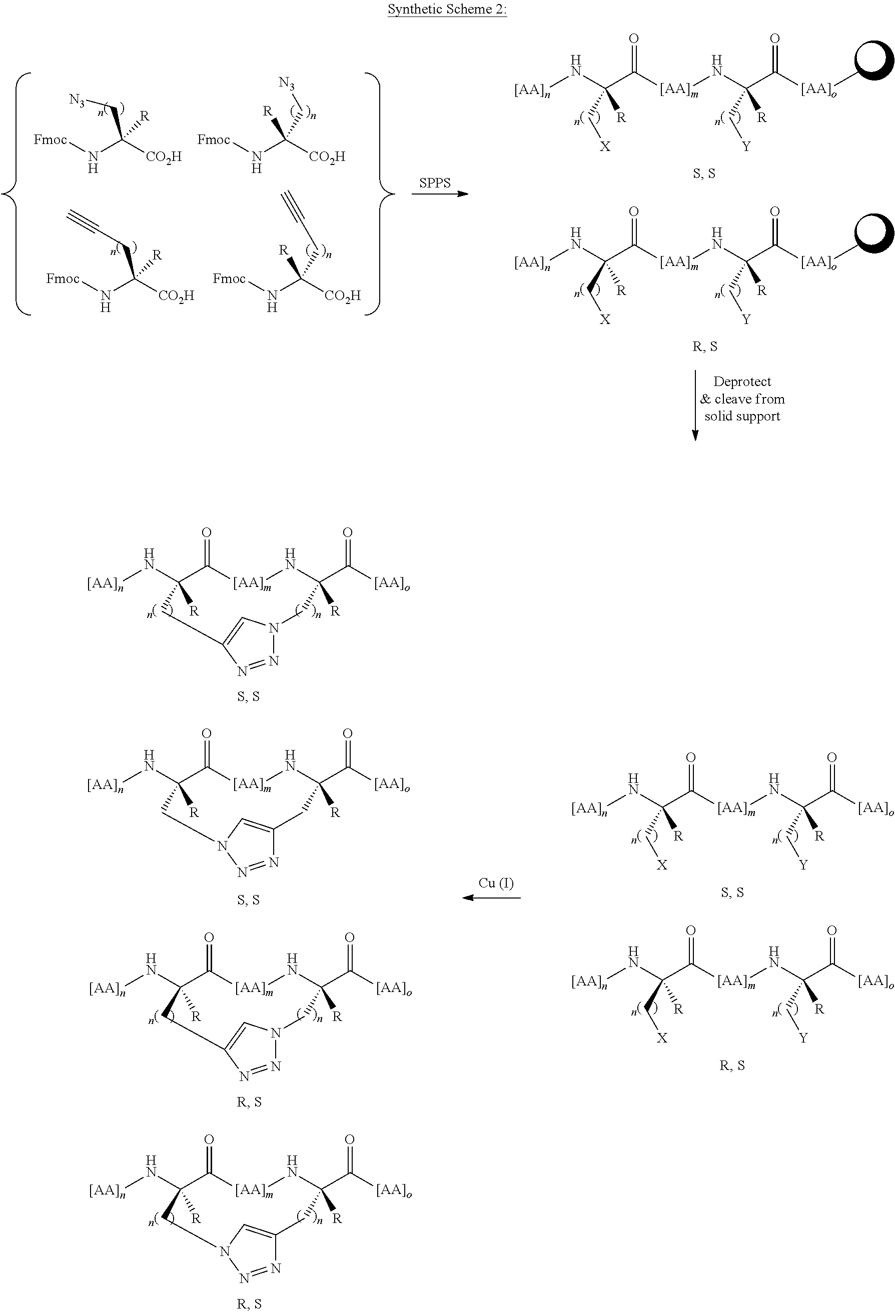

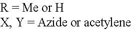

In the general method for the synthesis of peptidomimetic macrocycles shown in Synthetic Scheme 2, the peptidomimetic precursor contains an azide moiety and an alkyne moiety and is synthesized by solution-phase or solid-phase peptide synthesis (SPPS) using the commercially available amino acid N-α-Fmoc-L-propargylglycine and the N-α-Fmoc-protected forms of the amino acids (S)-2-amino-2-methyl-4-pentynoic acid, (S)-2-amino-6-heptynoic acid, (S)-2-amino-2-methyl-6-heptynoic acid, N-methyl-ε-azido-L-lysine, and N-methyl-ε-azido-D-lysine. The peptidomimetic precursor is then deprotected and cleaved from the solid-phase resin by standard conditions (e.g., strong acid such as 95% TFA). The peptidomimetic precursor is reacted as a crude mixture or is purified prior to reaction with a macrocyclization reagent such as a Cu(I) in organic or aqueous solutions (Rostovtsev et al. (2002), Angew. Chem. Int. Ed. 41:2596-2599; Tornoe et al. (2002), J. Org. Chem. 67:3057-3064; Deiters et al. (2003), J. Am. Chem. Soc. 125:11782-11783; Punna et al. (2005), Angew. Chem. Int. Ed. 44:2215-2220). In one embodiment, the triazole forming reaction is performed under conditions that favor α-helix formation. In one embodiment, the macrocyclization step is performed in a solvent chosen from the group consisting of H₂O, THF, CH₃CN, DMF, DIPEA, tBuOH or a mixture thereof. In another embodiment, the macrocyclization step is performed in DMF. In some embodiments, the macrocyclization step is performed in a buffered aqueous or partially aqueous solvent.

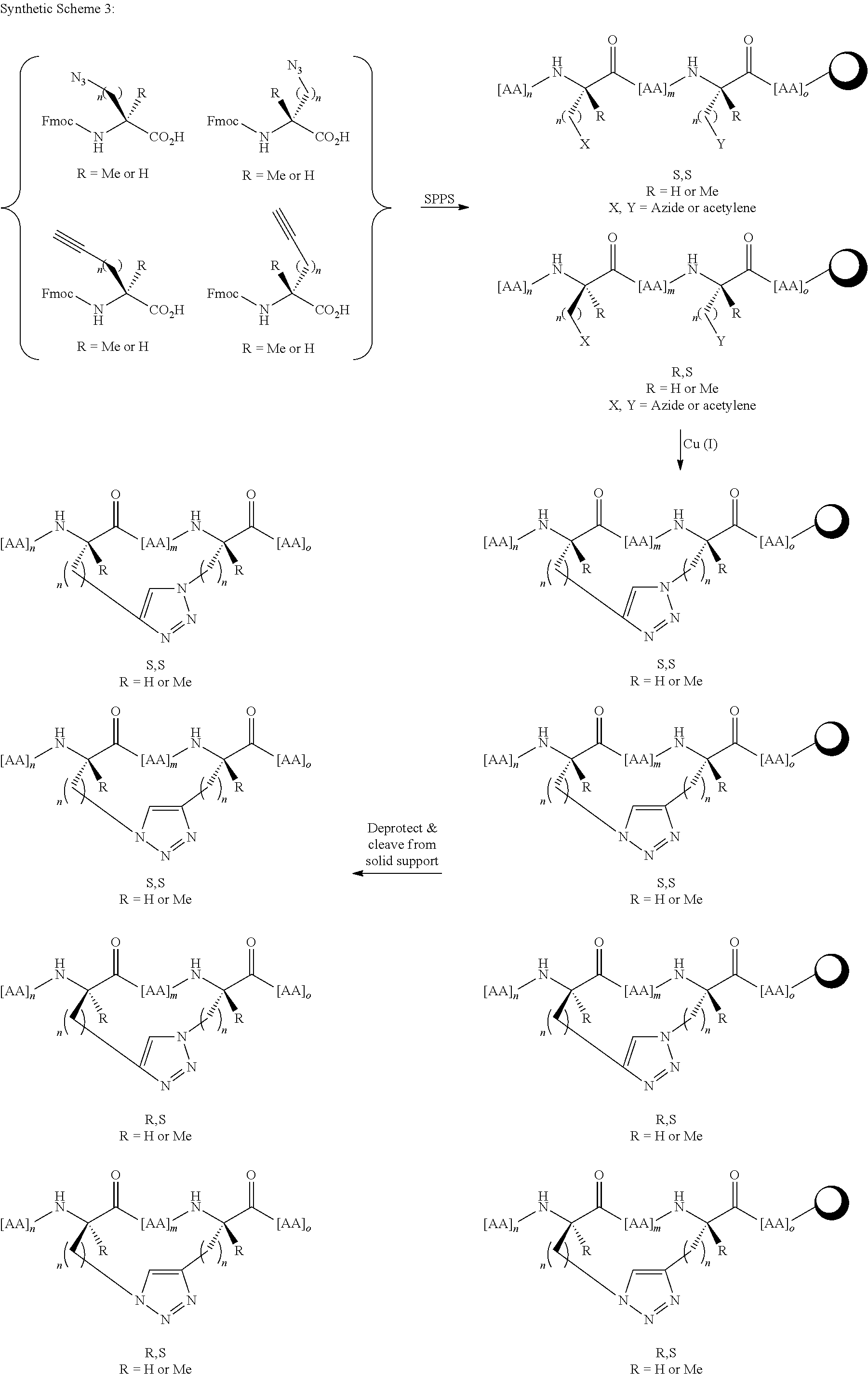

In the general method for the synthesis of peptidomimetic macrocycles shown in Synthetic Scheme 3, the peptidomimetic precursor contains an azide moiety and an alkyne moiety and is synthesized by solid-phase peptide synthesis (SPPS) using the commercially available amino acid N-α-Fmoc-L-propargylglycine and the N-α-Fmoc-protected forms of the amino acids (S)-2-amino-2-methyl-4-pentynoic acid, (S)-2-amino-6-heptynoic acid, (S)-2-amino-2-methyl-6-heptynoic acid, N-methyl-s-azido-L-lysine, and N-methyl-ε-azido-D-lysine. The peptidomimetic precursor is reacted with a macrocyclization reagent such as a Cu(I) reagent on the resin as a crude mixture (Rostovtsev et al. (2002), Angew. Chem. Int. Ed. 41:2596-2599; Tornoe et al. (2002), J. Org. Chem. 67:3057-3064; Deiters et al. (2003), J. Am. Chem. Soc. 125:11782-11783; Punna et al. (2005), Angew. Chem. Int. Ed. 44:2215-2220). The resultant triazole-containing peptidomimetic macrocycle is then deprotected and cleaved from the solid-phase resin by standard conditions (e.g., strong acid such as 95% TFA). In some embodiments, the macrocyclization step is performed in a solvent chosen from the group consisting of CH₂Cl₂, ClCH₂CH₂Cl, DMF, THF, NMP, DIPEA, 2,6-lutidine, pyridine, DMSO, H₂O or a mixture thereof. In some embodiments, a solution of a reducing agent such as sodium ascorbate may be used. In some embodiments, the macrocyclization step is performed in a buffered aqueous or partially aqueous solvent.

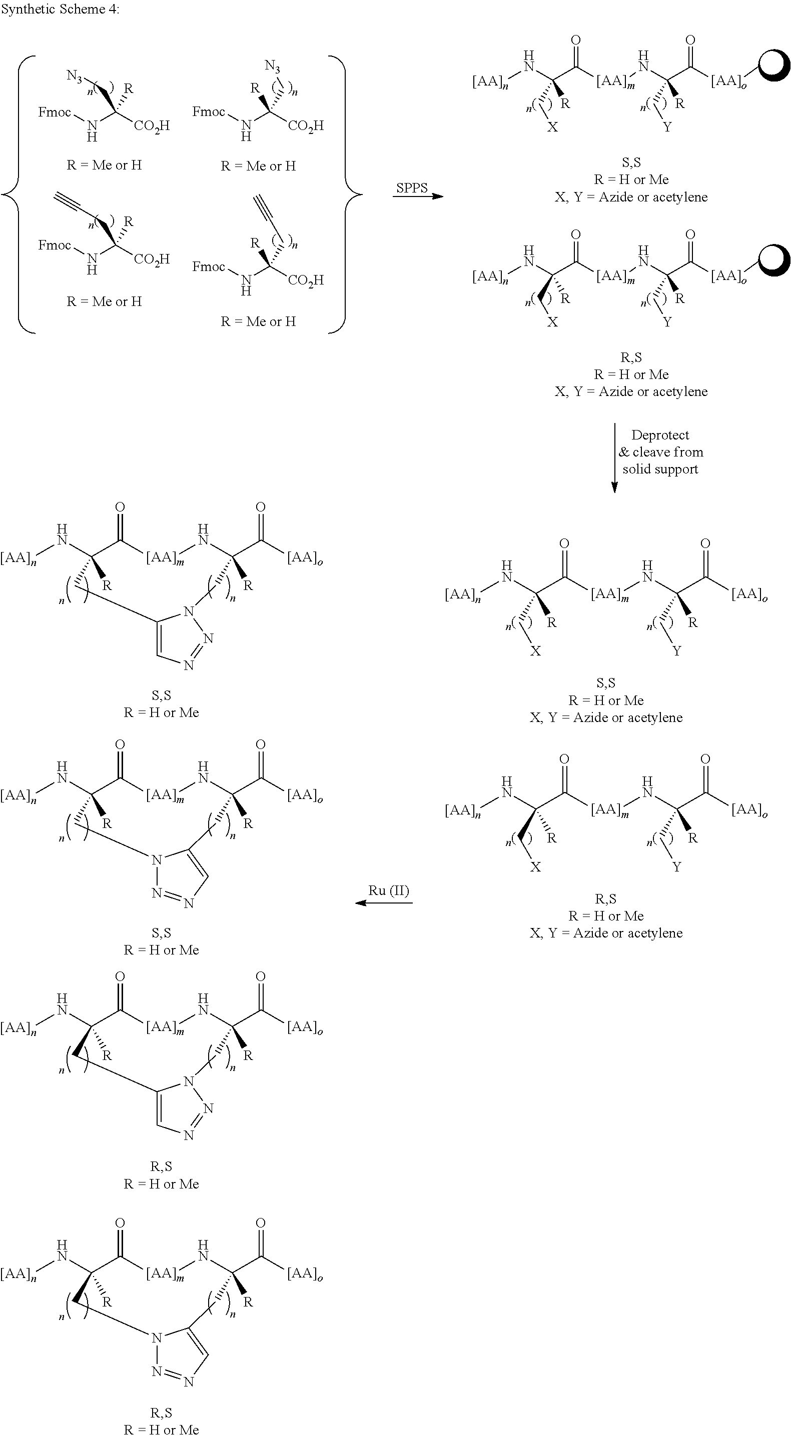

In the general method for the synthesis of peptidomimetic macrocycles shown in Synthetic Scheme 4, the peptidomimetic precursor contains an azide moiety and an alkyne moiety and is synthesized by solution-phase or solid-phase peptide synthesis (SPPS) using the commercially available amino acid N-α-Fmoc-L-propargylglycine and the N-α-Fmoc-protected forms of the amino acids (S)-2-amino-2-methyl-4-pentynoic acid, (S)-2-amino-6-heptynoic acid, (S)-2-amino-2-methyl-6-heptynoic acid, N-methyl-ε-azido-L-lysine, and N-methyl-ε-azido-D-lysine. The peptidomimetic precursor is then deprotected and cleaved from the solid-phase resin by standard conditions (e.g., strong acid such as 95% TFA). The peptidomimetic precursor is reacted as a crude mixture or is purified prior to reaction with a macrocyclization reagent such as a Ru(II) reagents, for example Cp*RuCl(PPh₃)₂ or [Cp*RuCl]₄ (Rasmussen et al. (2007), Org. Lett. 9:5337-5339; Zhang et al. (2005), J. Am. Chem. Soc. 127:15998-15999). In some embodiments, the macrocyclization step is performed in a solvent chosen from the group consisting of DMF, CH₃CN, benzene, toluene and THF.

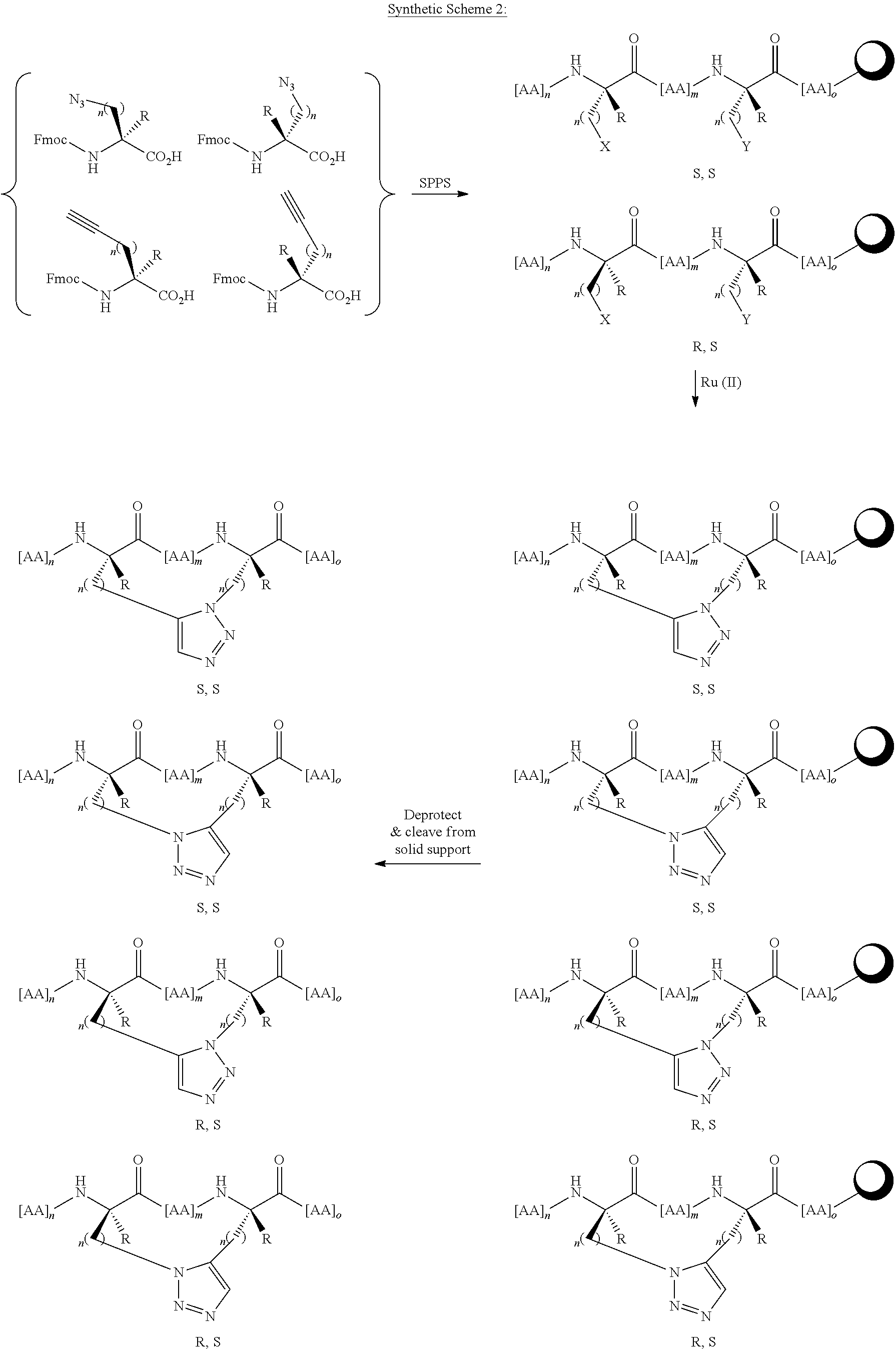

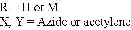

In the general method for the synthesis of peptidomimetic macrocycles shown in Synthetic Scheme 5, the peptidomimetic precursor contains an azide moiety and an alkyne moiety and is synthesized by solid-phase peptide synthesis (SPPS) using the commercially available amino acid N-α-Fmoc-L-propargylglycine and the N-α-Fmoc-protected forms of the amino acids (S)-2-amino-2-methyl-4-pentynoic acid, (S)-2-amino-6-heptynoic acid, (S)-2-amino-2-methyl-6-heptynoic acid, N-methyl-s-azido-L-lysine, N-methyl-ε-azido-D-lysine, 2-amino-7-azido-2-methylheptanoic acid and 2-amino-6-azido-2-methylhexanoic acid. The peptidomimetic precursor is reacted with a macrocyclization reagent such as a Ru(II) reagent on the resin as a crude mixture. For example, the reagent can be Cp*RuCl(PPh₃)₂ or [Cp*RuCl]₄ (Rasmussen et al. (2007), Org. Lett. 9:5337-5339; Zhang et al. (2005), J. Am. Chem. Soc. 127:15998-15999). In some embodiments, the macrocyclization step is performed in a solvent chosen from the group consisting of CH₂Cl₂, ClCH₂CH₂Cl, CH₃CN, DMF, benzene, toluene and THF.

Several exemplary peptidomimetic macrocycles are shown in Table 5 (SEQ ID NOS 91-108, respectively, in order of appearance). “Nle” represents norleucine and replaces a methionine residue. It is envisioned that similar linkers are used to synthesize peptidomimetic macrocycles based on the polypeptide sequences disclosed in Table 1 through Table 4.

TABLE 5

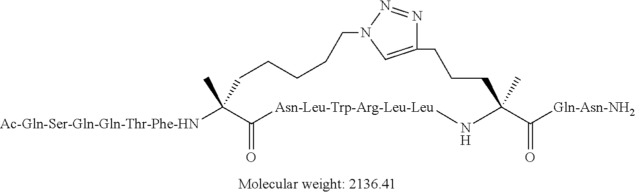

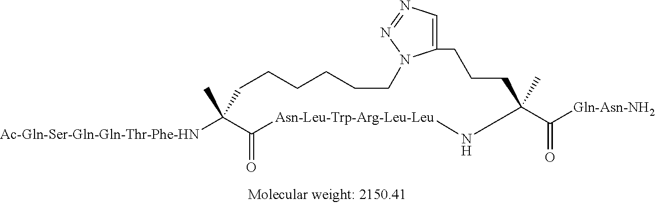

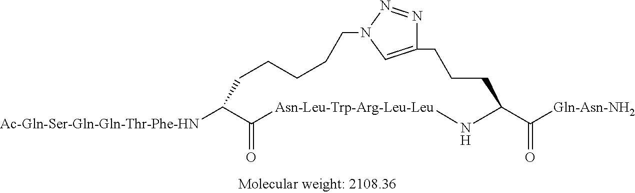

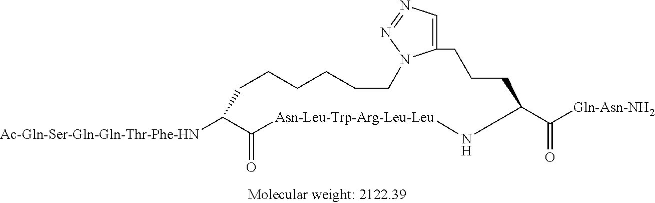

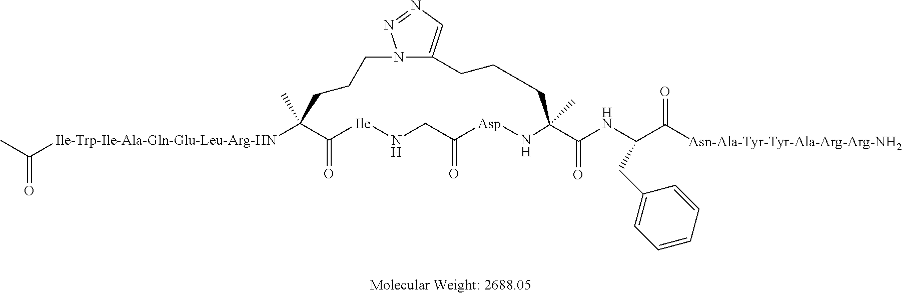

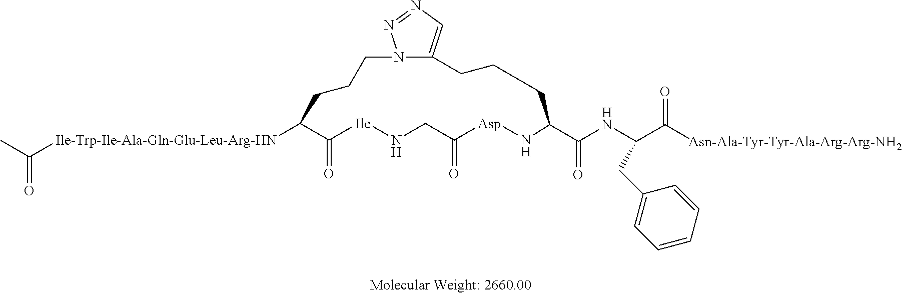

| | |
|---|---|
| 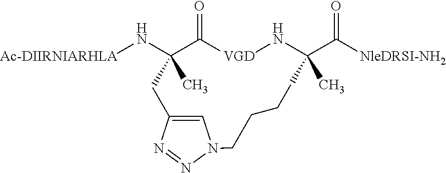 | MW = 2464 |
| 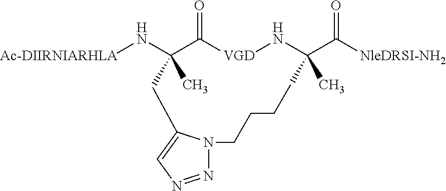 | MW = 2464 |
| 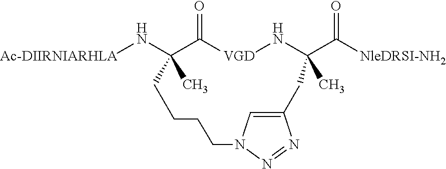 | MW = 2464 |
| 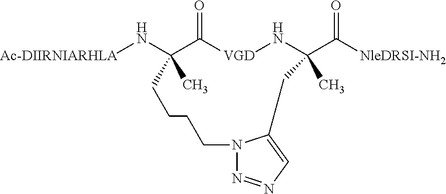 | MW = 2464 |
| 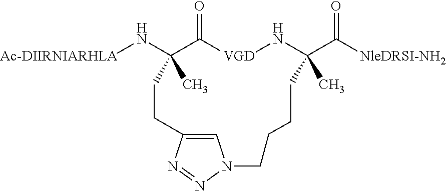 | MW = 2478 |
| 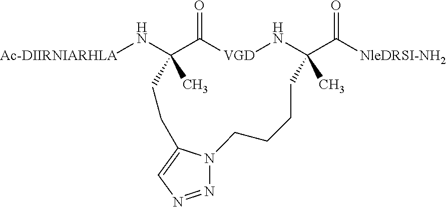 | MW = 2478 |
| 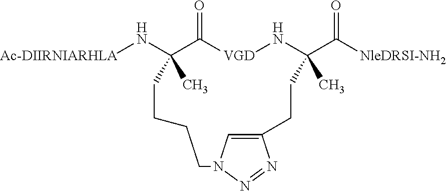 | MW = 2478 |
| 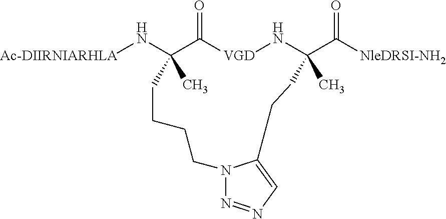 | MW = 2478 |
| 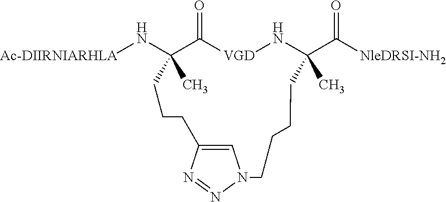 | MW = 2492 |
| 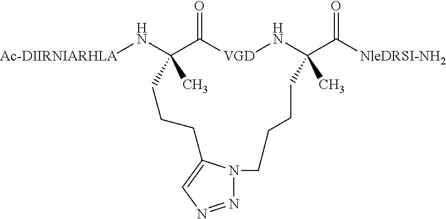 | MW = 2492 |
| 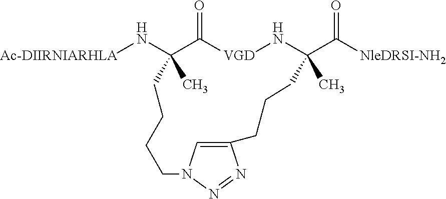 | MW = 2492 |
| 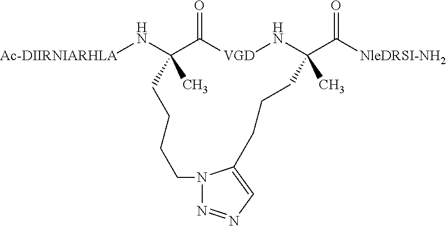 | MW = 2492 |

Table 5 shows exemplary peptidomimetic macrocycles of the invention. “Nle” represents norleucine.

The present invention contemplates the use of non-naturally-occurring amino acids and amino acid analogs in the synthesis of the peptidomimetic macrocycles described herein. Any amino acid or amino acid analog amenable to the synthetic methods employed for the synthesis of stable triazole containing peptidomimetic macrocycles can be used in the present invention. For example, L-propargylglycine is contemplated as a useful amino acid in the present invention. However, other alkyne-containing amino acids that contain a different amino acid side chain are also useful in the invention. For example, L-propargylglycine contains one methylene unit between the α-carbon of the amino acid and the alkyne of the amino acid side chain. The invention also contemplates the use of amino acids with multiple methylene units between the α-carbon and the alkyne. Also, the azido-analogs of amino acids L-lysine, D-lysine, alpha-methyl-L-lysine, and alpha-methyl-D-lysine are contemplated as useful amino acids in the present invention. However, other terminal azide amino acids that contain a different amino acid side chain are also useful in the invention. For example, the azido-analog of L-lysine contains four methylene units between the α-carbon of the amino acid and the terminal azide of the amino acid side chain. The invention also contemplates the use of amino acids with fewer than or greater than four methylene units between the α-carbon and the terminal azide. Table 6 shows some amino acids useful in the preparation of peptidomimetic macrocycles of the invention.

TABLE 6

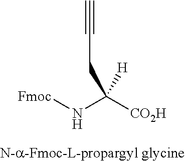

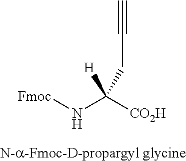

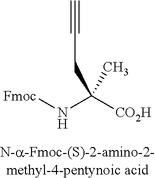

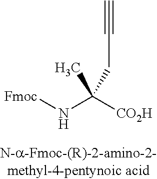

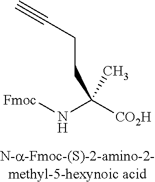

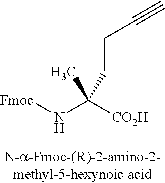

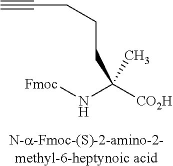

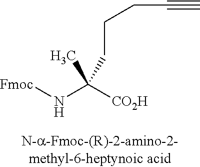

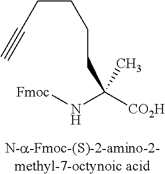

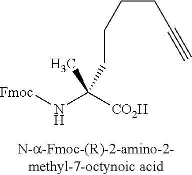

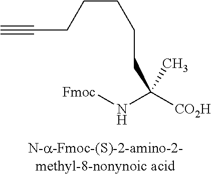

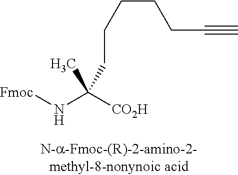

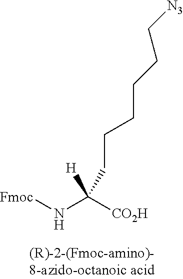

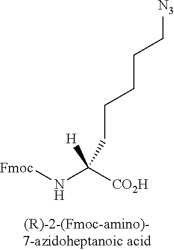

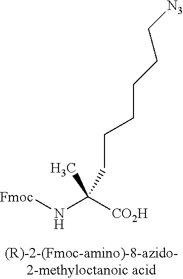

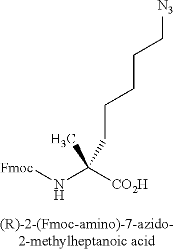

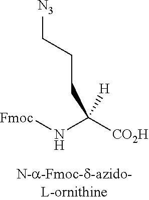

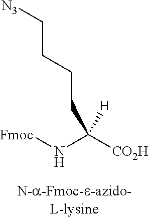

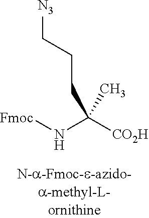

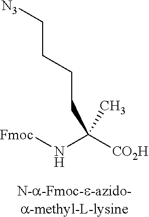

Table 6 shows exemplary amino acids useful in the preparation of peptidomimetic macrocycles of the invention.

In some embodiments the amino acids and amino acid analogs are of the D-configuration. In other embodiments they are of the L-configuration. In some embodiments, some of the amino acids and amino acid analogs contained in the peptidomimetic are of the D-configuration while some of the amino acids and amino acid analogs are of the L-configuration. In some embodiments the amino acid analogs are α,α-disubstituted, such as α-methyl-L-propargylglycine, α-methyl-D-propargylglycine, ε-azido-alpha-methyl-L-lysine, and ε-azido-alpha-methyl-D-lysine. In some embodiments the amino acid analogs are N-alkylated, e.g., N-methyl-L-propargylglycine, N-methyl-D-propargylglycine, N-methyl-ε-azido-L-lysine, and N-methyl-ε-azido-D-lysine.

In some embodiments, the —NH moiety of the amino acid is protected using a protecting group, including without limitation -Fmoc and -Boc. In other embodiments, the amino acid is not protected prior to synthesis of the peptidomimetic macrocycle.

In other embodiments, peptidomimetic macrocycles of Formula III are synthesized. The following synthetic schemes describe the preparation of such compounds. To simplify the drawings, the illustrative schemes depict amino acid analogs derived from L- or D-cysteine, in which L₁ and L₃ are both —(CH₂)—. However, as noted throughout the detailed description above, many other amino acid analogs can be employed in which L₁ and L₃ can be independently selected from the various structures disclosed herein. The symbols “[AA]ₘ”, “[AA]ₙ”, “[AA]ₒ” represent a sequence of amide bond-linked moieties such as natural or unnatural amino acids. As described previously, each occurrence of “AA” is independent of any other occurrence of “AA”, and a formula such as “[AA]ₘ” encompasses, for example, sequences of non-identical amino acids as well as sequences of identical amino acids.

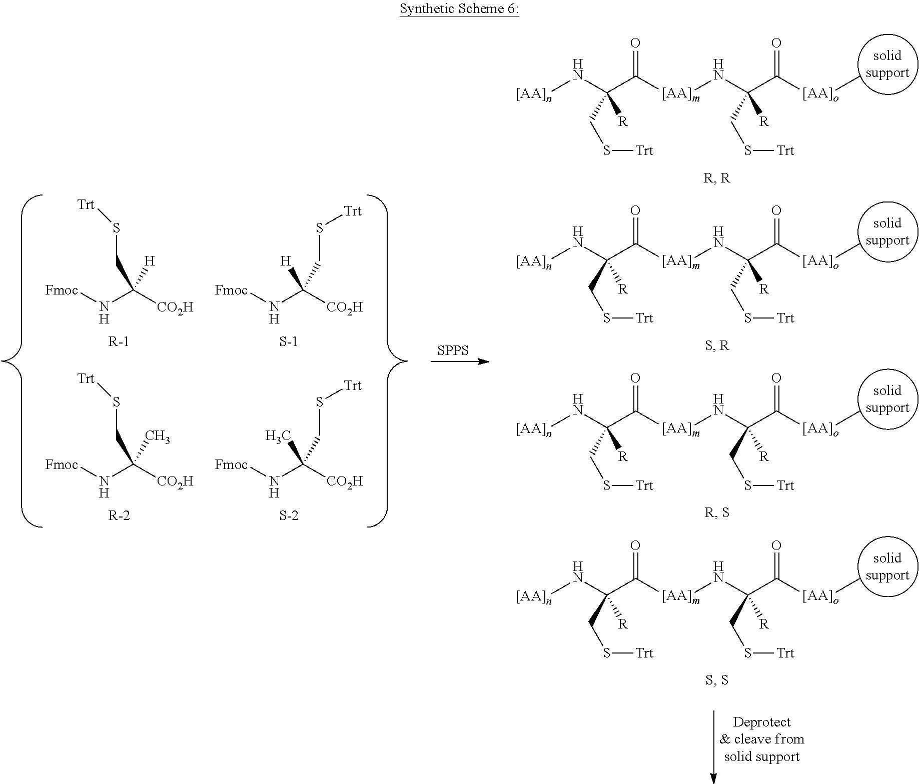

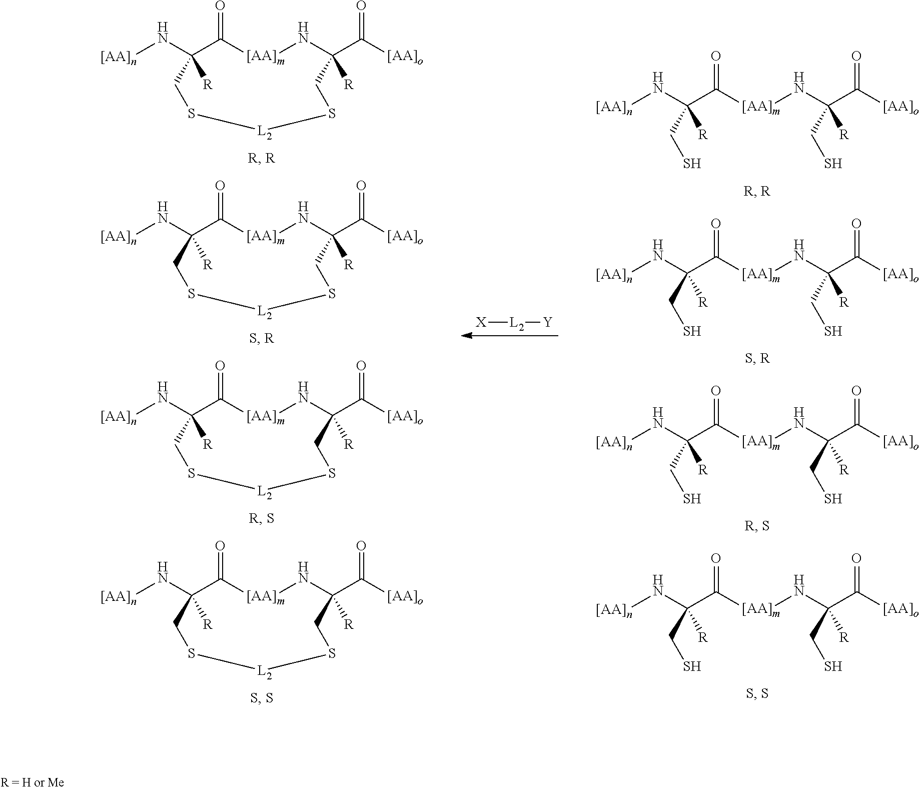

In Scheme 6, the peptidomimetic precursor contains two —SH moieties and is synthesized by solid-phase peptide synthesis (SPPS) using commercially available N-α-Fmoc amino acids such as N-α-Fmoc-S-trityl-L-cysteine or N-α-Fmoc-S-trityl-D-cysteine. Alpha-methylated versions of D-cysteine or L-cysteine are generated by known methods (Seebach et al. (1996), Angew. Chem. Int. Ed. Engl. 35:2708-2748, and references therein) and then converted to the appropriately protected N-α-Fmoc-S-trityl monomers by known methods (“Bioorganic Chemistry: Peptides and Proteins”, Oxford University Press, New York: 1998, the entire contents of which are incorporated herein by reference). The precursor peptidomimetic is then deprotected and cleaved from the solid-phase resin by standard conditions (e.g., strong acid such as 95% TFA). The precursor peptidomimetic is reacted as a crude mixture or is purified prior to reaction with X-L₂-Y in organic or aqueous solutions. In some embodiments the alkylation reaction is performed under dilute conditions (i.e. 0.15 mmol/L) to favor macrocyclization and to avoid polymerization. In some embodiments, the alkylation reaction is performed in organic solutions such as liquid NH₃ (Mosberg et al. (1985), J. Am. Chem. Soc. 107:2986-2987; Szewczuk et al. (1992), Int. J. Peptide Protein Res. 40:233-242), NH₃/MeOH, or NH₃/DMF (Or et al. (1991), J. Org. Chem. 56:3146-3149). In other embodiments, the alkylation is performed in an aqueous solution such as 6M guanidinium HCL, pH 8 (Brunel et al. (2005), Chem. Commun (20):2552-2554). In other embodiments, the solvent used for the alkylation reaction is DMF or dichloroethane.

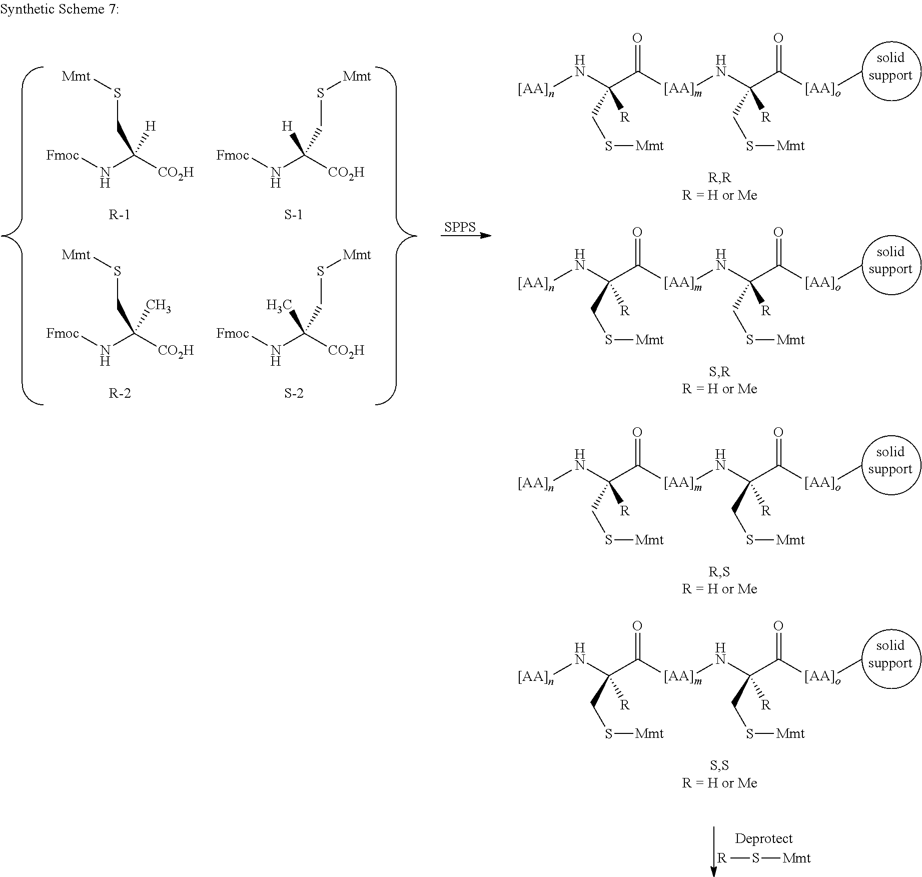

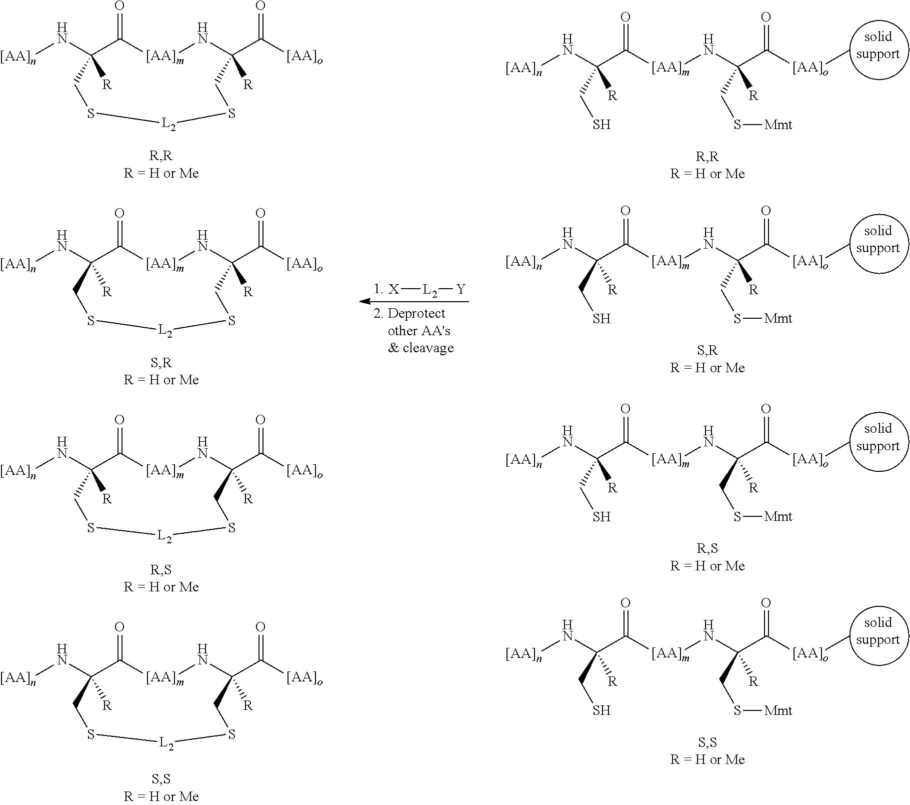

In Scheme 7, the precursor peptidomimetic contains two or more —SH moieties, of which two are specially protected to allow their selective deprotection and subsequent alkylation for macrocycle formation. The precursor peptidomimetic is synthesized by solid-phase peptide synthesis (SPPS) using commercially available N-α-Fmoc amino acids such as N-α-Fmoc-S-p-methoxytrityl-L-cysteine or N-α-Fmoc-S-p-methoxytrityl-D-cysteine. Alpha-methylated versions of D-cysteine or L-cysteine are generated by known methods (Seebach et al. (1996), Angew. Chem. Int. Ed. Engl. 35:2708-2748, and references therein) and then converted to the appropriately protected N-α-Fmoc-S-p-methoxytrityl monomers by known methods (Bioorganic Chemistry: Peptides and Proteins, Oxford University Press, New York: 1998, the entire contents of which are incorporated herein by reference). The Mmt protecting groups of the peptidomimetic precursor are then selectively cleaved by standard conditions (e.g., mild acid such as 1% TFA in DCM). The precursor peptidomimetic is then reacted on the resin with X-L₂-Y in an organic solution. For example, the reaction takes place in the presence of a hindered base such as diisopropylethylamine. In some embodiments, the alkylation reaction is performed in organic solutions such as liquid NH₃ (Mosberg et al. (1985), J. Am. Chem. Soc. 107:2986-2987; Szewczuk et al. (1992), Int. J. Peptide Protein Res. 40:233-242), NH₃/MeOH or NH₃/DMF (Or et al. (1991), J. Org. Chem. 56:3146-3149). In other embodiments, the alkylation reaction is performed in DMF or dichloroethane. The peptidomimetic macrocycle is then deprotected and cleaved from the solid-phase resin by standard conditions (e.g., strong acid such as 95% TFA).

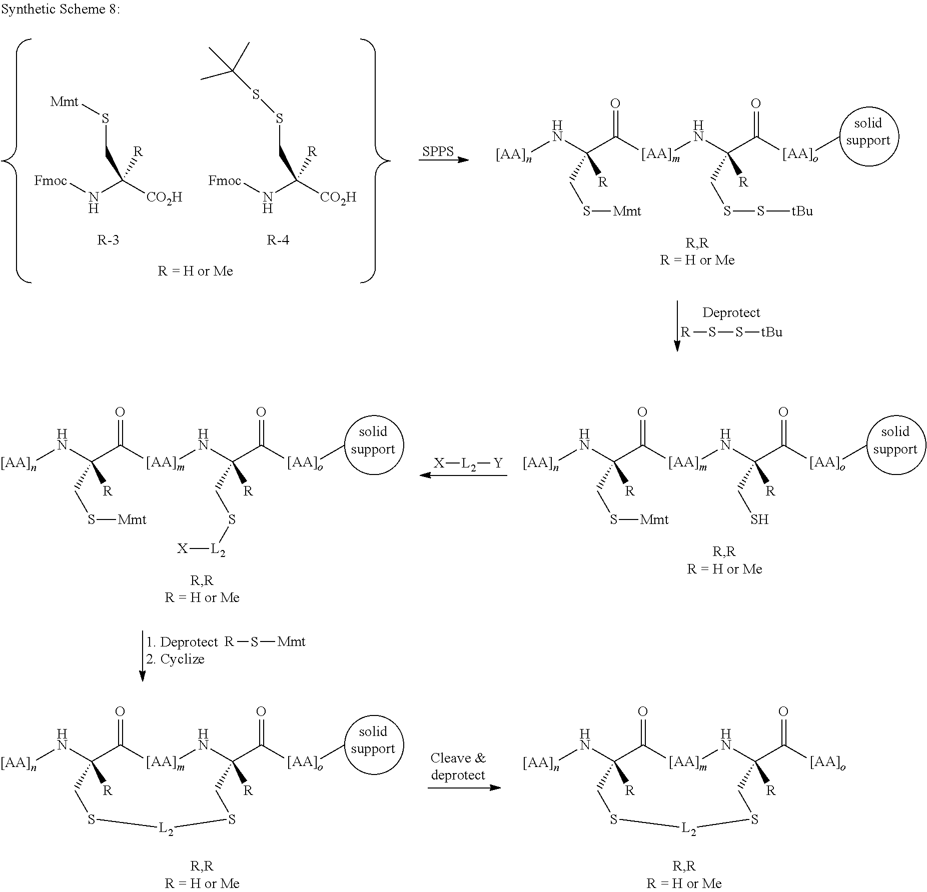

In Scheme 8, the peptidomimetic precursor contains two or more —SH moieties, of which two are specially protected to allow their selective deprotection and subsequent alkylation for macrocycle formation. The peptidomimetic precursor is synthesized by solid-phase peptide synthesis (SPPS) using commercially available N-α-Fmoc amino acids such as N-α-Fmoc-S-p-methoxytrityl-L-cysteine, N-α-Fmoc-S-p-methoxytrityl-D-cysteine, N-α-Fmoc-S—S-t-butyl-L-cysteine, and N-α-Fmoc-S—S-t-butyl-D-cysteine. Alpha-methylated versions of D-cysteine or L-cysteine are generated by known methods (Seebach et al. (1996), Angew. Chem. Int. Ed. Engl. 35:2708-2748, and references therein) and then converted to the appropriately protected N-α-Fmoc-S-p-methoxytrityl or N-α-Fmoc-S—S-t-butyl monomers by known methods (Bioorganic Chemistry: Peptides and Proteins, Oxford University Press, New York: 1998, the entire contents of which are incorporated herein by reference). The S—S-tButyl protecting group of the peptidomimetic precursor is selectively cleaved by known conditions (e.g., 20% 2-mercaptoethanol in DMF, reference: Galande et al. (2005), J. Comb. Chem. 7:174-177). The precursor peptidomimetic is then reacted on the resin with a molar excess of X-L₂-Y in an organic solution. For example, the reaction takes place in the presence of a hindered base such as diisopropylethylamine. The Mmt protecting group of the peptidomimetic precursor is then selectively cleaved by standard conditions (e.g., mild acid such as 1% TFA in DCM). The peptidomimetic precursor is then cyclized on the resin by treatment with a hindered base in organic solutions. In some embodiments, the alkylation reaction is performed in organic solutions such as NH₃/MeOH or NH₃/DMF (Or et al. (1991), J. Org. Chem. 56:3146-3149). The peptidomimetic macrocycle is then deprotected and cleaved from the solid-phase resin by standard conditions (e.g., strong acid such as 95% TFA).

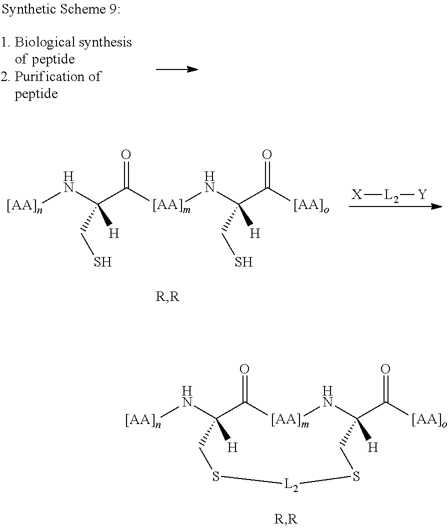

In Scheme 9, the peptidomimetic precursor contains two L-cysteine moieties. The peptidomimetic precursor is synthesized by known biological expression systems in living cells or by known in vitro, cell-free, expression methods. The precursor peptidomimetic is reacted as a crude mixture or is purified prior to reaction with X-L2-Y in organic or aqueous solutions. In some embodiments the alkylation reaction is performed under dilute conditions (i.e. 0.15 mmol/L) to favor macrocyclization and to avoid polymerization. In some embodiments, the alkylation reaction is performed in organic solutions such as liquid NH₃ (Mosberg et al. (1985), J. Am. Chem. Soc. 107:2986-2987; Szewczuk et al. (1992), Int. J. Peptide Protein Res. 40:233-242), NH₃/MeOH, or NH₃/DMF (Or et al. (1991), J. Org. Chem. 56:3146-3149). In other embodiments, the alkylation is performed in an aqueous solution such as 6M guanidinium HCL, pH 8 (Brunel et al. (2005), Chem. Commun (20):2552-2554). In other embodiments, the alkylation is performed in DMF or dichloroethane. In another embodiment, the alkylation is performed in non-denaturing aqueous solutions, and in yet another embodiment the alkylation is performed under conditions that favor α-helical structure formation. In yet another embodiment, the alkylation is performed under conditions that favor the binding of the precursor peptidomimetic to another protein, so as to induce the formation of the bound α-helical conformation during the alkylation.

Various embodiments for X and Y are envisioned which are suitable for reacting with thiol groups. In general, each X or Y is independently be selected from the general category shown in Table 5. For example, X and Y are halides such as —Cl, —Br or —I. Any of the macrocycle-forming linkers described herein may be used in any combination with any of the sequences shown in Tables 1-4 and also with any of the R- substituents indicated herein.

TABLE 7
Examples of Reactive Groups Capable of
Reacting with Thiol Groups and Resulting Linkages

| | Resulting Covalent |
|---|---|
| X or Y | Linkage |
| acrylamide | Thioether |
| halide (e.g. alkyl or aryl halide) | Thioether |
| sulfonate | Thioether |
| aziridine | Thioether |
| epoxide | Thioether |
| haloacetamide | Thioether |
| maleimide | Thioether |
| sulfonate ester | Thioether |

Table 8 shows exemplary macrocycles of the invention (SEQ ID NOS 109-114, respectively, in order of appearance). “N_{L}” represents norleucine and replaces a methionine residue. It is envisioned that similar linkers are used to synthesize peptidomimetic macrocycles based on the polypeptide sequences disclosed in Table 1 through Table 4.

TABLE 8
Examples of Peptidomimetic Macrocycles of the Invention

| | |
|---|---|
| 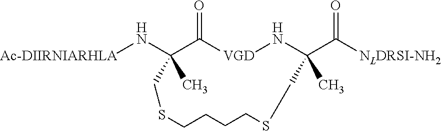 | MW = 2477 |
| 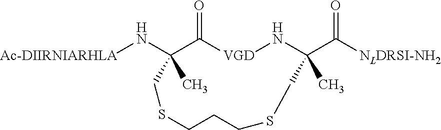 | MW = 2463 |
| 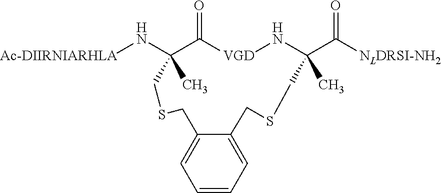 | MW = 2525 |
| 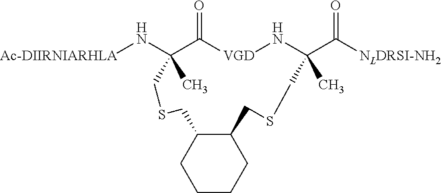 | MW = 2531 |
| 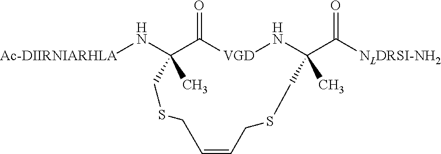 | MW = 2475 |
| 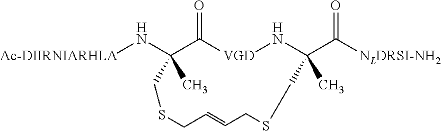 | MW = 2475 |

For the examples shown in this table, “N_{L}” represents norleucine.

The present invention contemplates the use of both naturally-occurring and non-naturally-occurring amino acids and amino acid analogs in the synthesis of the peptidomimetic macrocycles of Formula (III). Any amino acid or amino acid analog amenable to the synthetic methods employed for the synthesis of stable bis-sulfhydryl containing peptidomimetic macrocycles can be used in the present invention. For example, cysteine is contemplated as a useful amino acid in the present invention. However, sulfur containing amino acids other than cysteine that contain a different amino acid side chain are also useful. For example, cysteine contains one methylene unit between the α-carbon of the amino acid and the terminal —SH of the amino acid side chain. The invention also contemplates the use of amino acids with multiple methylene units between the α-carbon and the terminal —SH. Non-limiting examples include α-methyl-L-homocysteine and α-methyl-D-homocysteine. In some embodiments the amino acids and amino acid analogs are of the D- configuration. In other embodiments they are of the L- configuration. In some embodiments, some of the amino acids and amino acid analogs contained in the peptidomimetic are of the D- configuration while some of the amino acids and amino acid analogs are of the L- configuration. In some embodiments the amino acid analogs are α,α-disubstituted, such as α-methyl-L-cysteine and α-methyl-D-cysteine.

The invention includes macrocycles in which macrocycle-forming linkers are used to link two or more —SH moieties in the peptidomimetic precursors to form the peptidomimetic macrocycles of the invention. As described above, the macrocycle-forming linkers impart conformational rigidity, increased metabolic stability and/or increased cell penetrability. Furthermore, in some embodiments, the macrocycle-forming linkages stabilize the α-helical secondary structure of the peptidomimetic macrocyles. The macrocycle-forming linkers are of the formula X-L₂-Y, wherein both X and Y are the same or different moieties, as defined above. Both X and Y have the chemical characteristics that allow one macrocycle-forming linker -L₂- to bis alkylate the bis-sulfhydryl containing peptidomimetic precursor. As defined above, the linker -L₂- includes alkylene, alkenylene, alkynylene, heteroalkylene, cycloalkylene, heterocycloalkylene, cycloarylene, or heterocycloarylene, or —R₄—K—R₄—, all of which can be optionally substituted with an R₅ group, as defined above. Furthermore, one to three carbon atoms within the macrocycle-forming linkers -L₂-, other than the carbons attached to the —SH of the sulfhydryl containing amino acid, are optionally substituted with a heteroatom such as N, S or O.

The L₂ component of the macrocycle-forming linker X-L₂-Y may be varied in length depending on, among other things, the distance between the positions of the two amino acid analogs used to form the peptidomimetic macrocycle. Furthermore, as the lengths of L₁ and/or L₃ components of the macrocycle-forming linker are varied, the length of L₂ can also be varied in order to create a linker of appropriate overall length for forming a stable peptidomimetic macrocycle. For example, if the amino acid analogs used are varied by adding an additional methylene unit to each of L₁ and L₃, the length of L₂ are decreased in length by the equivalent of approximately two methylene units to compensate for the increased lengths of L₁ and L₃.

In some embodiments, L₂ is an alkylene group of the formula —(CH₂)ₙ—, where n is an integer between about 1 and about 15. For example, n is 1, 2, 3, 4, 5, 6, 7, 8, 9 or 10. In other embodiments, L₂ is an alkenylene group. In still other embodiments, L₂ is an aryl group.

Table 9 shows additional embodiments of X-L₂-Y groups.

TABLE 9
Exemplary X—L₂—Y groups of the invention.

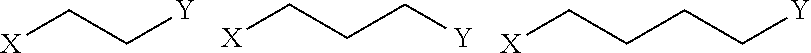

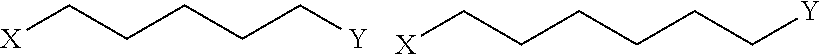

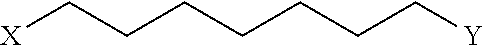

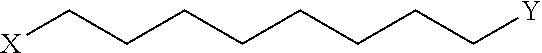

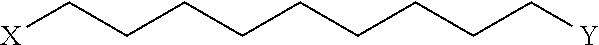

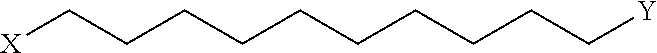

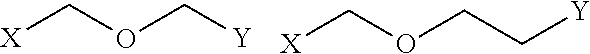

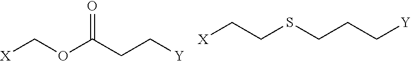

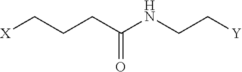

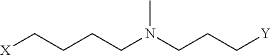

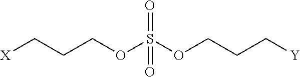

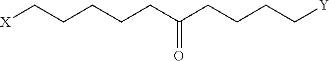

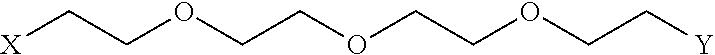

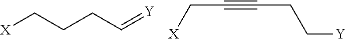

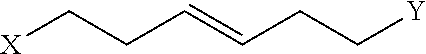

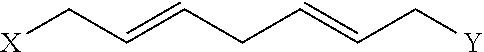

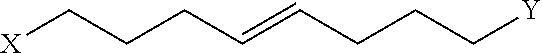

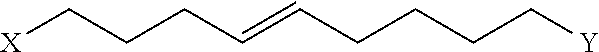

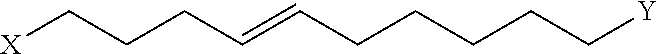

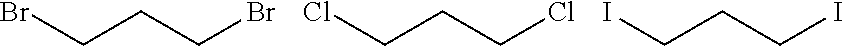

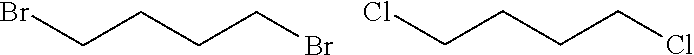

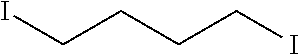

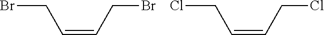

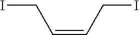

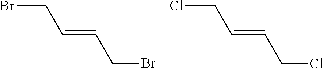 [SMILES: BrC/C=C/CBr ; ClC/C=C/CCl]

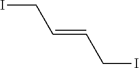

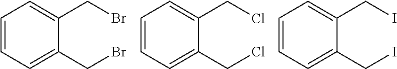

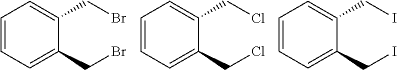

Each X and Y in this table, is, for example, independently Cl—, Br— or I—.

Additional methods of forming peptidomimetic macrocycles which are envisioned as suitable to perform the present invention include those disclosed by Mustapa, M. Firouz Mohd et al., J. Org. Chem (2003), 68, pp. 8193-8198; Yang, Bin et al. Bioorg Med. Chem. Lett. (2004), 14, pp. 1403-1406; U.S. Pat. No. 5,364,851; U.S. Pat. No. 5,446,128; U.S. Pat. No. 5,824,483; U.S. Pat. No. 6,713,280; and U.S. Pat. No. 7,202,332. In such embodiments, aminoacid precursors are used containing an additional substituent R- at the alpha position. Such aminoacids are incorporated into the macrocycle precursor at the desired positions, which may be at the positions where the crosslinker is substituted or, alternatively, elsewhere in the sequence of the macrocycle precursor. Cyclization of the precursor is then effected according to the indicated method.

### Assays

The properties of the peptidomimetic macrocycles of the invention are assayed, for example, by using the methods described below. In some embodiments, a peptidomimetic macrocycle of the invention has improved biological properties relative to a corresponding polypeptide lacking the substituents described herein.

### Assay to Determine α-Helicity.

In solution, the secondary structure of polypeptides with α-helical domains will reach a dynamic equilibrium between random coil structures and α-helical structures, often expressed as a “percent helicity”. Thus, for example, unmodified pro-apoptotic BH3 domains are predominantly random coils in solution, with α-helical content usually under 25%. Peptidomimetic macrocycles with optimized linkers, on the other hand, possess, for example, an alpha-helicity that is at least two-fold greater than that of a corresponding macrocycle lacking the R- substituent. In some embodiments, macrocycles of the invention will possess an alpha-helicity of greater than 50%. To assay the helicity of peptidomimetic macrocyles of the invention, such as BH3 domain-based macrocycles, the compounds are dissolved in an aqueous solution (e.g. 50 mM potassium phosphate solution at pH 7, or distilled H₂O, to concentrations of 25-50 μM). Circular dichroism (CD) spectra are obtained on a spectropolarimeter (e.g., Jasco J-710) using standard measurement parameters (e.g. temperature, 20° C.; wavelength, 190-260 nm; step resolution, 0.5 nm; speed, 20 nm/sec; accumulations, 10; response, 1 sec; bandwidth, 1 nm; path length, 0.1 cm). The α-helical content of each peptide is calculated by dividing the mean residue ellipticity (e.g. [Φ]222obs) by the reported value for a model helical decapeptide (Yang et al. (1986), Methods Enzymol. 130:208)).

Assay to Determine Melting Temperature (Tm).

A peptidomimetic macrocycle of the invention comprising a secondary structure such as an α-helix exhibits, for example, a higher melting temperature than a corresponding macrocycle lacking the R- substituent. Typically peptidomimetic macrocycles of the invention exhibit Tm of >60° C. representing a highly stable structure in aqueous solutions. To assay the effect of macrocycle formation on melting temperature, peptidomimetic macrocycles or unmodified peptides are dissolved in distilled H₂O (e.g. at a final concentration of 50 μM) and the Tm is determined by measuring the change in ellipticity over a temperature range (e.g. 4 to 95° C.) on a spectropolarimeter (e.g., Jasco J-710) using standard parameters (e.g. wavelength 222 nm; step resolution, 0.5 nm; speed, 20 nm/sec; accumulations, 10; response, 1 sec; bandwidth, 1 nm; temperature increase rate: 1° C./min; path length, 0.1 cm).

### Protease Resistance Assay.

The amide bond of the peptide backbone is susceptible to hydrolysis by proteases, thereby rendering peptidic compounds vulnerable to rapid degradation in vivo. Peptide helix formation, however, typically buries the amide backbone and therefore may shield it from proteolytic cleavage. The peptidomimetic macrocycles of the present invention may be subjected to in vitro pepsin and trypsin proteolysis to assess for any change in degradation rate compared to a corresponding uncrosslinked) polypeptide. For example, the peptidomimetic macrocycle and a corresponding (unsubstituted) polypeptide are incubated with peptidases, pepsin or trypsin immobilized on silica gel and the reactions quenched at various time points by addition of 2% trifluoracetic acid in acetonitrile/1,1,1,3,3,3-hexafluoro-2-propanol. Subsequent HPLC injection is made for mass spectrometry-based quantification of the residual substrate in the multiple-reaction monitoring mode (MRM) of chromatographic peak detection. Briefly, the peptidomimetic macrocycle and peptidomimetic precursor (5 μM) are incubated with pepsin or trypsin silica gel (Princeton Separations) (S/E ˜50) for 0, 10, 20, 30, and 60 minutes. Reactions are quenched by addition of 2% trifluoracetic acid in acetonitrile/1,1,1,3,3,3-hexafluoro-2-propanol, and remaining substrate in the isolated supernatant is quantified by MRM peak detection. The proteolytic reaction displays first order kinetics and the rate constant, k, is determined from a plot of ln [S] versus time (k=−1×slope). The reaction half-life is calculated using the formula T1/2=ln 2/k.

### Ex Vivo Stability Assay.

Peptidomimetic macrocycles with optimized linkers possess, for example, an ex vivo half-life that is at least two-fold greater than that of a corresponding macrocycle lacking the R- substituent, and possess an ex vivo half-life of 12 hours or more. For ex vivo serum stability studies, a variety of assays may be used. For example, a peptidomimetic macrocycle and a corresponding macrocycle lacking the R- substituent (2 mcg) are incubated with fresh mouse, rat and/or human serum (2 mL) at 37° C. for 0, 1, 2, 4, 8, and 24 hours. Samples of differing macrocycle concentration may be prepared by serial dilution with serum. To determine the level of intact compound, the following procedure may be used: The samples are extracted by transferring 100 μl of sera to 2 ml centrifuge tubes followed by the addition of 10 μL of 50% formic acid and 500 μL acetonitrile and centrifugation at 14,000 RPM for 10 min at 4±2° C. The supernatants are then transferred to fresh 2 ml tubes and evaporated on Turbovap under N₂<10 psi, 37° C. The samples are reconstituted in 100 μL of 50:50 acetonitrile:water and submitted to LC-MS/MS analysis. Equivalent or similar procedures for testing ex vivo stability are known and may be used to determine stability of macrocycles in serum.

### In Vitro Binding Assays.

To assess the binding and affinity of peptidomimetic macrocycles and peptidomimetic precursors to acceptor proteins, a fluorescence polarization assay (FPA) may be used, for example. The FPA technique measures the molecular orientation and mobility using polarized light and fluorescent tracer. When excited with polarized light, fluorescent tracers (e.g., FITC) attached to molecules with high apparent molecular weights (e.g. FITC-labeled peptides bound to a large protein) emit higher levels of polarized fluorescence due to their slower rates of rotation as compared to fluorescent tracers attached to smaller molecules (e.g. FITC-labeled peptides that are free in solution).

For example, fluoresceinated peptidomimetic macrocycles (25 nM) are incubated with the acceptor protein (25-1000 nM) in binding buffer (140 mM NaCl, 50 mM Tris-HCL, pH 7.4) for 30 minutes at room temperature. Binding activity is measured, for example, by fluorescence polarization on a luminescence spectrophotometer (e.g. Perkin-Elmer LS50B). Kd values may be determined by nonlinear regression analysis using, for example, Graphpad Prism software (GraphPad Software, Inc., San Diego, Calif.). A peptidomimetic macrocycle of the invention shows, in some instances, similar or lower Kd than a corresponding macrocycle lacking the R- substituent.

Acceptor proteins for BH3-peptides such as BCL-2, BCL-X_{L}, BAX or MCL1 may, for example, be used in this assay. Acceptor proteins for p53 peptides such as MDM2 or MDMX may also be used in this assay.

### In Vitro Displacement Assays to Characterize Antagonists of Peptide-Protein Interactions.

To assess the binding and affinity of compounds that antagonize the interaction between a peptide (e.g. a BH3 peptide or a p53 peptide) and an acceptor protein, a fluorescence polarization assay (FPA) utilizing a fluoresceinated peptidomimetic macrocycle derived from a peptidomimetic precursor sequence is used, for example. The FPA technique measures the molecular orientation and mobility using polarized light and fluorescent tracer. When excited with polarized light, fluorescent tracers (e.g., FITC) attached to molecules with high apparent molecular weights (e.g. FITC-labeled peptides bound to a large protein) emit higher levels of polarized fluorescence due to their slower rates of rotation as compared to fluorescent tracers attached to smaller molecules (e.g. FITC-labeled peptides that are free in solution). A compound that antagonizes the interaction between the fluoresceinated peptidomimetic macrocycle and an acceptor protein will be detected in a competitive binding FPA experiment.

For example, putative antagonist compounds (1 nM to 1 mM) and a fluoresceinated peptidomimetic macrocycle (25 nM) are incubated with the acceptor protein (50 nM) in binding buffer (140 mM NaCl, 50 mM Tris-HCL, pH 7.4) for 30 minutes at room temperature. Antagonist binding activity is measured, for example, by fluorescence polarization on a luminescence spectrophotometer (e.g. Perkin-Elmer LS50B). Kd values may be determined by nonlinear regression analysis using, for example, Graphpad Prism software (GraphPad Software, Inc., San Diego, Calif.).

Any class of molecule, such as small organic molecules, peptides, oligonucleotides or proteins can be examined as putative antagonists in this assay. Acceptor proteins for BH3-peptides such as BCL2, BCL-XL, BAX or MCL1 can be used in this assay. Additional methods to perform such assays are described in the Example section below.

### Binding Assays in Cell Lysates or Intact Cells.

It is possible to measure binding of peptides or peptidomimetic macrocycles to their natural acceptors in cell lysates or intact cells by immunoprecipitation and pull-down experiments. For example, intact cells are incubated with fluoresceinated (FITC-labeled) or biotinylated compounds for 4 hrs in the absence of serum, followed by serum replacement and further incubation that ranges from 4-18 hrs. Alternatively, cells can be incubated for the duration of the experiment in Opti-MEM (Invitrogen). Cells are then pelleted and incubated in lysis buffer (50 mM Tris [pH 7.6], 150 mM NaCl, 1% CHAPS and protease inhibitor cocktail) for 10 minutes at 4° C. 1% NP-40 or Triton X-100 may be used instead of CHAPS. Extracts are centrifuged at 14,000 rpm for 15 minutes and supernatants collected and incubated with 10 μl goat anti-FITC antibody or streptavidin-coated beads for 2 hrs, rotating at 4° C. followed by further 2 hrs incubation at 4° C. with protein A/G Sepharose (50 μl of 50% bead slurry).). No secondary step is necessary if using streptavidin beads to pull down biotinylated compounds. Alternatively FITC-labeled or biotinylated compounds are incubated with cell lysates, prepared as described above, for 2 hrs, rotating at 4° C. followed by incubation with 10 μl goat anti-FITC antibody or streptavidin-coated beads for 2 hrs, rotating at 4° C. followed by further 2 hrs incubation at 4° C. with protein A/G Sepharose (50 μl of 50% bead slurry), no secondary step is necessary if using streptavidin beads to pull down biotinylated compounds. After quick centrifugation, the pellets may be washed in lysis buffer containing increasing salt concentration (e.g., 150, 300, 500 mM of NaCl). The beads may be then re-equilibrated at 150 mM NaCl before addition of SDS-containing sample buffer and boiling. The beads and cell lysates may be electrophoresed using 4%-12% gradient Bis-Tris gels followed by transfer into Immobilon-P membranes. After blocking, blots may be incubated with an antibody that detects FITC or biotin, respectively and also with one or more antibodies that detect proteins that bind to the peptidomimetic macrocycle, including BCL2, MCL1, BCL-XL, Al, BAX, and BAK. The lysate blots are also probed with anti-Hsc-70 for loading control. Alternatively, after electrophoresis the gel may be silver stained to detect proteins that come down specifically with FITC-labeled or biotinylated compounds.

### Cellular Penetrability Assays.

A peptidomimetic macrocycle is, for example, more cell permeable compared to a corresponding macrocycle lacking the R- substituent. In some embodiments, the peptidomimetic macrocycles are more cell permeable than a corresponding macrocycle lacking the R- substituents. Peptidomimetic macrocycles with optimized linkers possess, for example, cell penetrability that is at least two-fold greater than a corresponding macrocycle lacking the R- substituent, and often 20% or more of the applied peptidomimetic macrocycle will be observed to have penetrated the cell after 4 hours. To measure the cell penetrability of peptidomimetic macrocycles and corresponding macrocycle lacking the R- substituents, intact cells are incubated with fluoresceinated peptidomimetic macrocycles or corresponding uncrosslinked polypeptides (10 μM) for 4 hrs in serum free media at 37° C., washed twice with media and incubated with trypsin (0.25%) for 10 min at 37° C. The cells are washed again and resuspended in PBS. Cellular fluorescence is analyzed, for example, by using either a FACSCalibur flow cytometer or Cellomics' KineticScan® HCS Reader. Additional methods of quantitating cellular penetration may be used. A particular method is described in more detail in the Examples provided.

### Cellular Efficacy Assays.

The efficacy of certain peptidomimetic macrocycles is determined, for example, in cell-based killing assays using a variety of tumorigenic and non-tumorigenic cell lines and primary cells derived from human or mouse cell populations. Cell viability is monitored, for example, over 24-96 hrs of incubation with peptidomimetic macrocycles (0.5 to 50 μM) to identify those that kill at EC₅₀<10 μM. In this context, EC₅₀ refers to the half maximal effective concentration, which is the concentration of peptidomimetic macrocycle at which 50% the population is viable. Several standard assays that measure cell viability are commercially available and are optionally used to assess the efficacy of the peptidomimetic macrocycles. In addition, assays that measure Annexin V and caspase activation are optionally used to assess whether the peptidomimetic macrocycles kill cells by activating the apoptotic machinery. For example, the Cell Titer-glo assay is used which determines cell viability as a function of intracellular ATP concentration.

### In Vivo Stability Assay.

To investigate the in vivo stability of the peptidomimetic macrocycles, the compounds are, for example, administered to mice and/or rats by IV, IP, SC, PO or inhalation routes at concentrations ranging from 0.1 to 50 mg/kg and blood specimens withdrawn at 0′, 5′, 15′, 30′, 1 hr, 4 hrs, 8 hrs, 12 hrs, 24 hrs and 48 hrs post-injection. Levels of intact compound in 25 μL of fresh serum are then measured by LC-MS/MS as described herein.

In vivo Efficacy in Animal Models.

To determine the anti-oncogenic activity of peptidomimetic macrocycles of the invention in vivo, the compounds are, for example, given alone (IP, IV, SC, PO, by inhalation or nasal routes) or in combination with sub-optimal doses of relevant chemotherapy (e.g., cyclophosphamide, doxorubicin, etoposide). In one example, 5×10⁶ SEMK2 cells (established from the bone marrow of a patient with acute lymphoblastic leukemia) that stably express luciferase are injected by tail vein in NOD-SCID, SCID-beige or NOD.IL2rg KO mice 3 hrs after they have been subjected to total body irradiation. Non-radiated mice may also be used for these studies. If left untreated, this form of leukemia is fatal in 3 weeks in this model. The leukemia is readily monitored, for example, by injecting the mice with D-luciferin (60 mg/kg) and imaging the anesthetized animals (e.g., Xenogen In Vivo Imaging System, Caliper Life Sciences, Hopkinton, Mass.). Total body bioluminescence is quantified by integration of photonic flux (photons/sec) by Living Image Software (Caliper Life Sciences, Hopkinton, Mass.). Peptidomimetic macrocycles alone or in combination with sub-optimal doses of relevant chemotherapeutics agents are, for example, administered to leukemic mice (8-10 days after injection/day 1 of experiment, in bioluminescence range of 14-16) by tail vein or IP routes at doses ranging from 0.1 mg/kg to 50 mg/kg for 7 to 21 days. Optionally, the mice are imaged throughout the experiment every other day and survival monitored daily for the duration of the experiment. Expired mice are optionally subjected to necropsy at the end of the experiment. Another animal model is implantation into NOD-SCID mice of DoHH2, a cell line derived from human follicular lymphoma, that stably expresses luciferase. These in vivo tests optionally generate preliminary pharmacokinetic, pharmacodynamic and toxicology data.

### Clinical Trials.

To determine the suitability of the peptidomimetic macrocycles of the invention for treatment of humans, clinical trials are performed. For example, patients diagnosed with cancer and in need of treatment are selected and separated in treatment and one or more control groups, wherein the treatment group is administered a peptidomimetic macrocycle of the invention, while the control groups receive a placebo, a known anti-cancer drug, or the standard of care. The treatment safety and efficacy of the peptidomimetic macrocycles of the invention can thus be evaluated by performing comparisons of the patient groups with respect to factors such as survival and quality-of-life. In this example, the patient group treated with a peptidomimetic macrocyle show improved long-term survival compared to a patient control group treated with a placebo or the standard of care.

### Pharmaceutical Compositions and Routes of Administration

Methods of administration include but are not limited to intradermal, intramuscular, intraperitoneal, intravenous, subcutaneous, intranasal, epidural, oral, sublingual, intracerebral, intravaginal, transdermal, rectal, by inhalation, or topical by application to ears, nose, eyes, or skin.

The peptidomimetic macrocycles of the invention also include pharmaceutically acceptable derivatives or prodrugs thereof. A “pharmaceutically acceptable derivative” means any pharmaceutically acceptable salt, ester, salt of an ester, pro-drug or other derivative of a compound of this invention which, upon administration to a recipient, is capable of providing (directly or indirectly) a compound of this invention. For example, pharmaceutically acceptable derivatives may increase the bioavailability of the compounds of the invention when administered to a mammal (e.g., by increasing absorption into the blood of an orally administered compound) or which increases delivery of the active compound to a biological compartment (e.g., the brain or lymphatic system) relative to the parent species. Some pharmaceutically acceptable derivatives include a chemical group which increases aqueous solubility or active transport across the gastrointestinal mucosa.

In some embodiments, the peptidomimetic macrocycles of the invention are modified by covalently or non-covalently joining appropriate functional groups to enhance selective biological properties. Such modifications include those which increase biological penetration into a given biological compartment (e.g., blood, lymphatic system, central nervous system), increase oral availability, increase solubility to allow administration by injection, alter metabolism, and alter rate of excretion.

Pharmaceutically acceptable salts of the compounds of this invention include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts.

For preparing pharmaceutical compositions from the compounds of the present invention, pharmaceutically acceptable carriers include either solid or liquid carriers. Solid form preparations include powders, tablets, pills, capsules, cachets, suppositories, and dispersible granules. A solid carrier can be one or more substances, which also acts as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa.

In powders, the carrier is a finely divided solid, which is in a mixture with the finely divided active component. In tablets, the active component is mixed with the carrier having the necessary binding properties in suitable proportions and compacted in the shape and size desired.

Suitable solid excipients are carbohydrate or protein fillers include, but are not limited to sugars, including dextrose, lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents are added, such as the cross-linked polyvinyl pyrrolidone, agar, alginic acid, or a salt thereof, such as sodium alginate.

Liquid form preparations include solutions, suspensions, and emulsions, for example, water or water/propylene glycol solutions. For parenteral injection, liquid preparations can be formulated in solution in aqueous polyethylene glycol solution. The term “parenteral” as used herein refers modes of administration including intravenous, intraarterial, intramuscular, intraperitoneal, intrasternal, and subcutaneous.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

When the compositions of this invention comprise a combination of a peptidomimetic macrocycle and one or more additional therapeutic or prophylactic agents, both the compound and the additional agent should be present at dosage levels of between about 1 to 100%, and more preferably between about 5 to 95% of the dosage normally administered in a monotherapy regimen. In some embodiments, the additional agents are administered separately, as part of a multiple dose regimen, from the compounds of this invention. Alternatively, those agents are part of a single dosage form, mixed together with the compounds of this invention in a single composition.

### Methods of Use

In one aspect, the present invention provides novel peptidomimetic macrocycles that are useful in competitive binding assays to identify agents which bind to the natural ligand(s) of the proteins or peptides upon which the peptidomimetic macrocycles are modeled. For example, in the p53 MDM2 system, labeled stabilized peptidomimetic macrocyles based on the p53 is used in an MDM2 binding assay along with small molecules that competitively bind to MDM2. Competitive binding studies allow for rapid in vitro evaluation and determination of drug candidates specific for the p53/MDM2 system. Likewise in the BH3/BCL-X_{L} anti-apoptotic system labeled peptidomimetic macrocycles based on BH3 can be used in a BCL-X_{L} binding assay along with small molecules that competitively bind to BCL-X_{L}. Competitive binding studies allow for rapid in vitro evaluation and determination of drug candidates specific for the BH3/BCL-X_{L} system. The invention further provides for the generation of antibodies against the peptidomimetic macrocycles. In some embodiments, these antibodies specifically bind both the peptidomimetic macrocycle and the p53 or BH3 peptidomimetic precursors upon which the peptidomimetic macrocycles are derived. Such antibodies, for example, disrupt the p53/MDM2 or BH3/BCL-XL systems, respectively.

In other aspects, the present invention provides for both prophylactic and therapeutic methods of treating a subject at risk of (or susceptible to) a disorder or having a disorder associated with aberrant (e.g., insufficient or excessive) BCL-2 family member expression or activity (e.g., extrinsic or intrinsic apoptotic pathway abnormalities). It is believed that some BCL-2 type disorders are caused, at least in part, by an abnormal level of one or more BCL-2 family members (e.g., over or under expression), or by the presence of one or more BCL-2 family members exhibiting abnormal activity. As such, the reduction in the level and/or activity of the BCL-2 family member or the enhancement of the level and/or activity of the BCL-2 family member, is used, for example, to ameliorate or reduce the adverse symptoms of the disorder.

In another aspect, the present invention provides methods for treating or preventing hyperproliferative disease by interfering with the interaction or binding between p53 and MDM2 in tumor cells. These methods comprise administering an effective amount of a compound of the invention to a warm blooded animal, including a human, or to tumor cells containing wild type p53. In some embodiments, the administration of the compounds of the present invention induce cell growth arrest or apoptosis. In other or further embodiments, the present invention is used to treat disease and/or tumor cells comprising elevated MDM2 levels. Elevated levels of MDM2 as used herein refers to MDM2 levels greater than those found in cells containing more than the normal copy number (2) of mdm2 or above about 10,000 molecules of MDM2 per cell as measured by ELISA and similar assays (Picksley et al. (1994), Oncogene 9, 2523 2529).

As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

In some embodiments, the peptidomimetics macrocycles of the invention is used to treat, prevent, and/or diagnose cancers and neoplastic conditions. As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. A metastatic tumor can arise from a multitude of primary tumor types, including but not limited to those of breast, lung, liver, colon and ovarian origin. “Pathologic hyperproliferative” cells occur in disease states characterized by malignant tumor growth. Examples of non-pathologic hyperproliferative cells include proliferation of cells associated with wound repair. Examples of cellular proliferative and/or differentiative disorders include cancer, e.g., carcinoma, sarcoma, or metastatic disorders. In some embodiments, the peptidomimetics macrocycles are novel therapeutic agents for controlling breast cancer, ovarian cancer, colon cancer, lung cancer, metastasis of such cancers and the like.

Examples of cancers or neoplastic conditions include, but are not limited to, a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, or Kaposi sarcoma.

Examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus (1991), Crit Rev. Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Stemberg disease.

Examples of cellular proliferative and/or differentiative disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders of the lung include, but are not limited to, bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

Examples of cellular proliferative and/or differentiative disorders of the colon include, but are not limited to, non-neoplastic polyps, adenomas, familial syndromes, colorectal carcinogenesis, colorectal carcinoma, and carcinoid tumors.

Examples of cellular proliferative and/or differentiative disorders of the liver include, but are not limited to, nodular hyperplasias, adenomas, and malignant tumors, including primary carcinoma of the liver and metastatic tumors.

Examples of cellular proliferative and/or differentiative disorders of the ovary include, but are not limited to, ovarian tumors such as, tumors of coelomic epithelium, serous tumors, mucinous tumors, endometrioid tumors, clear cell adenocarcinoma, cystadenofibroma, Brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecomafibromas, androblastomas, hill cell tumors, and gonadoblastoma; and metastatic tumors such as Krukenberg tumors.

In other or further embodiments, the peptidomimetics macrocycles described herein are used to treat, prevent or diagnose conditions characterized by overactive cell death or cellular death due to physiologic insult, etc. Some examples of conditions characterized by premature or unwanted cell death are or alternatively unwanted or excessive cellular proliferation include, but are not limited to hypocellular/hypoplastic, acellular/aplastic, or hypercellular/hyperplastic conditions. Some examples include hematologic disorders including but not limited to fanconi anemia, aplastic anemia, thalaessemia, congenital neutropenia, myelodysplasia

In other or further embodiments, the peptidomimetics macrocycles of the invention that act to decrease apoptosis are used to treat disorders associated with an undesirable level of cell death. Thus, in some embodiments, the anti-apoptotic peptidomimetics macrocycles of the invention are used to treat disorders such as those that lead to cell death associated with viral infection, e.g., infection associated with infection with human immunodeficiency virus (HIV). A wide variety of neurological diseases are characterized by the gradual loss of specific sets of neurons, and the anti-apoptotic peptidomimetics macrocycles of the invention are used, in some embodiments, in the treatment of these disorders. Such disorders include Alzheimer's disease, Parkinson's disease, amyotrophic lateral sclerosis (ALS) retinitis pigmentosa, spinal muscular atrophy, and various forms of cerebellar degeneration. The cell loss in these diseases does not induce an inflammatory response, and apoptosis appears to be the mechanism of cell death. In addition, a number of hematologic diseases are associated with a decreased production of blood cells. These disorders include anemia associated with chronic disease, aplastic anemia, chronic neutropenia, and the myelodysplastic syndromes. Disorders of blood cell production, such as myelodysplastic syndrome and some forms of aplastic anemia, are associated with increased apoptotic cell death within the bone marrow. These disorders could result from the activation of genes that promote apoptosis, acquired deficiencies in stromal cells or hematopoietic survival factors, or the direct effects of toxins and mediators of immune responses. Two common disorders associated with cell death are myocardial infarctions and stroke. In both disorders, cells within the central area of ischemia, which is produced in the event of acute loss of blood flow, appear to die rapidly as a result of necrosis. However, outside the central ischemic zone, cells die over a more protracted time period and morphologically appear to die by apoptosis. In other or further embodiments, the anti-apoptotic peptidomimetics macrocycles of the invention are used to treat all such disorders associated with undesirable cell death.

Some examples of immunologic disorders that are treated with the peptidomimetics macrocycles described herein include but are not limited to organ transplant rejection, arthritis, lupus, IBD, Crohn's disease, asthma, multiple sclerosis, diabetes, etc.

Some examples of neurologic disorders that are treated with the peptidomimetics macrocycles described herein include but are not limited to Alzheimer's Disease, Down's Syndrome, Dutch Type Hereditary Cerebral Hemorrhage Amyloidosis, Reactive Amyloidosis, Familial Amyloid Nephropathy with Urticaria and Deafness, Muckle-Wells Syndrome, Idiopathic Myeloma; Macroglobulinemia-Associated Myeloma, Familial Amyloid Polyneuropathy, Familial Amyloid Cardiomyopathy, Isolated Cardiac Amyloid, Systemic Senile Amyloidosis, Adult Onset Diabetes, Insulinoma, Isolated Atrial Amyloid, Medullary Carcinoma of the Thyroid, Familial Amyloidosis, Hereditary Cerebral Hemorrhage With Amyloidosis, Familial Amyloidotic Polyneuropathy, Scrapie, Creutzfeldt-Jacob Disease, Gerstmann Straussler-Scheinker Syndrome, Bovine Spongiform Encephalitis, a prion-mediated disease, and Huntington's Disease.

Some examples of endocrinologic disorders that are treated with the peptidomimetics macrocycles described herein include but are not limited to diabetes, hypothyroidism, hypopituitarism, hypoparathyroidism, hypogonadism, etc.

Examples of cardiovascular disorders (e.g., inflammatory disorders) that are treated or prevented with the peptidomimetics macrocycles of the invention include, but are not limited to, atherosclerosis, myocardial infarction, stroke, thrombosis, aneurism, heart failure, ischemic heart disease, angina pectoris, sudden cardiac death, hypertensive heart disease; non-coronary vessel disease, such as arteriolosclerosis, small vessel disease, nephropathy, hypertriglyceridemia, hypercholesterolemia, hyperlipidemia, xanthomatosis, asthma, hypertension, emphysema and chronic pulmonary disease; or a cardiovascular condition associated with interventional procedures (“procedural vascular trauma”), such as restenosis following angioplasty, placement of a shunt, stent, synthetic or natural excision grafts, indwelling catheter, valve or other implantable devices. Preferred cardiovascular disorders include atherosclerosis, myocardial infarction, aneurism, and stroke.

### EXAMPLES

The following section provides illustrative examples of the present invention.

### Example 1

### Preparation of Alpha,Alpha-Disubstituted Amino Acids

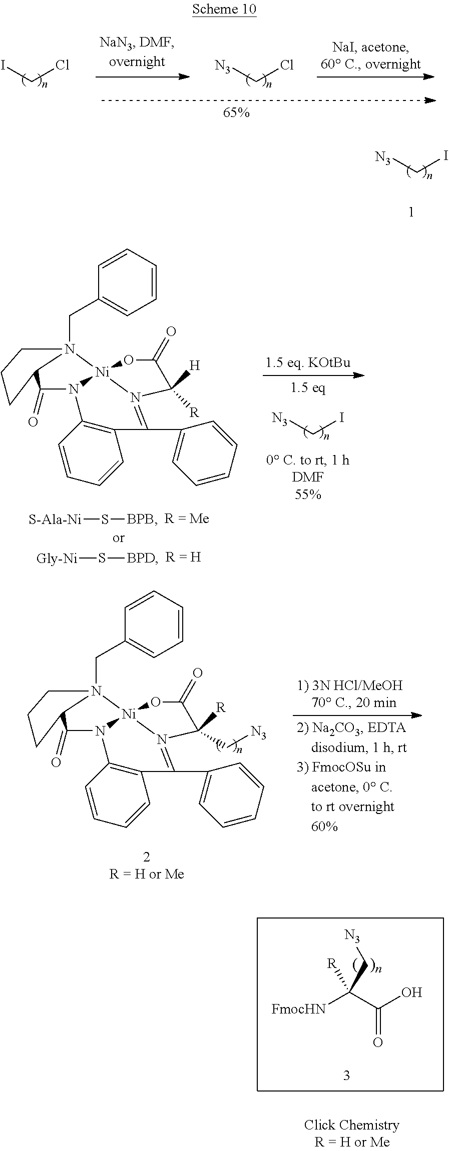

1-Azido-n-iodo-alkanes 1. To 1-iodo-n-chloro-alkane (8.2 mmol) in DMF (20 ml) was added sodium azide (1.2 eq.) and the reaction mixture was stirred at ambient temperature overnight. The reaction mixture was then diluted with diethyl ether and water. The organic layer was dried over magnesium sulfate and concentrated in vacuo to give 1-azido-n-chloro-alkane. The azide was diluted with acetone (40 ml) and sodium iodide (1.5 eq.) was added. The solution was heated at 60° C. overnight. Afterwards, the reaction mixture was diluted with water and the product was extracted with diethyl ether. The organic layer was dried over magnesium sulfate and concentrated in vacuo. The product 1 was purified by passing it through a plug of neutral alumina. Overall yield: 65%. 1-Azido-3-iodo-propane: ¹H NMR (CDCl₃) δ: 2.04 (q, 2H, CH₂); 3.25 (t, 2H, CH₂I); 3.44 (t, 2H, CH₂N₃). 1-Azido-5-iodo-pentane: ¹H NMR (CDCl₃) δ: 1.50 (m, 2H, CH₂); 1.62 (m, 2H, CH₂); 1.86 (m, 2H, CH₂); 3.19 (t, 2H, CH₂I); 3.29 (t, 2H, CH₂N₃).

αMe-Sn-azide-Ni—S—BPB (R=Me), 2. To S-Ala-Ni—S—BPB (10.0 mmol) and KO-tBu (1.5 eq.) was added 45 mL of DMF under argon. The compound 1 (1.5 eq.) in solution of DMF (4.0 mL) was added via syringe. The reaction mixture was stirred at ambient temperature for 1 h. The solution was then quenched with 5% aqueous acetic acid and diluted with water. The oily product was collected by filtration and washed with water. The desired product 2 was purified by flash chromatography on normal phase using acetone and dichloromethane as eluents to give a red solid in 55% yield. αMe-S3-azide-Ni—S—BPB (2, R=Me, n=3): M+H calc. 595.19. M+H obs. 595.16; ¹H NMR (CDCl₃) δ: 1.25 (s, 3H, Me (αMe-S3-azide)); 1.72-1.83 (m, 2H, CH₂); 2.07 (m, 2H, CH₂); 2.17 (m, 1H, CH₂); 2.48 (m, 2H, CH₂); 2.67 (m, 1H, CH₂); 3.27 (m, 2H, CH₂); 3.44 (m, 2H, CH₂); 3.64 (m, 1H, CH_{α}); 3.68 and 4.47 (AB system, 2H, CH₂ (benzyl), J=12.8 Hz); 6.62-6.64 (m, 2H); 7.05 (d, 1H); 7.13 (m, 1H); 7.30 (m, 2H); 7.28-7.32 (m, 2H); 7.38-7.42 (m, 3H); 7.47-7.50 (m, 2H); 8.01 (d, 1H); 8.07 (m, 2H).

Sn-azide-Ni—S—BPB (R=H), 2. To Gly-Ni—S—BPB (10.0 mmol) and KO-tBu (1.5 eq.) was added 45 mL of DMF under argon. The compound 1 (1.5 eq.) in solution of DMF (4.0 mL) was added via syringe. The reaction mixture was stirred at ambient temperature for 1 h. The solution was then quenched with 5% aqueous acetic acid and diluted with water. The oily product was collected by filtration and washed with water. The desired product 2 was purified by flash chromatography on normal phase using acetone and dichloromethane as eluents to give a red solid in 55% yield. S3-azide-Ni—S—BPB (2, R=H, n=3): M+H calc. 581.17. M+H obs. 581.05; ¹H NMR (CDCl₃) δ: 1.72 (m, 2H, CH₂); 2.07 (m, 1H, CH₂); 2.16 (m, 3H, CH₂); 2.53 (m, 1H, CH₂); 2.75 (m, 1H, CH₂); 3.08 (m, 1H, CH₂); 3.22 (m, 1H, CH₂); 3.49 (m, 2H, CH₂); 3.59 (m, CH_{α}); 3.58 and 4.44 (AB system, 2H, CH₂ (benzyl)); 3.87 (m, CH_{α}); 6.64 (m, 2H); 6.96 (d, 1H); 7.14-7.19 (m, 2H); 7.35 (m, 2H); 7.51 (m, 4H); 8.04 (d, 2H); 8.12 (d, 1H).

Fmoc-αMe-Sn-azide-OH (R=Me), 3. To a solution of 3N HCl/MeOH (1/1, 12 mL) at 70° C. was added a solution of compound 2 (1.65 mmol) in MeOH (3 ml) dropwise. The starting material disappeared within 10-20 min. The green reaction mixture was then concentrated in vacuo. The crude residue was diluted with 10% aqueous Na₂CO₃ (16 ml) and cooled to 0° C. with an ice bath. Fmoc-OSu (1.1 eq.) dissolved in acetone (16 ml) was added and the reaction was allowed to warm up to ambient temperature with stirring overnight. Afterwards, the reaction was diluted with ethyl acetate and 1 N HCl. The organic layer was washed with 1 N HCl (3×). The organic layer was then dried over magnesium sulfate and concentrated in vacuo. The desired product 3 was purified on normal phase using methanol and dichloromethane as eluents to give a viscous oil in 36% overall yield for both steps. Fmoc-αMe-S3-azide-OH (2, R=Me, n=3): M+H calc. 395.16. M+H obs. 395.12; ¹H NMR (CDCl₃) δ: 0.85 (bs, 1H, CH₂); 1.10 (bs, 1H, CH₂); 1.61 (s, 3H, Me (αMe-S3-azide)); 1.98 (bs, 1H, CH₂); 2.22 (bs, 1H, CH₂); 3.27 (bs, 2H, CH₂); 4.21 (m, 1H, CH); 4.42 (bs, 2H, CH₂); 5.53 (s, 1H, NH); 7.33 (m, 2H); 7.40 (m, 2H); 7.57 (m, 2H); 7.77 (d, 2H).

Fmoc-Sn-azide-OH (R=H), 3. To a solution of 3N HCl/MeOH (1/1, 12 mL) at 70° C. was added a solution of compound 2, R=H (1.65 mmol) in MeOH (3 ml) dropwise. The starting material disappeared within 10-20 min. The green reaction mixture was then concentrated in vacuo. The crude residue was diluted with 10% aqueous Na₂CO₃ (16 ml) and cooled to 0° C. with an ice bath. Fmoc-OSu (1.1 eq.) dissolved in acetone (16 ml) was added and the reaction was allowed to warm up to ambient temperature with stirring overnight. Afterwards, the reaction was diluted with ethyl acetate and 1 N HCl. The organic layer was washed with 1 N HCl (3×). The organic layer was then dried over magnesium sulfate and concentrated in vacuo. The desired product 3 was purified on normal phase using methanol and dichloromethane as eluents to give a viscous oil in 36% overall yield for both steps. Fmoc-S3-azide-OH (2, R=H, n=3): M+H calc. 381.15. M+H obs. 381.07; ¹H NMR (CDCl₃) 1.66 (bs, 2H, CH₂); 1.78 (bs, 1H, CH₂); 1.99 (bs, 1H, CH2); 3.12 (1H, CH_{α}); 3.32 (bs, 2H, CH₂); 4.21 (m, 1H, CH); 4.43 (bs, 2H, CH₂); 5.37 (s, 1H, NH); 7.31 (m, 2H); 7.40 (m, 2H); 7.58 (m, 2H); 7.77 (d, 2H).

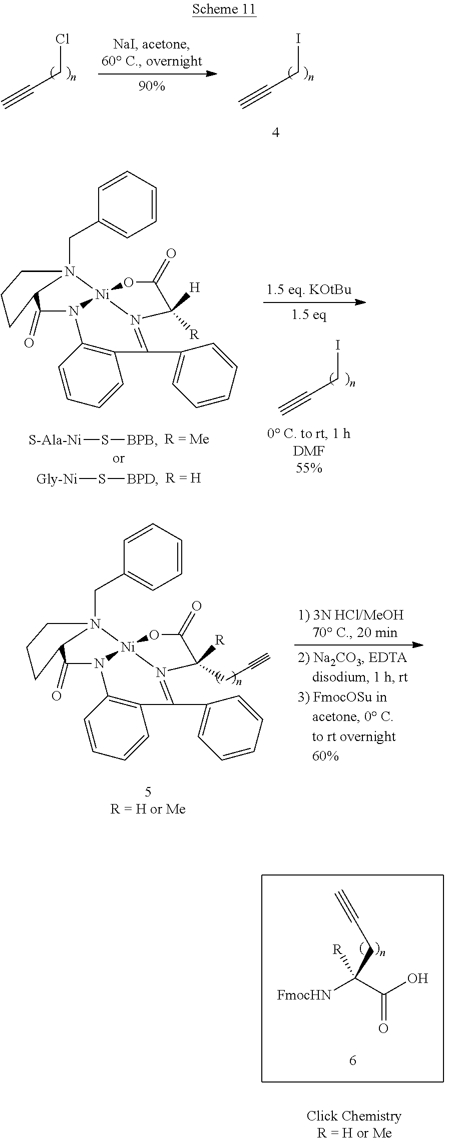

(n+2)-Iodo-1-alkyne, 4. To a solution of (n+2)-chloro-1-alkyne (47.8 mmol) in acetone (80 mL) was added sodium iodide (2 eq.). The reaction was heated at 60° C. overnight. Afterwards, the reaction was diluted with water and the product was extracted with diethyl ether. The organic layer was dried over magnesium sulfate and concentrated in vacuo. The product 5 was purified by passing it through a plug of neutral alumina. Yield: 92%. 5-Iodo-1-alkyne (n=3): ¹H NMR (CDCl₃) 2.00 (m, 3H, CH₂+CH); 2.34 (m, 2H, CH₂); 3.31 (t, 2H, CH₂).

αMe-S(n+2)-alkyne-Ni—S—BPB (R=Me), 5. To S-Ala-Ni—S—BPB (10.0 mmol) and KO-tBu (1.5 eq.) was added 45 mL of DMF under argon. The compound 4 (1.5 eq.) in solution of DMF (4.0 mL) was added via syringe. The reaction was stirred at ambient temperature for 1 h. The reaction was then quenched with 5% aqueous acetic acid and diluted with water. The oily product was collected by filtration and washed with water. The desired product 5 was purified by flash chromatography on normal phase using acetone and dichloromethane as eluents to give a red solid in 55% yield. αMe-S5-alkyne-Ni—S—BPB (5, R=Me, n=3): M+H calc. 578.19. M+H obs. 578.17; ¹H NMR (CDCl₃) δ: 1.21 (s, 3H, Me (αMe-S5-alkyne)); 1.62 (1H, CH, acetylene); 1.77 (m, 1H, CH₂); 1.92 (m, 1H, CH₂); 2.05 (m, 2H, CH₂); 2.21 (m, 2H, CH₂); 2.33 (m, 1H, CH₂); 2.51 (m, 2H, CH₂); 2.70 (m, 1H, CH₂); 3.23 (m, 1H, CH_{α}); 3.44 (m, 1H, CH₂); 3.66 (m, 1H, CH₂); 3.69 and 4.49 (AB system, 2H, CH₂ (benzyl)); 6.64 (m, 2H); 7.05-7.13 (m, 2H); 7.27-7.31 (m, 2H); 7.40 (m, 3H); 7.47 (m, 2H); 8.00 (d, 1H); 8.06 (m, 2H).

S(n+2)-alkyne-Ni—S—BPB (R=H), 5. To Gly-Ni—S—BPB (10.0 mmol) and KO-tBu (1.5 eq.) was added 45 mL of DMF under argon. The compound 4 (1.5 eq.) in solution of DMF (4.0 mL) was added via syringe. The reaction was stirred at ambient temperature for 1 h. The reaction was then quenched with 5% aqueous acetic acid and diluted with water. The oily product was collected by filtration and washed with water. The desired product 5 was purified by flash chromatography on normal phase using acetone and dichloromethane as eluents to give a red solid in 55% yield. S5-alkyne-Ni—S—BPB (5, R=H, n=3): M+H calc. 564.17. M+H obs. 564.15; ¹H NMR (CDCl₃) δ: 1.75 (m, 2H, CH₂); 1.95 (m, 1H, CH, acetylene); 2.06 (m, 2H, CH₂); 2.16 (m, 2H, CH₂); 2.30 (m, 1H, CH₂); 2.52 (m, 1H, CH₂); 2.77 (m, 1H, CH₂); 3.49 (m, 2H, CH₂); 3.59 (m, 1H, CH_{α}); 3.88 (m, 1H, CH_{α}); 3.58 and 4.43 (AB system, 2H, CH₂ (benzyl)); 6.63 (m, 2H); 6.96 (d, 1H); 7.14-7.19 (m, 2H); 7.34 (m, 2H); 7.44 (m, 1H); 7.49 (m, 3H); 8.05 (d, 2H); 8.12 (d, 1H).

Fmoc-αMe-S(n+2)-alkyne-OH (R=Me), 6. To a solution of 3N HCl/MeOH (1/1, 18 mL) at 70° C. was added a solution of compound 5, R=Me (2.4 mmol) in MeOH (4 ml) dropwise. The starting material disappeared within 5-10 min. The green solution was then concentrated in vacuo. The crude residue was diluted with 10% aqueous Na₂CO₃ (24 ml) cooled to 0° C. with an ice bath. Fmoc-OSu (1.1 eq.) dissolved in dioxane (24 ml) was added and the reaction was allowed to warm up to ambient temperature with stirring overnight. Afterwards, the reaction was diluted with ethyl acetate and 1 N HCl. The organic layer was washed with 1 N HCl (3×). The organic layer was then dried over magnesium sulfate and concentrated in vacuo. The desired product 6 was isolated after flash chromatography purification on silica gel using methanol and dichloromethane as eluents to give viscous oil that solidifies upon standing in 60% yield. Fmoc-αMe-S5-alkyne-OH (6, R=Me, n=3): M+H calc. 378.16. M+H obs. 378.15; ¹H NMR (CDCl₃) δ: 1.42 (bs, 1H, CH₂); 1.54 (bs, 1H, CH₂); 1.61 (s, 3H, Me (αMe-S3-azide)); 1.96 (bs, 2H, CH₂); 2.20 (bs, 3H, CH₂+CH acetylene); 4.21 (m, 1H, CH); 4.42 (bs, 2H, CH₂); 5.51 (s, 1H, NH); 7.32 (m, 2H); 7.40 (m, 2H); 7.59 (d, 2H); 7.77 (d, 2H).

Fmoc-S(n+2)-alkyne-OH (R=H), 6. To a solution of 3N HCl/MeOH (1/1, 18 mL) at 70° C. was added a solution of compound 5, R=H (2.4 mmol) in MeOH (4 ml) dropwise. The starting material disappeared within 5-10 min. The green solution was then concentrated in vacuo. The crude residue was diluted with 10% aqueous Na₂CO₃ (24 ml) cooled to 0° C. with an ice bath. Fmoc-OSu (1.1 eq.) dissolved in dioxane (24 ml) was added and the reaction was allowed to warm up to ambient temperature with stirring overnight. Afterwards, the reaction was diluted with ethyl acetate and 1 N HCl. The organic layer was washed with 1 N HCl (3×). The organic layer was then dried over magnesium sulfate and concentrated in vacuo. The desired product 6 was isolated after flash chromatography purification on silica gel using methanol and dichloromethane as eluents to give viscous oil that solidifies upon standing in 60% yield. Fmoc-S5-alkyne-OH (6, R=H, n=3): M+H calc. 364.15. M+H obs. 364.14; ¹H NMR (CDCl₃) δ: 1.48-1.62 (m, 3H, CH₂); 1.81 (m, 1H, CH₂); 1.98 (m, 1H, CH₂); 1.99-2.11 (m, 1H, CH₂); 2.24 (m, 1H, CH acetylene); 4.21 (m, 1H, CH); 4.42 (bs, 2H, CH₂); 5.51 (s, 1H, NH); 7.32 (m, 2H); 7.40 (m, 2H); 7.59 (d, 2H); 7.77 (d, 2H).

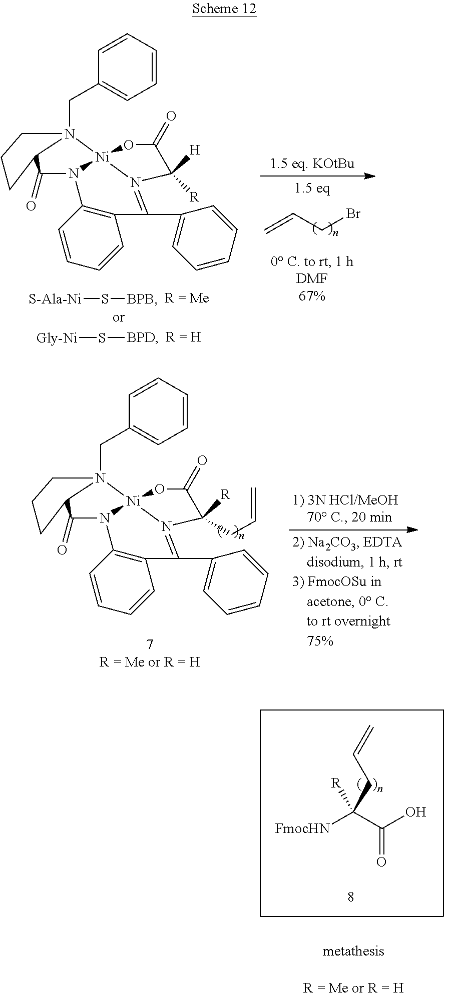

αMe-S(n+2)-alkene-Ni—S—BPB (R=Me), 7. To S-Ala-Ni—S—BPB (10.0 mmol) and KO-tBu (2 eq.) was added 45 mL of DMF under argon. 1-Bromo-n-alkene (1.5 eq.) in solution of DMF (4.0 mL) was added via syringe. The reaction was stirred at ambient temperature for 1 h. The reaction was then quenched with 5% aqueous acetic acid and diluted with water. The oily product was collected by filtration and washed with water. The desired product 7 was purified by flash chromatography on normal phase using acetone and dichloromethane as eluents to give a red solid in 55% yield. αMe-S5-alkene-Ni—S—BPB (7, R=Me, n=3): M+H calc. 580.20. M+H obs. 580.17; ¹H NMR (CDCl₃) δ: 1.23 (s, 3H, Me (αMe-S5-alkene)); 1.69 (m, 3H, CH₂); 2.0-2.14 (m, 5H, CH₂); 2.37-2.53 (m, 1H, CH₂); 2.69 (m, 1H, CH₂); 3.26 (m, 1H, CH₂); 3.43 (m, 1H, CH₂); 3.64 (m, 1H, CH_{α}); 3.70 and 4.50 (AB system, 2H, CH₂ (benzyl), J=12.8 Hz); 5.0-5.10 (m, 2H, CH₂ alkene); 5.85 (m, 1H, CH alkene); 6.63 (m, 2H); 6.96 (d, 1H); 7.12 (m, 1H); 7.27-7.32 (m, 2H); 7.38-7.42 (m, 3H); 7.47-7.50 (m, 2H); 7.99 (d, 1H); 8.06 (m, 2H). αMe-S8-alkene-Ni—S—BPB (7, R=Me, n=6): M+H calc. 622.25. M+H obs. 622.22; ¹H NMR (CDCl₃) δ: 1.24 (s, 3H, Me (αMe-S8-alkene)); 1.29-1.44 (m, 5H, CH₂); 1.56-1.74 (m, 3H, CH₂); 2.06 (m, 5H, CH₂); 2.32-2.51 (m, 2H, CH₂); 2.68 (m, 1H, CH₂); 3.28 (m, 1H, CH₂); 3.42 (m, 1H, CH₂); 3.62 (m, 1H, CH_{α}); 3.70 and 4.50 (AB system, 2H, CH₂ (benzyl), J=12.8 Hz); 4.92-5.02 (m, 2H, CH₂ alkene); 5.76-5.85 (m, 1H, CH alkene); 6.63 (m, 2H); 6.96 (d, 1H); 7.12 (m, 1H); 7.27-7.33 (m, 2H); 7.38-7.42 (m, 3H); 7.45-7.51 (m, 2H); 7.99 (d, 1H); 8.06 (m, 2H).

To Gly-Ni—S—BPB (10.0 mmol) and KO-tBu (2 eq.) was added 45 mL of DMF under argon. 1-Bromo-n-alkene (1.5 eq.) in solution of DMF (4.0 mL) was added via syringe. The reaction was stirred at ambient temperature for 1 h. The reaction was then quenched with 5% aqueous acetic acid and diluted with water. The oily product was collected by filtration and washed with water. The desired product 7 was purified by flash chromatography on normal phase using acetone and dichloromethane as eluents to give a red solid in 55% yield. S5-alkene-Ni—S—BPB (7, R=H, n=3): M+H calc. 566.19, M+H obs. 566.17; ¹H NMR (CDCl₃) δ: 1.69 (m, 3H, CH₂); 1.90-2.23 (m, 5H, CH₂); 2.52 (m, 1H, CH₂); 2.75 (m, 1H, CH₂); 3.44-3-49 (m, 2H, CH₂); 3.50 (m, 1H, CH_{α}); 3.90 (m, 1H, CH_{α}′); 3.58 and 4.44 (AB system, 2H, CH₂ (benzyl)); 4.97 (m, 2H, CH₂ alkene); 5.72 (m, 1H, CH alkene); 6.64 (m, 2H); 6.91 (d, 1H); 7.14-7.20 (m, 2H); 7.34 (m, 2H); 7.44-7.49 (m, 4H); 8.04 (d, 2H); 8.12 (d, 1H).

Fmoc-αMe-S(n+2)-alkene-OH (R=Me), 8. To a solution (18 mL) of 1/1 3N HCl/MeOH at 70° C. was added a solution of compound 7, R=Me (2.4 mmol) in MeOH (4 ml) dropwise. The starting material disappeared within 5-10 min. The green solution was then concentrated in vacuo. The crude residue was diluted with 10% aqueous Na₂CO₃ (24 ml) cooled to 0° C. with an ice bath. Fmoc-OSu (1.1 eq.) dissolved in acetone (24 ml) was added and the reaction was allowed to warm up to ambient temperature with stirring overnight. Afterwards, the reaction was diluted with ethyl acetate and 1 N HCl. The organic layer was washed with 1 N HCl (3×). The organic layer was then dried over magnesium sulfate and concentrated in vacuo. The desired product 8 was isolated after flash chromatography purification on normal phase using methanol and dichloromethane as eluents to give viscous oil that solidifies upon standing in 75% yield. Fmoc-αMe-S5-alkene-OH (8, R=Me, n=3): M+H calc. 380.18. M+H obs. 380.16; ¹H NMR (CDCl₃) δ: 1.26-1.41 (m, 3H, CH₂); 1.61 (bs, 3H, αMe); 1.86 (bs, 1H); 2.05 (m, 2H, CH₂); 4.22 (m, 1H, CH (Fmoc)); 4.40 (bs, 2H, CH₂(Fmoc)); 4.97 (m, 2H, CH₂ alkene); 5.53 (bs, 1H, NH); 5.75 (m, 1H, CH alkene); 7.29-7.33 (m, 2H); 7.38-7.42 (m, 2H); 7.59 (d, 2H); 7.76 (d, 2H). Fmoc-αMe-S8-alkene-OH (8, R=Me, n=6): M+H calc. 422.23. M+H obs. 422.22; ¹H NMR (CDCl₃) δ: 1.28 (m, 9H, CH₂); 1.60 (bs, 3H, αMe); 1.83 (bs, 1H); 2.01 (m, 2H, CH₂); 4.22 (m, 1H, CH (Fmoc)); 4.39 (bs, 2H, CH₂(Fmoc)); 4.90-5.00 (m, 2H, CH₂ alkene); 5.49 (bs, 1H, NH); 5.75-5.82 (m, 1H, CH alkene); 7.29-7.33 (m, 2H); 7.38-7.42 (m, 2H); 7.59 (d, 2H); 7.77 (d, 2H).

Fmoc-S(n+2)-alkene-OH (R=H), 8. To a solution (18 mL) of 1/1 3N HCl/MeOH at 70° C. was added a solution of compound 7, R=H (2.4 mmol) in MeOH (4 ml) dropwise. The starting material disappeared within 5-10 min. The green solution was then concentrated in vacuo. The crude residue was diluted with 10% aqueous Na₂CO₃ (24 ml) cooled to 0° C. with an ice bath. Fmoc-OSu (1.1 eq.) dissolved in acetone (24 ml) was added and the reaction was allowed to warm up to ambient temperature with stirring overnight. Afterwards, the reaction was diluted with ethyl acetate and 1 N HCl. The organic layer was washed with 1 N HCl (3×). The organic layer was then dried over magnesium sulfate and concentrated in vacuo. The desired product 8 was isolated after flash chromatography purification on normal phase using methanol and dichloromethane as eluents to give viscous oil that solidifies upon standing in 75% yield. Fmoc-S5-alkene-OH (8, R=H, n=3): M+H calc. 365.16. M+H obs. 365.09; ¹H NMR (CDCl₃) δ: 1.48 (m, 2H, CH₂); 1.72 (m, 1H); 1.91 (m, 1H, CH₂); 2.09 (m, 2H); 4.23 (m, 1H, CH (Fmoc)); 4.42 (m, 2H, CH₂ (Fmoc)); 5.00 (m, 3H, CH₂ alkene+CH_{α}); 5.22 (d, 1H, NH); 5.76 (m, 1H, CH alkene); 7.31 (m, 2H); 7.40 (m, 2H); 7.59 (d, 2H); 7.76 (d, 2H).

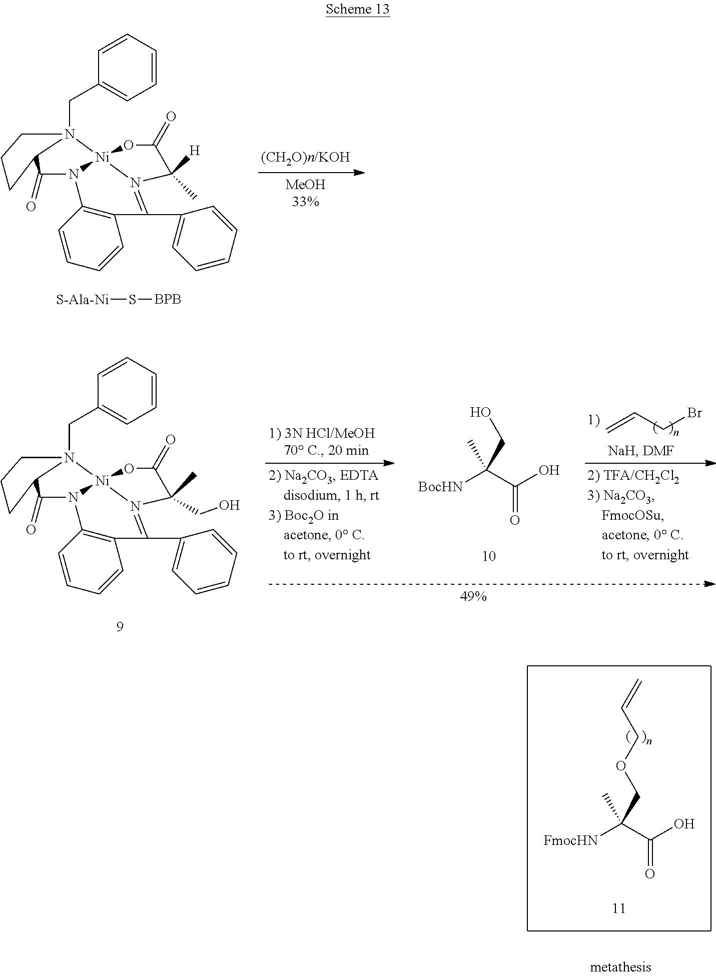

αMe-S-Ser-Ni—S—BPB, 9. To a solution of KOH (7.5 eq.) in methanol (20 mL) were added S-Ala-Ni—S—BPB (4 mmol) and paraformaldehyde (20 eq.) at room temperature. The reaction mixture was stirred overnight and neutralized with acetic acid. Then water was added to precipitate a mixture of diastereoisomers. Precipitation was completed overnight. The precipitate was filtered off, washed with water and dried under vacuum. The diastereoisomer (S, S), 9 were isolated by flash chromatography on normal phase using acetone and dichloromethane as eluents. The compound 9 is a red solid (yield 33%). M+H calc. 542.15. M+H obs. 542.09; ¹H NMR (CDCl₃) δ: 1.05 (s, 3H, Me (serine)); 1.98 (m, 2H, CH₂); 2.39 (m, 1H, CH₂); 2.65 (m, 1H, CH₂); 3.41 (m, 2H, CH₂); 3.44 (m, 1H, CH_{α}); 3.69 (m, 2H, CH₂ (serine)); 3.58 and 4.37 (AB system, 2H, CH₂ (benzyl), J=Hz); 6.60 (m, 1H); 6.67 (dd, 1H); 7.1 (m, 1H); 7.17 (d, 1H); 7.27 (m, 2H); 7.35-7.47 (m, 5H); 7.95 (dd, 1H); 8.09 (m, 2H).

Boc-αMe-L-Ser-OH, 10. To a solution of 3N HCl/MeOH (1/1, 6 ml) at 70° C. was added 0.86 mmol of compound 10 (dissolved in 2 ml MeOH). The solution was stirred at 70° C. for 15-20 min till the red color disappeared. The green solution was then concentrated to dryness. Water (3 ml) was added dropwise to precipitate the HCl salt of BPB auxiliary. The filtrate was removed and the white solid was washed twice with 1.5 ml water each (85% recovery of BPB, HCl). To the combined filtrates were added 8 eq. of solid Na₂CO₃, followed by 2 eq EDTA disodium salt. The reaction was stirred at room temperature for 1 h. The solution became blue. Then it was cooled to 0° C. with ice/water bath and 1.1 eq. of Boc₂O (dissolved in 6 ml dioxane) was added dropwise. The reaction was stirred overnight. Afterwards it was diluted with diethyl ether and water. The water layer was extracted once with diethyl ether. The aqueous layer was acidified with 1N HCl to pH=3 and washed with diethyl ether (3×). The combined organic layers were washed with brine, dried over MgSO₄ and concentrated in vacuo. The Boc protecting amino acid was used with any further purification for the next step. M+H calc. 260.14, M+H obs. 260.12; ¹H NMR (CDCl₃) δ: 1.45 (s, 9H, Boc); 1.50 (s, 3H, αMe (serine)); 3.86 (m, 2H, CH₂); 5.48 (s, 1H, NH).

Fmoc-αMe-L-Ser(OAllyl)-OH (n=1), 11. To a solution of 10 (2 mmol) in DMF (10 ml) at 0° C. were added NaH (2 eq.) and allyl bromide (1 eq.). The solution was stirred at 0° C. for 2 h. The reaction was diluted with ethyl acetate and water. The organic layer was washed with brine, dried over MgSO₄ and concentrated in vacuo. The crude material was dissolved in dichloromethane (6 mL) and TFA (3 mL) was added to the solution. The reaction was stirred for 1 h. The solution was then concentrated to dryness. Finally the crude material was dissolved in solution of aqueous NaHCO₃ and acetone (1/1, 20 mL) and FmocOSu (1.1 eq.) was added dropwise at 0° C. The reaction was stirred overnight. Afterwards the solution mixture was diluted with diethyl ether and water. The organic layer were washed with brine, dried over MgSO₄ and concentrated in vacuo. The desired product 11 was isolated after flash chromatography purification on silica gel using methanol and dichloromethane as eluents to give viscous oil in 49% yield. M+H calc. 382.16. M+H obs. 382.14; ¹H NMR (CDCl₃) δ: 1.62 (s, 3H, αMe (serine)); 3.80 (bs, 2H, CH₂); 4.02 (bs, 2H, CH₂); 4.24 (m, 1H, CH); 4.40 (bs, 2H, CH₂); 5.23 (m, 2H, CH₂); 5.74 (s, 1H, NH); 5.84 (m, 1H, CH); 7.32 (m, 2H); 7.40 (m, 2H); 7.60 (d, 2H); 7.76 (d, 2H).

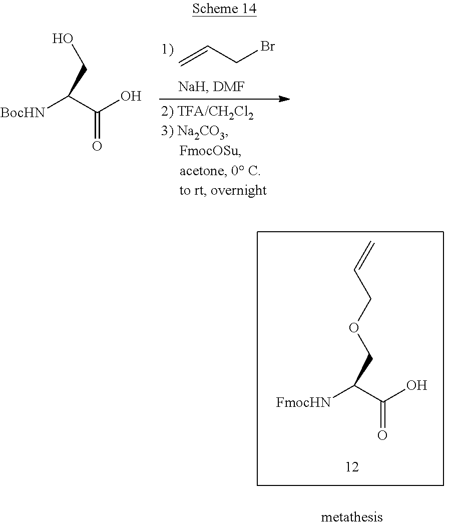

Fmoc-L-Ser(OAllyl)-OH, 12. To a solution of Boc-L-Serine (2 mmol) in DMF (10 ml) at 0° C. were added NaH (2 eq.) and allyl bromide (1 eq.). The solution was stirred at 0° C. for 2 h. The reaction was diluted with ethyl acetate and water. The organic layer was washed with brine, dried over MgSO₄ and concentrated in vacuo. The crude material was dissolved in dichloromethane (6 mL) and TFA (3 mL) was added to the solution. The reaction was stirred for 1 h. The solution was then concentrated to dryness. Finally the crude material was dissolved in solution of aqueous NaHCO₃ and acetone (1/1, 20 mL) and FmocOSu (1.1 eq.) was added dropwise at 0° C. The reaction was stirred overnight. Afterwards the solution mixture was diluted with diethyl ether and water. The organic layer were washed with brine, dried over MgSO₄ and concentrated in vacuo. The desired product 12 was isolated after flash chromatography purification on silica gel using methanol and dichloromethane as eluents to give viscous oil in 69% yield. M+H calc. 367.14. M+H obs. 367.12; ¹H NMR (CDCl₃) δ: 3.64 (m, 1H, CH_{α}); 3.88 (m, 1H, CH Fmoc); 3.96 (m, 2H, CH₂Fmoc); 4.17 (m, 1H, CH₂); 4.36 (m, 2H, CH₂); 4.48 (m, 1H, CH₂); 5.14 (m, 2H, CH₂); 5.60 (d, 1H, NH); 5.79 (m, 1H, CH); 7.24 (m, 2H); 7.33 (m, 2H); 7.54 (m, 2H); 7.68 (d, 2H).

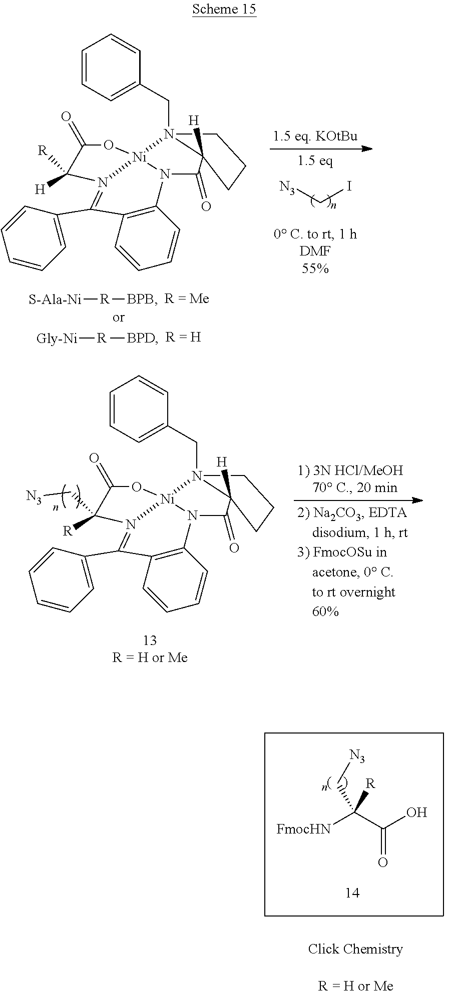

αMe-Rn-azide-Ni—R—BPB (R=Me), 13. To R-Ala-Ni—R—BPB (10.0 mmol) and KO-tBu (1.5 eq.) was added 45 mL of DMF under argon. The compound 1 (1.5 eq.) in solution of DMF (4.0 mL) was added via syringe. The reaction mixture was stirred at ambient temperature for 1 h. The solution was then quenched with 5% aqueous acetic acid and diluted with water. The oily product was collected by filtration and washed with water. The desired product 13 was purified by flash chromatography on normal phase using acetone and dichloromethane as eluents to give a red solid in 55% yield. αMe-R5-azide-Ni—R—BPB (13, R=Me, n=5): M+H calc. 623.22. M+H obs. 623.19; ¹H NMR (CDCl₃) δ: 1.24 (s, 3H, Me (αMe-R5-azide)); 1.33 (m, 2H, CH₂); 1.63 (m, 4H, CH₂); 2.05 (m, 3H, CH₂); 2.32 (m, 1H, CH₂); 2.48 (m, 1H, CH₂); 2.67 (m, 1H, CH₂); 3.28 (m, 3H, CH₂); 3.43 (m, 1H, CH₂); 3.63 (m, 1H, CH_{α}); 3.71 and 4.50 (AB system, 2H, CH₂ benzyl); 6.64 (m, 2H); 6.95 (d, 1H); 7.13 (m, 1H); 7.28-7.32 (m, 2H); 7.38-7.42 (m, 3H); 7.47-7.50 (m, 2H); 7.99 (d, 1H); 8.06 (d, 2H). αMe-R6-azide-Ni—R—BPB (13, R=Me, n=6): M+H calc. 637.24. M+H obs. 637.22; ¹H NMR (CDCl₃) δ: 1.24 (s, 3H, Me (αMe-R6-azide)); 1.33 (m, 2H, CH₂); 1.48 (m, 2H, CH₂); 1.63 (m, 4H, CH₂); 2.05 (m, 3H, CH₂); 2.32 (m, 1H, CH₂); 2.48 (m, 1H, CH₂); 2.67 (m, 1H, CH₂); 3.28 (m, 3H, CH₂); 3.43 (m, 1H, CH₂); 3.63 (m, 1H, CH_{α}); 3.71 and 4.50 (AB system, 2H, CH₂ benzyl); 6.64 (m, 2H); 6.95 (d, 1H); 7.13 (m, 1H); 7.28-7.32 (m, 2H); 7.38-7.42 (m, 3H); 7.47-7.50 (m, 2H); 7.99 (d, 1H); 8.06 (d, 2H).

Rn-azide-Ni—R—BPB (R=H), 13. To Gly-Ni—R—BPB (10.0 mmol) and KO-tBu (1.5 eq.) was added 45 mL of DMF under argon. The compound 1 (1.5 eq.) in solution of DMF (4.0 mL) was added via syringe. The reaction mixture was stirred at ambient temperature for 1 h. The solution was then quenched with 5% aqueous acetic acid and diluted with water. The oily product was collected by filtration and washed with water. The desired product 13 was purified by flash chromatography on normal phase using acetone and dichloromethane as eluents to give a red solid in 55% yield. R5-azide-Ni—R—BPB (13, R=H, n=5): M+H calc. 609.20. M+H obs. 609.18; δ: 1.18 (m, 2H, CH₂); 1.52 (m, 4H, CH₂); 2.06 (m, 3H, CH₂); 2.17 (m, 1H, CH₂); 2.53 (m, 1H, CH₂); 2.74 (m, 1H, CH₂); 3.20 (m, 2H, CH₂); 3.48 (m, 2H, CH₂); 3.55 (m, 1H, CH_{α}); 3.90 (m, 1H, CH_{α}′); 3.58 and 4.44 (AB system, 2H, CH₂ benzyl); 6.63 (m, 2H); 6.92 (d, 1H); 7.11-7.21 (m, 2H); 7.27 (m, 1H); 7.32-7.36 (m, 2H); 7.46-7.50 (m, 3H); 8.04 (d, 2H); 8.11 (d, 1H). R6-azide-Ni—R—BPB (13, R=H, n=6): M+H calc. 623.22. M+H obs. 623.19; ¹H NMR (CDCl₃) δ: 1.16 (m, 2H, CH₂); 1.32 (m, 2H, CH₂); 1.54 (m, 4H, CH₂); 2.05 (m, 3H, CH₂); 2.16 (m, 1H, CH₂); 2.53 (m, 1H, CH₂); 2.74 (m, 1H, CH₂); 3.22 (m, 2H, CH₂); 3.48 (m, 2H, CH₂); 3.58 (m, 1H, CH_{α}); 3.90 (m, 1H, CH_{α}′); 3.59 and 4.44 (AB system, 2H, CH₂ benzyl); 6.63 (m, 2H); 6.92 (d, 1H); 7.11-7.21 (m, 2H); 7.27 (m, 1H); 7.32-7.36 (m, 2H); 7.45 (m, 1H); 7.50 (m, 2H); 8.04 (d, 2H); 8.11 (d, 1H).

Fmoc-αMe-Rn-azide-OH (R=Me), 14. To a solution of 3N HCl/MeOH (1/1, 12 mL) at 70° C. was added a solution of compound 13, R=Me (1.65 mmol) in MeOH (3 ml) dropwise. The starting material disappeared within 10-20 min. The green reaction mixture was then concentrated in vacuo. The crude residue was diluted with 10% aqueous Na₂CO₃ (16 ml) and cooled to 0° C. with an ice bath. Fmoc-OSu (1.1 eq.) dissolved in acetone (16 ml) was added and the reaction was allowed to warm up to ambient temperature with stirring overnight. Afterwards, the reaction was diluted with ethyl acetate and 1 N HCl. The organic layer was washed with 1 N HCl (3×). The organic layer was then dried over magnesium sulfate and concentrated in vacuo. The desired product 14 was purified on normal phase using methanol and dichloromethane as eluents to give a viscous oil in 36% overall yield for both steps. Fmoc-αMe-R5-azide-OH (14, R=Me, n=5): M+H calc. 423.20. M+H obs. 423.34; ¹H NMR (CDCl₃) δ: 0.90 (bs, 2H, CH₂); 1.36 (bs, 2H, CH₂); 1.56 (m, 2H); 1.60 (bs, 3H, Me (αMe-R5-azide)); 1.86 (bs, 1H, CH₂); 2.15 (bs, 1H, CH₂); 3.23 (bs, 2H, CH₂); 4.22 (m, 1H, CH Fmoc); 4.40 (bs, 2H, CH₂ Fmoc); 5.51 (bs, 1H, NH); 7.32 (m, 2H); 7.40 (m, 2H); 7.59 (d, 2H); 7.78 (d, 2H). Fmoc-αMe-R6-azide-OH (14, R=Me, n=6): M+H calc. 437.21. M+H obs. 437.31; ¹H NMR (CDCl₃) δ: 0.90 (bs, 2H, CH₂); 1.32 (bs, 4H, CH₂); 1.56 (m, 2H); 1.61 (bs, 3H, Me (αMe-R6-azide)); 1.84 (bs, 1H, CH₂); 2.13 (bs, 1H, CH₂); 3.23 (t, 2H, CH₂); 4.22 (m, 1H, CH Fmoc); 4.39 (bs, 2H, CH₂ Fmoc); 5.51 (bs, 1H, NH); 7.32 (m, 2H); 7.40 (m, 2H); 7.59 (d, 2H); 7.77 (d, 2H).

Fmoc-Rn-azide-OH (R=H), 14. To a solution of 3N HCl/MeOH (1/1, 12 mL) at 70° C. was added a solution of compound 13, R=H (1.65 mmol) in MeOH (3 ml) dropwise. The starting material disappeared within 10-20 min. The green reaction mixture was then concentrated in vacuo. The crude residue was diluted with 10% aqueous Na₂CO₃ (16 ml) and cooled to 0° C. with an ice bath. Fmoc-OSu (1.1 eq.) dissolved in acetone (16 ml) was added and the reaction was allowed to warm up to ambient temperature with stirring overnight. Afterwards, the reaction was diluted with ethyl acetate and 1 N HCl. The organic layer was washed with 1 N HCl (3×). The organic layer was then dried over magnesium sulfate and concentrated in vacuo. The desired product 14 was purified on normal phase using methanol and dichloromethane as eluents to give a viscous oil in 36% overall yield for both steps. Fmoc-R5-azide-OH (14, R=H, n=5): M+H calc. 409.18. M+H obs. 409.37; ¹H NMR (CDCl₃) δ: 1.29 (bs, 2H, CH₂); 1.40 (bs, 2H, CH₂); 1.60 (m, 2H); 1.72 (bs, 1H, CH₂); 1.90 (bs, 1H, CH₂); 3.26 (m, 2H, CH₂); 4.23 (m, 1H, CH Fmoc); 4.41 (m, 3H, CH₂ Fmoc+CH_{α}); 5.30 (d, 1H, NH); 7.32 (m, 2H); 7.40 (m, 2H); 7.59 (d, 2H); 7.78 (d, 2H). Fmoc-R6-azide-OH (14, R=H, n=6): M+H calc. 423.20, M+H obs. 423.34; NMR (CDCl₃) δ: 1.37 (bs, 6H, CH₂); 1.59 (bs, 2H, CH₂); 1.70 (bs, 1H, CH₂); 1.90 (bs, 1H, CH₂); 3.25 (m, 2H, CH₂); 4.23 (m, 1H, CH Fmoc); 4.41 (m, 3H, CH₂ Fmoc+CH_{α}); 5.24 (d, 1H, NH); 7.32 (m, 2H); 7.39 (m, 2H); 7.59 (m, 2H); 7.76 (d, 2H).

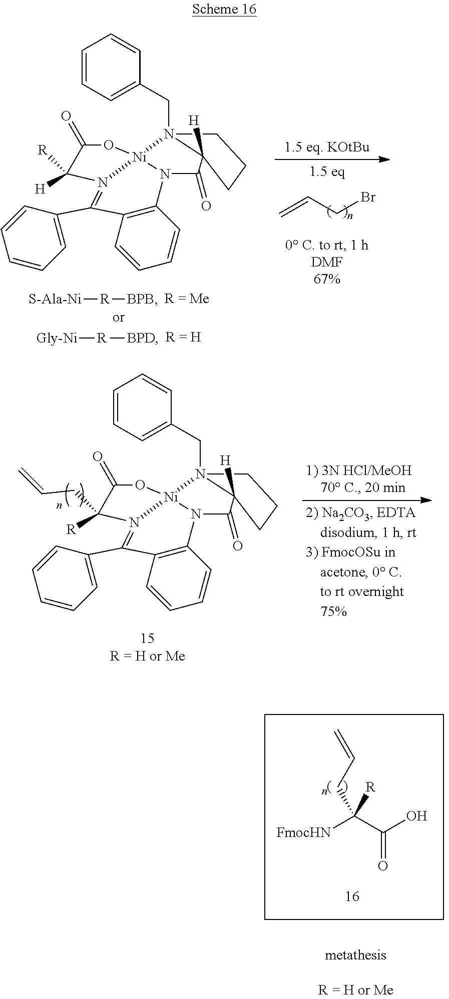

αMe-R(n+2)-alkene-Ni—R—BPB (R=Me), 15. To R-Ala-Ni—R—BPB (10.0 mmol) and KO-tBu (2 eq.) was added 45 mL of DMF under argon. 1-Bromo-n-alkene (1.5 eq.) in solution of DMF (4.0 mL) was added via syringe. The reaction was stirred at ambient temperature for 1 h. The reaction was then quenched with 5% aqueous acetic acid and diluted with water. The oily product was collected by filtration and washed with water. The desired product 15 was purified by flash chromatography on normal phase using acetone and dichloromethane as eluents to give a red solid in 55% yield. αMe-R8-alkene-Ni—R—BPB (7, R=Me, n=6): M+H calc. 622.25. M+H obs. 622.22; ¹H NMR (CDCl₃) δ: 1.24 (s, 3H, Me (αMe-S8-alkene)); 1.29-1.44 (m, 5H, CH₂); 1.56-1.74 (m, 3H, CH₂); 2.06 (m, 5H, CH₂); 2.32-2.51 (m, 2H, CH₂); 2.68 (m, 1H, CH₂); 3.28 (m, 1H, CH₂); 3.42 (m, 1H, CH₂); 3.62 (m, 1H, CH_{α}); 3.70 and 4.50 (AB system, 2H, CH₂ (benzyl), J=12.8 Hz); 4.92-5.02 (m, 2H, CH₂ alkene); 5.76-5.85 (m, 1H, CH alkene); 6.63 (m, 2H); 6.96 (d, 1H); 7.12 (m, 1H); 7.27-7.33 (m, 2H); 7.38-7.42 (m, 3H); 7.45-7.51 (m, 2H); 7.98 (d, 1H); 8.06 (d, 2H).

R(n+2)-alkene-Ni—R—BPB (R=H), 15. To Gly-Ni—R—BPB (10.0 mmol) and KO-tBu (2 eq.) was added 45 mL of DMF under argon. 1-Bromo-n-alkene (1.5 eq.) in solution of DMF (4.0 mL) was added via syringe. The reaction was stirred at ambient temperature for 1 h. The reaction was then quenched with 5% aqueous acetic acid and diluted with water. The oily product was collected by filtration and washed with water. The desired product 15 was purified by flash chromatography on normal phase using acetone and dichloromethane as eluents to give a red solid in 55% yield. R8-alkene-Ni—R—BPB (15, R=H, n=6): M+H calc. 608.23. M+H obs. 608.21; ¹H NMR (CDCl₃) δ: 1.14 (m, 2H, CH₂); 1.30 (m, 4H, CH₂); 1.61 (m, 2H, CH₂); 1.92-2.16 (m, 6H, CH₂); 2.52 (m, 1H, CH₂); 2.75 (m, 1H, CH₂); 3.44-3.52 (m, 2H, CH₂); 3.58 (m, 1H, CH_{α}); 3.91 (m, 1H, CHO; 3.58 and 4.44 (AB system, 2H, CH₂ (benzyl)); 4.92-5.00 (m, 2H, CH₂ alkene); 5.78 (m, 1H, CH alkene); 6.63 (m, 2H); 6.91 (d, 1H); 7.13-7.18 (m, 2H); 7.24 (m, 1H); 7.34 (m, 2H); 7.38-7.49 (m, 3H); 8.03 (d, 2H); 8.12 (d, 1H).

Fmoc-αMe-R(n+2)-alkene-OH (R=Me), 16. To a solution (18 mL) of 1/1 3N HCl/MeOH at 70° C. was added a solution of compound 15, R=Me (2.4 mmol) in MeOH (4 ml) dropwise. The starting material disappeared within 5-10 min. The green solution was then concentrated in vacuo. The crude residue was diluted with 10% aqueous Na₂CO₃ (24 ml) cooled to 0° C. with an ice bath. Fmoc-OSu (1.1 eq.) dissolved in acetone (24 ml) was added and the reaction was allowed to warm up to ambient temperature with stirring overnight. Afterwards, the reaction was diluted with ethyl acetate and 1 N HCl. The organic layer was washed with 1 N HCl (3×). The organic layer was then dried over magnesium sulfate and concentrated in vacuo. The desired product 16 was isolated after flash chromatography purification on normal phase using methanol and dichloromethane as eluents to give viscous oil that solidifies upon standing in 75% yield. Fmoc-αMe-R8-alkene-OH (16, R=Me, n=6): M+H calc. 422.23. M+H obs. 422.22; ¹H NMR (CDCl₃) δ: 1.28 (m, 8H, CH₂); 1.60 (s, 3H, αMe); 1.83 (m, 1H, CH₂); 2.01 (m, 2H, CH₂); 2.11 (m, 1H, CH₂); 4.22 (m, 1H, CH (Fmoc)); 4.39 (m, 2H, CH₂ (Fmoc)); 4.90-5.00 (m, 2H, CH₂ alkene); 5.49 (bs, 1H, NH); 5.75-5.82 (m, 1H, CH alkene); 7.29-7.33 (m, 2H); 7.38-7.42 (m, 2H); 7.59 (d, 2H); 7.77 (d, 2H).

Fmoc-R(n+2)-alkene-OH (R=H), 16. To a solution (18 mL) of 1/1 3N HCl/MeOH at 70° C. was added a solution of compound 15, R=H (2.4 mmol) in MeOH (4 ml) dropwise. The starting material disappeared within 5-10 min. The green solution was then concentrated in vacuo. The crude residue was diluted with 10% aqueous Na₂CO₃ (24 ml) cooled to 0° C. with an ice bath. Fmoc-OSu (1.1 eq.) dissolved in acetone (24 ml) was added and the reaction was allowed to warm up to ambient temperature with stirring overnight. Afterwards, the reaction was diluted with ethyl acetate and 1 N HCl. The organic layer was washed with 1 N HCl (3×). The organic layer was then dried over magnesium sulfate and concentrated in vacuo. The desired product 16 was isolated after flash chromatography purification on normal phase using methanol and dichloromethane as eluents to give viscous oil that solidifies upon standing in 75% yield. Fmoc-R8-alkene-OH (16, R=H, n=6): M+H calc. 407.21. M+H obs. 407.19; ¹H NMR (CDCl₃) δ: 1.32 (m, 8H, CH₂); 1.71 (m, 1H); 1.89 (m, 1H, CH₂); 2.03 (m, 2H); 4.23 (m, 1H, CH (Fmoc)); 4.42 (m, 2H, CH₂ (Fmoc)); 4.96 (m, 2H, CH₂ alkene+CH_{α}); 5.20 (d, 1H, NH); 5.79 (m, 1H, CH alkene); 7.32 (m, 2H); 7.41 (m, 2H); 7.59 (m, 2H); 7.77 (d, 2H).

The non-natural amino acids (R and S enantiomers of the 5-carbon olefinic amino acid and the S enantiomer of the 8-carbon olefinic amino acid) were characterized by nuclear magnetic resonance (NMR) spectroscopy (Varian Mercury 400) and mass spectrometry (Micromass LCT). Peptide synthesis was performed either manually or on an automated peptide synthesizer (Applied Biosystems, model 433A), using solid phase conditions, rink amide AM resin (Novabiochem), and Fmoc main-chain protecting group chemistry. For the coupling of natural Fmoc-protected amino acids (Novabiochem), 10 equivalents of amino acid and a 1:1:2 molar ratio of coupling reagents HBTU/HOBt (Novabiochem)/DIEA were employed. Non-natural amino acids (4 equiv) were coupled with a 1:1:2 molar ratio of HATU (Applied Biosystems)/HOBt/DIEA, or as further described below. Olefin metathesis was performed in the solid phase using 10 mM Grubbs catalyst (Blackewell et al. 1994 supra) (Strem Chemicals) dissolved in degassed dichloromethane and reacted for 2 hours at room temperature. Isolation of metathesized compounds was achieved by trifluoroacetic acid-mediated deprotection and cleavage, ether precipitation to yield the crude product, and high performance liquid chromatography (HPLC) (Varian ProStar) on a reverse phase C18 column (Varian) to yield the pure compounds. Chemical composition of the pure products was confirmed by LC/MS mass spectrometry (Micromass LCT interfaced with Agilent 1100 HPLC system) and amino acid analysis (Applied Biosystems, model 420A).

### Example 2

### Synthesis of Peptidomimetic Macrocycles of the Invention

α-helical BID peptidomimetic macrocycles were synthesized, purified and analyzed as previously described (Walensky et al (2004) Science 305:1466-70; Walensky et al (2006) Mol Cell 24:199-210) and as indicated below. The following macrocycles were used in this study:

| | | SEQ | | Calculated | Calculated | Found |
|---|---|---|---|---|---|---|
| Macro- | WT | ID | | m/z | m/z | m/z |
| cycle | Sequence | NO: | Sequence | (M + H) | (M + 3H) | (M + 3H) |
| SP-4 | BIM-BH3 | 115 | Ac-IWIAQELR$IGD$FNAYYARR-NH2 | 2646.4306 | 882.8154 | 883.15 |
| SP-54 | BIM-BH3 | 116 | Ac-IWIAQELR#IGD#FNAYYARR-NH2 | 2618.3993 | 873.4716 | 873.39 |
| SP-27 | BIM-BH3 | 117 | Ac-IWIAQELR#sIGD#sFNAYYARR-NH2 | 2622.3578 | 874.7911 | 875.17 |
| | BIM-BH3 | 117 | Ac-IWIAQELR#sIGD#sFNAYYARR-NH2 | 2622.3578 | 874.7911 | 875.10 |
| SP-28 | BIM-BH3 | 118 | Ac-IWIAQELR$sIGD$sFNAYYARR-NH2 | 2650.3891 | 884.1349 | 883.97 |
| | BIM-BH3 | 118 | Ac-IWIAQELR$sIGD$sFNAYYARR-NH2 | 2650.3891 | 884.1349 | 884.04 |
| SP-29 | BIM-BH3 | 119 | Ac-IWIAQELR#c4IGD#c4FNAYYARR-NH2 | 2656.3278 | 886.1145 | 886.48 |
| SP-30 | BIM-BH3 | 120 | Ac-IWIAQELR$c4IGD$c4FNAYYARR-NH2 | 2684.3591 | 895.4582 | 895.81 |
| SP-31 | BIM-BH3 | 121 | Ac-IWIAQELR#5n3IGD#5a5FNAYYARR-NH2 | 2659.4007 | 887.1388 | 887.01 |
| | BIM-BH3 | 121 | Ac-IWIAQELR#5n3IGD#5a5FNAYYARR-NH2 | 2659.4007 | 887.1388 | 887.21 |
| SP-32 | BIM-BH3 | 122 | Ac-IWIAQELR$5n3IGD$5a5FNAYYARR-NH2 | 2687.4320 | 896.4825 | 896.74 |
| SP-33 | BID-BH3 | 123 | Ac-DIIRNIARHLA#c4VGD#c4NleDRSI-NH2 | 2448.2965 | 816.7707 | 817.07 |
| SP-34 | BID-BH3 | 124 | Ac-DIIRNIARHLA$c4VGD$c4NleDRSI-NH2 | 2476.3278 | 826.1145 | 826.40 |
| SP-1 | BID-BH3 | 125 | Ac-DIIRNIARHLA$VGD$NleDRSI-NH2 | 2438.3993 | 813.4716 | 813.76 |
| SP-35 | BID-BH3 | 126 | Ac-DIIRNIARHLA#VGD#NleDRSI-NH2 | 2410.3680 | 804.1279 | 804.50 |
| SP-36 | BID-BH3 | 127 | Ac-DIIRNIARHLA#cVGD#cNleDRSI-NH2 | 2446.2808 | 816.0988 | 816.41 |
| | BID-BH3 | 127 | Ac-DIIRNIARHLA#cVGD#cNleDRSI-NH2 | 2446.2808 | 816.0988 | 816.34 |
| SP-37 | BID-BH3 | 128 | Ac-DIIRNIARHLA$cVGD$cNleDRSI-NH2 | 2474.3121 | 825.4426 | 825.61 |
| | BID-BH3 | 128 | Ac-DIIRNIARHLA$cVGD$cNleDRSI-NH2 | 2474.3121 | 825.4426 | 825.74 |
| SP-38 | BID-BH3 | 129 | Ac-DIIRNIARHLA#sVGD#sNleDRSI-NH2 | 2414.3265 | 805.4474 | 805.82 |
| | BID-BH3 | 129 | Ac-DIIRNIARHLA#sVGD#sNleDRSI-NH2 | 2414.3265 | 805.4474 | 805.82 |
| SP-39 | BID-BH3 | 130 | Ac-DIIRNIARHLA$sVGD$sNleDRSI-NH2 | 2442.3578 | 814.7911 | 815.15 |
| | BID-BH3 | 130 | Ac-DIIRNIARHLA$sVGD$sNleDRSI-NH2 | 2442.3578 | 814.7911 | 815.09 |
| SP-40 | BIM-BH3 | 131 | Ac-IWIAQELR#cIGD#cFNAYYARR-NH2 | 2654.3121 | 885.4426 | 885.76 |
| | BIM-BH3 | 131 | Ac-IWIAQELR#cIGD#cFNAYYARR-NH2 | 2654.3121 | 885.4426 | 885.42 |
| SP-41 | BIM-BH3 | 132 | Ac-IWIAQELR$cIGD$cFNAYYARR-NH2 | 2682.3434 | 894.7863 | 895.15 |
| SP-42 | p53 | 133 | 5-QSQQTF#r8NLWRLL#QN-NH2 | 2081.1294 | 694.3817 | 1041.38* |
| SP-43 | p53 | 134 | 5-QSQQTF$r8NLWRLL$QN-NH2 | 2109.1607 | 703.7254 | 1054.98* |
| SP-44 | p53 | 135 | 5-QSQQTF$5rn6NLWRLL$5a5QN-NH2 | 2150.1621 | 717.3926 | 1075.91* |
| SP-45 | p53 | 136 | 5-QSQQTF#5rn6NLWRLL#5a5QN-NH2 | 2122.1308 | 708.0488 | 1062.02* |
| SP-46 | p53 | 137 | 5-QSQQTF$4rn5NLWRLL$4a5QN-NH2 | 2136.1464 | 712.7207 | 1069.03* |
| SP-47 | p53 | 138 | 5-QSQQTF#4rn5NLWRLL#4a5QN-NH2 | 2108.1151 | 703.3769 | 1055.00* |
| SP-48 | BIM-BH3 | 139 | FITC-Ahx-IWIAQELR$5n3IGD$5a5FNAYYARR-NH2 | 3149.5569 | 1050.5242 | 1050.44 |
| SP-49 | BIM-BH3 | 140 | FITC-Ahx-IWIAQELR#5n3IGD#5a5FNAYYARR-NH2 | 3121.5256 | 1041.1804 | 1041.04 |
| | BIM-BH3 | 140 | FITC-Ahx-IWIAQELR#5n3IGD#5a5FNAYYARR-NH2 | 3121.5256 | 1041.1804 | 1040.78 |
| SP-50 | p53 | 141 | 5-FAM-QSQQTF$5rn6NLWRLL$5a5QN-NH2 | 2466.1992 | 822.7383 | 823.03 |
| SP-51 | p53 | 142 | 5-FAM-QSQQTF#5rn6NLWRLL#5a5QN-NH2 | 2438.1679 | 813.3945 | 813.70 |
| SP-52 | BID-BH3 | 143 | Ac-DIIRNIARHLA%VGD%NleDRSI-NH2 | | | |
| SP-53 | BID-BH3 | 144 | Ac-DIIRNIARHLA%VAibD%NleDRSI-NH2 | | | |

*= M + 2H

Alpha,alpha-disubstituted non-natural amino acids containing olefinic side chains were synthesized according to Williams et al. (1991) J. Am. Chem. Soc. 113:9276; and Schafmeister et al. (2000) J. Am. Chem Soc. 122:5891. Peptidomimetic macrocycles were designed by replacing two naturally occurring amino acids (see above) with the corresponding synthetic amino acids. Substitutions were made at the i and i+4 and i to i+7 positions as indicated. Peptidomimetic macrocycles were generated by solid phase peptide synthesis followed by crosslinking of the synthetic amino acids via the reactive moieties of their side chains. The control sequences for BID and BIM peptidomimetic macrocycles are shown above. In the above table, where two sequences are indicated for a single macrocycle name, each sequence represents an isomer obtained as a result of the crosslinking reaction.

In the above sequences, the following nomenclature is used:
$ Alpha-Me S5 olefin amino acid
# Alpha-H S5 olefin amino acid
$r8 Alpha-Me R8 olefin amino acid
#r8 Alpha-H R8 olefin amino acid
$s Alpha-Me O-allyl serine
#s Alpha-H O-allyl serine
$c Alpha-Me S-allyl cysteine
#c Alpha-H S-allyl cysteine
$c4 Alpha-Me cysteine butyl thioether
#c4 Alpha-H cysteine butyl thioether
$5n3 Alpha-Me azide 1,5 triazole (3 carbon)
#5n3 Alpha-H azide 1,5 triazole (3 carbon)
$5a5 Alpha-Me alkyne 1,5 triazole (5 carbon)
#5a5 Alpha-H alkyne 1,5 triazole (5 carbon)
$5rn6 Alpha-Me R-azide 1,5 triazole (6 carbon)
#5rn6 Alpha-H R-azide 1,5 triazole (6 carbon)
$4rn5 Alpha-Me R-azide 1,4 triazole (5 carbon)
#4rn5 Alpha-H R-azide 1,4 triazole (5 carbon)
Ahx aminohexyl (linker)

In the sequences above, Nle represents norleucine and Aib represents 2-aminoisobutyric acid.

Amino acids represented as % connect an all-carbon crosslinker comprising only single bonds and wherein each α-carbon atom to which the crosslinker is attached is additionally substituted with a methyl group. Such a crosslink is prepared using olefin metathesis of precursors containing alpha-methyl S5 olefin amino acids, followed by reduction of the crosslink.

The following structural drawings further illustrate a number of crosslinks in peptidomimetic macrocycles of the invention.

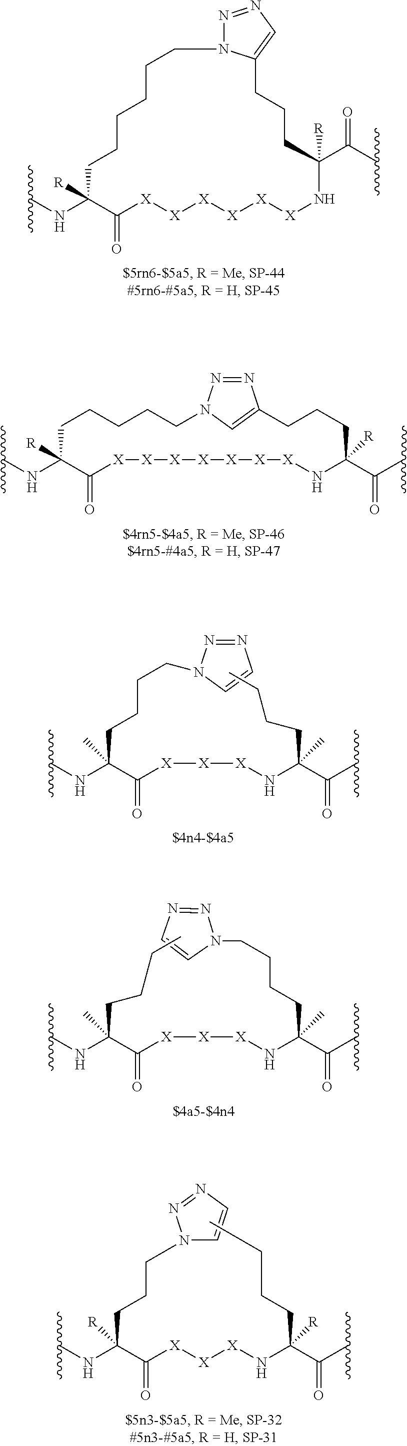

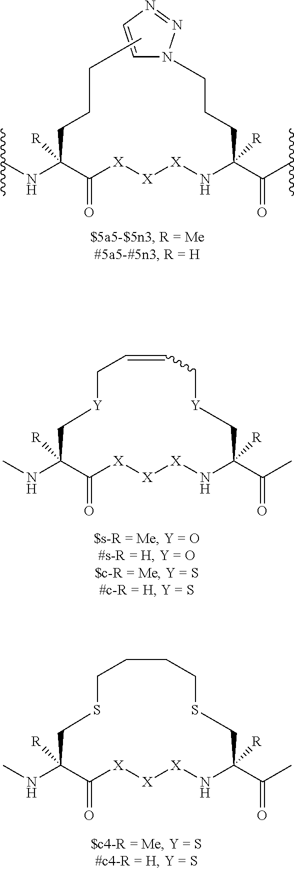

### Example 3

### Intramolecular (i to i+4 and i to i+7) Side-Chain to Side-Chain Azide-Alkyne Huisgen 1,3-Dipolar Cycloaddition on Peptide Bound on Resin

The fully protected resin-bound peptides were synthesized on a Rink amide MBHA resin (loading 0.62 mmol/g) on a 0.2 mmol scale. Deprotection of the temporary Fmoc group was achieved by 2×20 min treatments of the resin bound peptide with 25% (v/v) piperidine in NMP. After extensive flow washing with NMP, methanol and dichloromethane, coupling of each successive amino acid was achieved with 1×60 min incubation with the appropriate preactivated Fmoc-amino acid derivative. All protected amino acids (1 mmol) were dissolved in NMP and activated with HCTU (1 mmol) and DIEA (1 mmol) prior to transfer of the coupling solution to the deprotected resin-bound peptide. After coupling was completed, the resin was extensively flow washed in preparation for the next deprotection/coupling cycle. Acetylation of the amino terminus was carried out in the presence of acetic anhydride/DIEA in NMP/NMM. The LC-MS analysis of a cleaved and deprotected sample obtained from an aliquote of the fully assembled resin-bound peptide was accomplished in order to verify the completion of each coupling. For copper-catalyzed azide-alkyne cycloaddition, the azide/acetylene-containing peptide bound on resin (Rink amide MBHA, loading 0.62 mmol/g) was subjected to the 1,4-triazole formation using CuI (5 equiv), DIPEA (10 equiv), sodium L-ascorbate (5 equiv) in 10 ml of 30% 2,6-lutidine in DMF. The reaction mixture was shaken gently. The reaction was allowed to proceed overnight at room temperature. For ruthenium-catalyzed azide-alkyne cycloaddition, the azide/acetylene-containing peptide bound on resin (Rink amide MBHA, loading 0.62 mmol/g) was subjected to the 1,5-triazole formation using Cp*RuCl(PPh₃)₂ (10 mol %) in 10 ml of benzene. The reaction mixture was shaken gently. The reaction was allowed to proceed overnight at 80° C. This procedure was repeated once for completion of the cycloaddition.

Following the coupling reaction, the triazole-containing resin-bound peptides were deprotected and cleaved from the solid support by treatment with TFA/H₂O/TIS (94/3/3 v/v) for 3 h at room temperature. After filtration of the resin the TFA solution was precipitated in cold diethyl ether and centrifuged to yield the desired product as a solid. The crude product was purified by preparative HPLC.

In the case of SP-31 (R=H) macrocycles, the above procedures resulted in two isomers corresponding to 1,4- and 1,5-triazole crosslink configurations. However, only one isomer was observed for SP-32 (R=Me) macrocycles.

### Example 4

### Synthesis of Additional Peptidomimetic Macrocycles of the Invention

Peptidomimetic macrocycles were elongated on a Thuramed Tetras automated multichannel peptide synthesizer starting with a 4-(2′4′-dimethoxyphenyl-Fmoc-aminomethyl)-phenoxyacetamido-norleucylaminomethyl linked polystyrene resin (Rink AM resin). The amino acids (10 eq) were coupled using standard solid phase protocols based on fluorenylmethoxycarbonyl (Fmoc) protection and 2-(6-Chloro-1H-benzotriazole-1-yl)-1,1,3,3-tetramethylaminium hexafluorophosphate (HCTU) as the coupling agent (10 eq). Double coupling was used during the automated process for all of the amino acids except for the α-methylated Fmoc-protected olefinic amino acids which were single coupled with longer reaction times. After the final amino acid was added to the peptide, the Fmoc group was removed and the free amine was acylated using acetic anhydride in 10% DIEA in DMF.

SP-33 (R=H): The linear peptide (assembled as above) on resin (0.3 mmol based on initial resin loading) was simultaneously cleaved and the protecting groups on the sidechains removed by treating the resin with a solution (20 mL) of trifluoroacetic acid (TFA) (93.5%), water (2.5%), triisopropylsilane (TIPS), (2.5%), and ethanedithiol (EDT) (2.5%). The mixture was filtered and to the filtrate was added chilled diethylether (100 mL). The mixture was centrifuged and the supernatant decanted. The pellet was suspended in 1:1 acetonitrile/water (5 mL) and lyophilized. The crude linear peptide was purified using C₁₈ reversed-phase HPLC with acetonitrile and water (with 0.1% TFA) as the mobile phase. The fractions containing the desired peptide were pooled and lyophilized to give the linear peptide as a colorless solid (65 mg). To the linear peptide (45 mg, 18 μmol) was added anhydrous MeOH (8 mL). Condensed liquid ammonia (60 mL) was added to the peptide solution followed by 1,4-dibromobutane (36 μL of 10% solution in MeOH, 29 μmol). The reaction was allowed to reflux and was slowly allowed to warm to room temperature. The remaining methanol was removed under reduced pressure. The crude linear peptide was purified using C₁₈ reversed-phase HPLC with acetonitrile and (with 0.1% TFA) as the mobile phase. The fractions containing the desired peptide were pooled and lyophilized to give the SP-33 as a colorless solid (11.2 mg). MS (ESI) m/z, found 817.07 (M+3H/3). calcd. 816.77 (M+3H/3).

SP-34 (R=—CH₃): The α-methylated cysteine was synthesized using published procedures (Seebach et al. (1996), Angew. Chem. Int. Ed. Engl. 35:2708-2748, and references therein) and then converted to the appropriately protected N-α-Fmoc-S-trityl monomers by known methods (“Bioorganic Chemistry: Peptides and Proteins”, Oxford University Press, New York: 1998, the entire contents of which are incorporated herein by reference). The peptide is synthesized in the same manner as SP-33 to yield SP-34 as a colorless solid (6.1 mg). MS (ESI) m/z, found 817.07 (M+3H/3). calcd. 826.11 (M+3H/3).

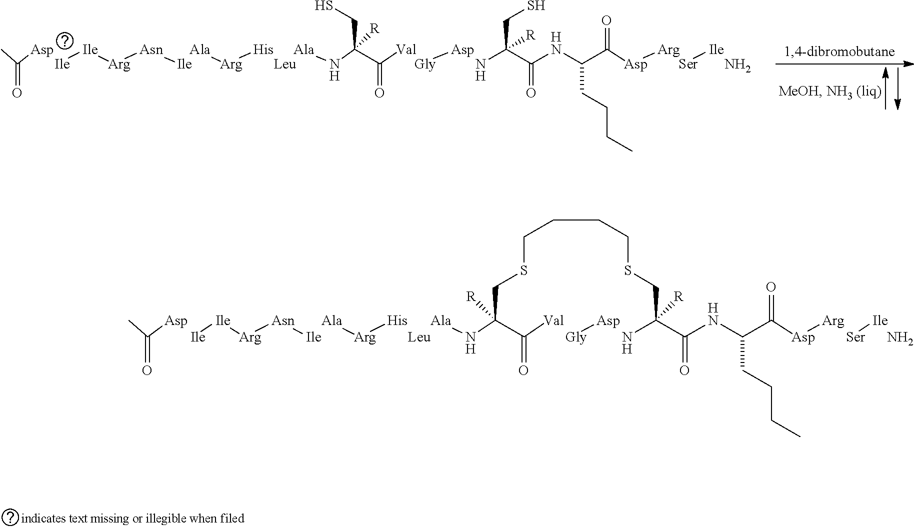

(SEQ ID NOS 145 and 124, respectively)

SP-29 (R=—H): The peptide was synthesized in the same manner as SP-33 to yield SP-29 as a colorless solid (7.1 mg). MS (ESI) m/z, found 886.75 (M+3H/3). calcd. 886.11 (M+3H/3).

SP-30 (R=—CH₃): The α-methylated cysteine was synthesized using published procedures (Seebach et al. (1996), Angew. Chem. Int. Ed. Engl. 35:2708-2748, and references therein) and then converted to the appropriately protected N-α-Fmoc-S-trityl monomers by known methods (“Bioorganic Chemistry: Peptides and Proteins”, Oxford University Press, New York: 1998, the entire contents of which are incorporated herein by reference). The peptide was synthesized in the same manner as SP-33 to yield SP-30 as a colorless solid (4.1 mg). MS (ESI) m/z, found 896.08 (M+3H/3). calcd. 895.46 (M+3H/3).

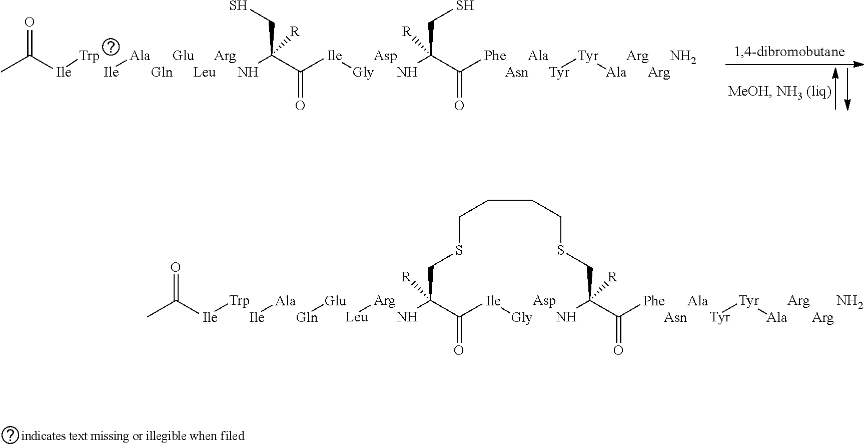

(SEQ ID NOS 146 and 120, respectively)

SP-41 (R=CH₃): The α-methylated cysteine was synthesized using published procedures (Seebach et al. (1996), Angew. Chem. Int. Ed. Engl. 35:2708-2748, and references therein) and then converted to the appropriately protected N-α-Fmoc-S-trityl monomers by known methods (“Bioorganic Chemistry: Peptides and Proteins”, Oxford University Press, New York: 1998, the entire contents of which are incorporated herein by reference). The linear peptide (assembled as above) on resin (0.1 mmol based on initial resin loading) was treated with TFA (1%), TIPS (4%) in DCM (3 min, 10 cycles) to selectively remove the Mmt-protected thiols. The resin was washed successively with DCM and 10% DIEA/NMP. The resin was suspended in anhydrous DMF (1 mL) and DIEA (87 μL, 0.5 mmol) Allyl bromide (22 μL, 0.25 mmol) was added to the mixture and the reaction was agitated at room temperature. After 1 h, the reaction was filtered and the resin was washed successively with DMF, DCM and diethyl ether. The resin was dried under reduced pressure and taken up in an anhydrous DCM solution of Grubbs I catalyst (4 mL, 4 mg/mL, 0.02 mmol) After 18 h, the reaction was filtered and the resin was washed with DCM. The olefin metathesis step was repeated twice in order to fully consume starting material. The resin was taken up in 10% EDT/DMF (4 mL) and agitated at ambient temperature for 18 h. The resin was filtered and washed successively with NMP, DCM and ether. The cyclized peptide was simultaneously cleaved from the resin and the protecting groups on the sidechains removed by treating the resin with a solution (7.5 mL) of trifluoroacetic acid (TFA) (93.5%), water (2.5%), triisopropylsilane (TIPS), (2.5%), and ethanedithiol (EDT) (2.5%). The mixture was filtered and to the filtrate was added chilled diethylether (40 mL). The mixture was centrifuged and the supernatant decanted. The pellet was suspended in 1:1 acetonitrile/water (5 mL) and lyophilized. The crude peptide was purified using C₁₈ reversed-phase HPLC with acetonitrile and water (with 0.1% TFA) as the mobile phase. The fractions containing the desired peptide were pooled. The fractions were lyophilized twice in 50:50 acetonitrile: HCl (aq) (60 mN, then 10 mN) and once in 50:50 acetonitrile:water to give SP-41 as a colorless solid (5.9 mg). MS (ESI) m/z, found 895.42 (M+3H/3). calcd. 894.79 (M+3H/3).

SP-40 (R=—H): The peptide was synthesized in the same manner as SP-41 to yield two isomers of SP-40 as a colorless solids; ealier eluting isomer (9.7 mg), later eluting isomer (13.3 mg). MS (ESI) m/z, found 886.02 (M+3H/3). calcd. 885.44 (M+3H/3).

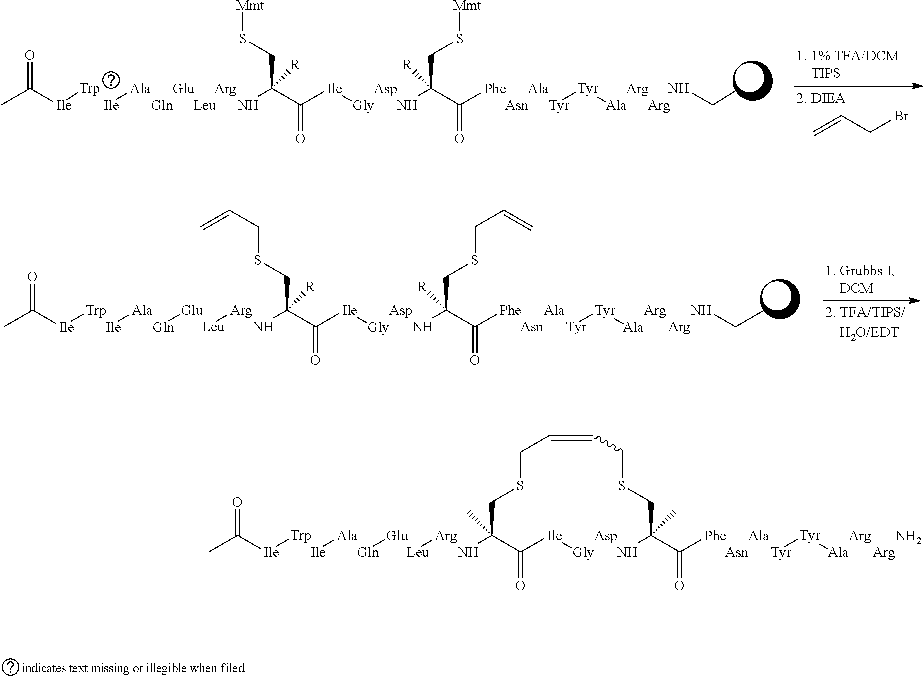

(SEQ ID NOS 147-148 and 131, respectively)

SP-36 (R=—H): The peptide was synthesized in the same manner as SP-41 to yield two isomers of SP-36 as a colorless solids; ealier eluting isomer (9.5 mg), later eluting isomer (10.2 mg) MS (ESI) m/z, found 816.74 (M+3H/3). calcd. 816.10 (M+3H/3).

SP-37 (R=—CH₃): The peptide was synthesized in the same manner as SP-41 to yield two isomers of SP-37 as a colorless solid; ealier eluting isomer (1.7 mg), later eluting isomer (1.6 mg). MS (ESI) m/z, found 825.74 (M+3H/3). calcd. 825.44 (M+3H/3).

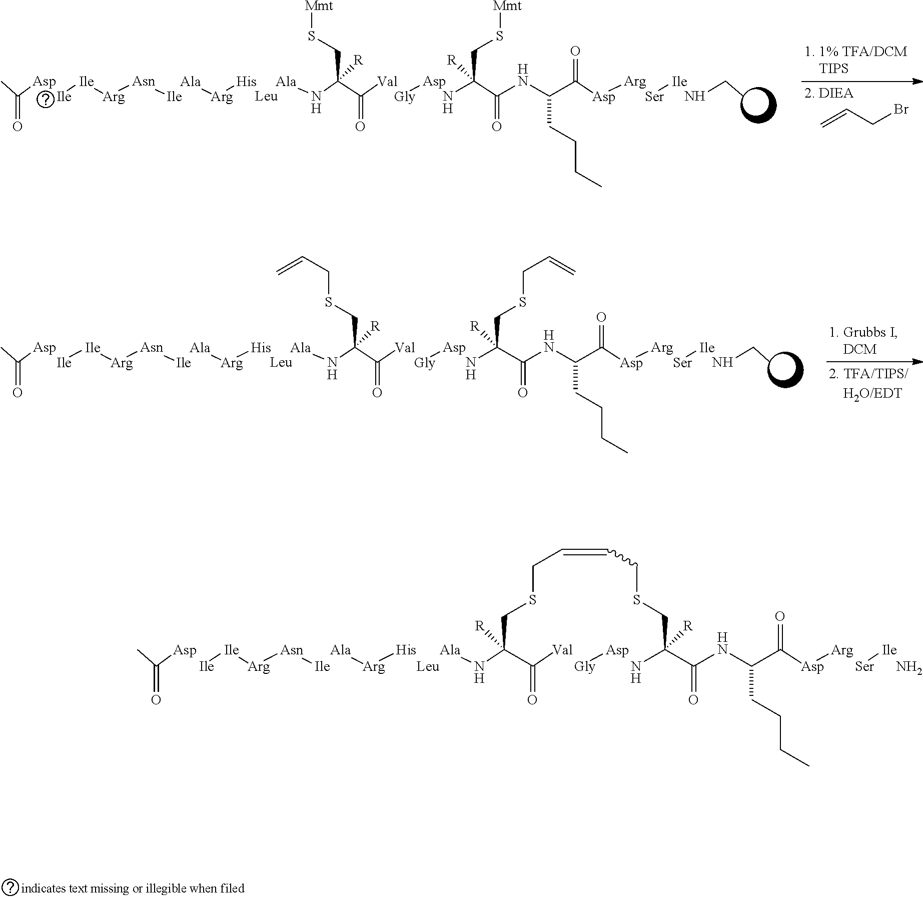

(SEQ ID NOS 149-150 and 128, respectively)

SP-27 (R=H): The linear peptide was assembled as above on resin (0.1 mmol based on initial resin loading) incorporating the desired Fmoc-protected O-allylated serine. The resin was washed successively with DMF, DCM and ether after acetylation. The resin was dried under reduced pressure and taken up in an anhydrous DCM solution of Grubbs I catalyst (4 mL, 4 mg/mL, 0.02 mmol) After 18 h, the reaction was filtered and the resin was washed with DCM. The olefin metathesis step was repeated twice in order to fully consume starting material. The resin was taken up in 10% EDT/DMF (4 mL) and agitated at ambient temperature for 18 h. The resin was filtered and washed successively with NMP, DCM and ether. The cyclized peptide was simultaneously cleaved from the resin and the protecting groups on the sidechains removed by treating the resin with a solution (7.5 mL) of trifluoroacetic acid (TFA) (93.5%), water (2.5%), triisopropylsilane (TIPS), (2.5%), and ethanedithiol (EDT) (2.5%). The mixture was filtered and to the filtrate was added chilled diethylether (40 mL). The mixture was centrifuged and the supernatant decanted. The pellet was suspended in 1:1 acetonitrile/water (5 mL) and lyophilized. The crude peptide was purified using C₁₈ reversed-phase HPLC with acetonitrile and water (with 0.1% TFA) as the mobile phase. The fractions containing the desired peptide were pooled. The fractions were lyophilized twice in 50:50 acetonitrile:HCl (aq) (60 mN, then 10 mN) and once in 50:50 acetonitrile:water to give two isomers of SP-41 as a colorless solid; ealier eluting isomer (5.4 mg), later eluting isomer (5.7 mg). MS (ESI) m/z, found 875.43 (M+3H/3). calcd. 874.89 (M+3H/3).

SP-28 (R=—CH₃): The peptide was synthesized in the same manner as SP-27 to yield two isomers of SP-28 as a colorless solid; ealier eluting isomer (5.5 mg), later eluting isomer (4.4 mg). MS (ESI) m/z, found 884.04 (M+3H/3). calcd. 884.13 (M+3H/3).

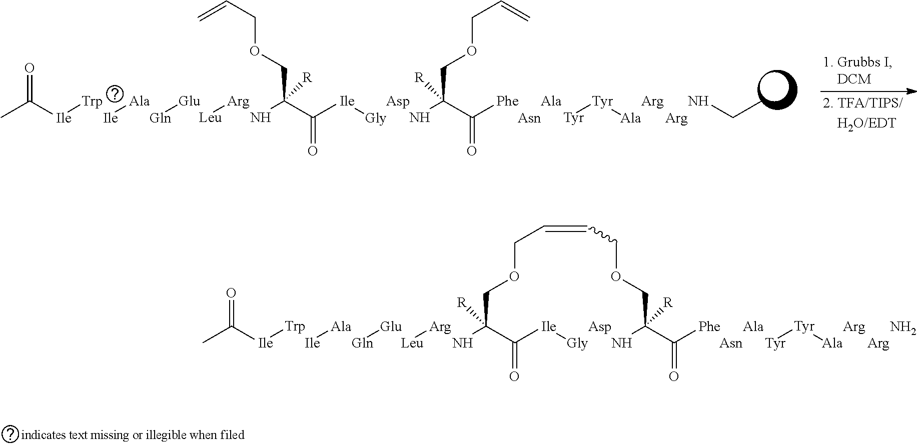

(SEQ ID NOS 151 and 118, respectively)

SP-38 (R=—H): The peptide was synthesized in the same manner as SP-27 to yield SP-38 as a colorless solid (12.9 mg). MS (ESI) m/z, found 805.82 (M+3H/3). calcd. 805.45 (M+3H/3).

SP-39 (R=—CH₃): The peptide was synthesized in the same manner as SP-27 to yield SP-39 as a colorless solid (7.2 mg). MS (ESI) m/z, found 815.42 (M+3H/3). calcd. 814.79 (M+3H/3).

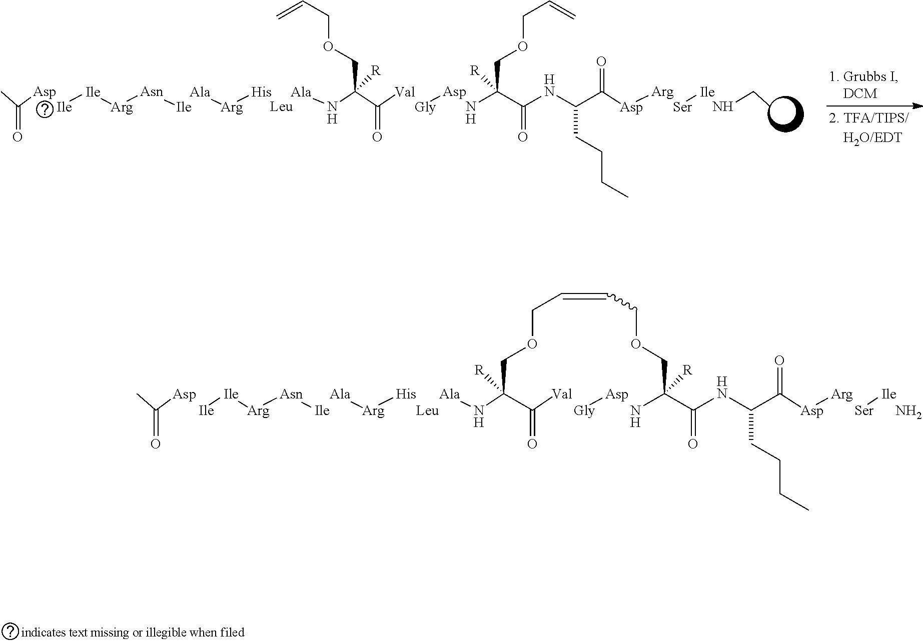

(SEQ ID NOS 152 and 130, respectively)

### Example 5

### Cell Viability Assays of Tumor Cell Lines Treated with Peptidomimetic Macrocycles of the Invention

Tumor cell lines are grown in specific serum-supplemented media (growth media) as recommended by ATCC and the NCI. A day prior to the initiation of the study, cells were plated at optimal cell density (15,000 to 25,000 cells/well) in 200 μl growth media in microtiter plates. The next day, cells were washed twice in serum-free/phenol red-free RPMI complete media (assay buffer) and a final volume of 100 μl assay buffer was added to each well. Human peripheral blood lymphocytes (hPBLs) were isolated from Buffy coats (San Diego Blood Bank) using Ficoll-Paque gradient separation and plated on the day of the experiment at 25,000 cells/well.

Peptidomimetic macrocycles were diluted from 1 mM stocks (100% DMSO) in sterile water to prepare 400 μM working solutions. The macrocycles and controls were then diluted 10 or 40 fold or alternatively serially two-fold diluted in assay buffer in dosing plates to provide concentrations of either 40 and 20 μM or between 1.2 and 40 μM, respectively. 100 μL of each dilution was then added to the appropriate wells of the test plate to achieve final concentrations of the polypeptides equal to 20 or 5 μM, or between 0.6 to 20 μM, respectively. Controls included wells without polypeptides containing the same concentration of DMSO as the wells containing the macrocycles, wells containing 0.1% Triton X-100, wells containing a chemo cocktail comprised of 1 μM Velcade, 100 μM Etoposide and 20 μM Taxol and wells containing no cells. Plates were incubated for 4 hours at 37° C. in humidified 5% CO₂ atmosphere.

Towards the end of the 4 hour incubation time, 22 μl FBS was added to each well for a total concentration of 10% FBS. After addition of serum, the plates were incubated for an additional 44 hours at 37° C. in humidified 5% CO₂ atmosphere. At the end of the incubation period, MTT assay was performed according to manufacturer's instructions (Sigma, catalog #M2128) and absorbance was measured at 560 nm using Dynex Opsys MR Plate reader.

### Example 6

### Melting Temperature (Tₘ) Determination

Lyophilized peptidomimetic macrocycle is dissolved in ddH₂O to a final concentration of 50 μM.

Tm is determined by measuring the circular dichroism (CD) spectra in a Jasco-810 spectropolarimeter at a fixed wavelength of 222 nm between the temperatures of 5-95° C. The following parameters are used for the measurement: data pitch, 0.1° C.; bandwidth, 1 nm and path length, 0.1 cm averaging the signal for 16 seconds.

### Example 7

### Sample Preparation for Plasma Stability Determination

For ex-vivo plasma stability studies 10 μM of peptidomimetic macrocycles are incubated with pre-cleared human and mouse plasma at 37° C. for 0, 15 and 120 minutes. At the end of each incubation time, 100 μL of sample is removed, placed in a fresh low retention eppendorf tube with 300 μl of ice cold MEOH. The samples are centrifuged at 10,000 rpm, the supernatant removed and placed in a fresh low retention eppendorf tube and 200 μl of HPLC H₂O was added to each sample. Samples are then analyzed by LC-MS/MS as indicated below.

### Example 8

### Protease Stability Assays

For pepsin testing, each pair consisting of α-methyl and α,α-methyl di-substituted peptidomimetic macrocycle sequences was combined (5 μM each) with positive control linear peptide (5 μM) in a safflower oil/ethanol/water suspension, 0.2:9.8:90, v/v (%), buffered (pH 1.8) with 0.015M HCl and 0.15 M NaCl. Eleven pairs were tested in eleven working solutions, each of which was aliquoted into 5×0.5 ml reaction volumes for pepsin incubation times of 10, 30, 45, 60 min, and a 0 min control with no pepsin added that was incubated for 60 min. The reaction was initiated at 38-40° C. by adding 20 μl of pepsin-silica gel slurry (0.4 μg pepsin) and shaking vials continually during subsequent incubation in 40° C. oven. At each time point, the reaction was stopped by addition of 500 μl of 48:48:2 v/v (%) hexafluoro-2-propanol/acetonitrile/TFA. A biphasic mixture formed after mixing and the bottom layer liquid was subsequently injected in duplicate for LC/MS analyses in MRM detection mode. The reaction rate for each peptide was calculated in Excel as (−1) times the slope derived by a linear fit of the natural logarithm of un-calibrated MRM response versus enzyme incubation time. The reaction half-life for each peptide was calculated as ln 2/rate constant.

A similar procedure was used for trypsin testing. Each pair consisting of α-methyl and α,α-methyl di-substituted peptidomimetic macrocycle sequences was combined (5 μM each) with linear peptide (5 μM) in a safflower oil/ethanol/water suspension, 0.2:9.8:90, v/v (%), buffered (pH7.8) with 0.055 M Tris-acetate, 0.15M NaCl. Ten pairs were tested in ten working solutions, each of which was aliquoted into 5×0.5 ml reaction volumes for trypsin incubation times of 10, 20, 30, 60 min, and a 0 min —no trypsin added control that was incubated for 60 min. The reaction was initiated at 38-40° C. by adding 20 μl of trypsin-silica gel slurry (0.4 μg or 0.32 μg trypsin) and shaking vials continually during subsequent incubation in 40° C. oven. At each time point, the reaction was stopped by addition of 500 μl of 48:48:2 v/v (%) hexafluoro-2-propanol/acetonitrile/TFA. A biphasic mixture formed after mixing and the bottom layer liquid was subsequently injected in duplicate for LC/MS analyses in MRM detection mode. The reaction rate for each peptide was calculated in Excel as (−1) times the slope derived by a linear fit of the natural logarithm of un-calibrated MRM response versus enzyme incubation time. The reaction half-life for each peptide was calculated as ln 2/rate constant.

Control mixtures (no protease added) appeared stable (>60 min) in buffers containing safflower oil/ethanol/water suspension, 0.2:9.8:90, v/v (%), buffered with 0.015M HCl and containing 0.15M NaCl.

### Example 9

### Cellular Penetrability Assays by FACS Intracellular Detection of FITC/FAM-Labeled Peptidomimetic Macrocycles

Jurkat cells or SJSA-1 cells were cultured with RPMI-1640 (Gibco, Cat#72400) plus 10% FBS (Gibco, Cat#16140) and 1% Penicillin+Streptomycin (Hyclone, Cat#30010) at 37° C. in a humidified 5% CO₂ atmosphere. Jurkat cells were split at 1×10⁶/ml cell density, or SJSA-1 cells were seeded at 2×10⁵/ml/well in 24 well plates a day prior to the initiation of the study. The next day, cells were washed twice in Opti-MEM media (Gibco, Cat#51985) with spinning at 1200 rpm, 23° C. for 5 min. The Jurkat cells were seeded in 0.9 ml of Opti-MEM in absence of serum at density of 1×10⁶ cells in 24 well plates. The SJSA-1 cells were fed with 0.9 ml of Opti-MEM in absence of serum in each well. Peptides were diluted to 2 mM stock in DMSO, followed by dilution to 400 μM in sterile water; further dilution to 100 μM was done using OPTI-MEM; same dilutions were made for DMSO controls. Thus 100 μl of 100 μM peptide working solution or final diluted DMSO were then added into appropriate wells to achieve peptide final concentration of 10 μM and the DMSO concentration 0.5% in 1 ml volume. Plates were incubated at 37° C. incubator with 5% CO₂, or 4° C. on wet ice for 1 hour or 4 hours. At the end of each time point, the cell suspension were diluted with RPMI-1640 plus 10% FBS and washed twice with 1×PBS (Gibco) plus 0.5% BSA and subjected to 0.25% Trypsin-EDTA (Gibco, Cat#25200) for 15 min at 37° C. Cells were then washed with 1 ml of RPMI-1640 plus 10% FBS and twice with 0.5 ml of 1×PBS plus 0.5% BSA (Sigma, Cat#A7906), spinning at 4000 rpm, 4° C. for 5 min (Eppendorf Centrifuge 5415D). Cells were suspended in 0.5 ml of 1×PBS plus 0.5% BSA. The Fluorescence or FAM intensity was measured by FACSCalibur, (BD Biosciences). FACS data were analyzed with Flowjo software (BD Biosciences), and the data were graphed with Prism software. All assays were performed in duplicate.

### Example 10

### Intravenous Pharmacokinetic Analysis

The IV dose formulation is prepared by dissolving peptidomimetic macrocycles in 5% DMSO/D5W to achieve a 10 mg/Kg/dose. Canulated CrI:CD® (SD) male rats (7-8 weeks old, Charles River Laboratories) are used in these studies. Intravenous doses are administered via the femoral cannula and the animals are dosed at 10 mL/kg per single injection. Blood for pharmacokinetic analysis is collected at 10 time points (0.0833, 0.25, 0.5, 1, 2, 4, 6, 8, 12 and 24 hrs post-dose). Animals are terminated (without necropsy) following their final sample collection.

The whole blood samples are centrifuged (˜1500×g) for 10 min at ˜4° C. Plasma is prepared and transferred within 30 min of blood collection/centrifugation to fresh tubes that are frozen and stored in the dark at ˜−70° C. until they are prepared for LC-MS/MS analysis.

Sample extraction is achieved by adding 10 μL of 50% formic acid to 100 μL plasma (samples or stds), following by vortexing for 10 seconds. 500 μL acetonitrile is added to the followed by vortexing for 2 minutes and centrifuged at 14,000 rpm for 10 minutes at −4° C. Supernatants are transferred to clean tubes and evaporated on turbovap <10 psi at 37° C. Prior to LC-MS/MS analysis samples are reconstituted with 100 μL of 50:50 acetonitrile:water.

The peak plasma concentration (Cₘₐₓ), the time required to achieve the peak plasma concentration (tₘₐₓ), the plasma terminal half-life (t_{1/2}), the area under the plasma concentration time curve (AUC), the clearance and volume of distribution are calculated from the plasma concentration data. All pharmacokinetic calculations are done using WinNonlin version 4.1 (Pharsight Corp) by non-compartmental analysis.

The following LC-MS/MS method is used. In brief, the LC-MS/MS instruments used was an API 365 (Applied Biosystems). The analytical column was a Phenomenex Synergi (4μ, Polar-RP, 50 mm×2 mm) and mobile phases A (0.1% formic acid in water) and B (0.1% formic acid in methanol) are pumped at a flow rate of 0.4 ml/min to achieve the following gradient:

| Time (min) | % B |
|---|---|
| 0 | 15 |
| 0.5 | 15 |
| 1.5 | 95 |
| 4.5 | 95 |
| 4.6 | 15 |
| 8.0 | Stop |

MRM: 814.0 to 374.2 (positive ionization)

### Example 11

### Mass Spectroscopy-Based Assays for Receptor Binding Assay

Protein-Ligand Binding Experiments for Bcl-x_{L}.

Simple protein-ligand binding experiments were conducted using the following representative procedure outlined for a simple system-wide control experiment using 1 μM SP-4 and 5 μM Bcl-x_{L}. A 1 μL DMSO aliquot of a 40 μM stock solution of SP-4 is dissolved in 19 μL of PBS (Phosphate-buffered saline: 50 mM, pH 7.5 Phosphate buffer containing 150 mM NaCl). The resulting solution is mixed by repeated pipetting and clarified by centrifugation at 10 000 g for 10 min. To a 4 μL aliquot of the resulting supernatant is added 4 μL of 10 μM BCL-x_{L} in PBS. Each 8.0 μL experimental sample thus contains 40 pmol (1.5 μg) of protein at 5.0 μM concentration in PBS plus 1 μM SP-4 and 2.5% DMSO. Duplicate samples thus prepared for each concentration point are incubated for 60 min at room temperature, and then chilled to 4° C. prior to size-exclusion chromatography-LC-MS analysis of 5.0 μL injections. Samples containing a target protein, protein-ligand complexes, and unbound compounds are injected onto an SEC column, where the complexes are separated from non-binding component by a rapid SEC step. The SEC column eluate is monitored using UV detectors to confirm that the early-eluting protein fraction, which elutes in the void volume of the SEC column, is well resolved from unbound components that are retained on the column. After the peak containing the protein and protein-ligand complexes elutes from the primary UV detector, it enters a sample loop where it is excised from the flow stream of the SEC stage and transferred directly to the LC-MS via a valving mechanism. The (M+3H)³⁺ ion of SP-4 is observed by ESI-MS at m/z 883.8, confirming the detection of the protein-ligand complex.

Example Protein-Ligand Kd Titration Experiments for Bcl-xL.

Protein-ligand K_{d} titations experiments were conducted as follows: 2 μL DMSO aliquots of a serially diluted stock solution of titrant peptidomimetic macrocycle (5, 2.5, . . . , 0.098 mM) are prepared then dissolved in 38 μL of PBS. The resulting solutions are mixed by repeated pipetting and clarified by centrifugation at 10 000 g for 10 min. To 4.0 μL aliquots of the resulting supernatants is added 4.0 μL of 10 μM BCL-x_{L} in PBS. Each 8.0 μL experimental sample thus contains 40 pmol (1.5 μg) of protein at 5.0 μM concentration in PBS, varying concentrations (125, 62.5, . . . , 0.24 μM) of the titrant peptide, and 2.5% DMSO. Duplicate samples thus prepared for each concentration point are incubated at room temperature for 30 min, then chilled to 4° C. prior to SEC-LC-MS analysis of 2.0 μL injections. The (M+H)¹⁺, (M+2H)²⁺, (M+3H)³⁺, and/or (M+Na)¹⁺ ion is observed by ESI-MS; extracted ion chromatograms are quantified, then fit to equations described in Annis et al, 2007, to derive the binding affinity K_{d}. Similar assays were performed for Mcl-1, and Bcl-2.

Competitive Binding Experiments for Bcl-x_{L}.

A mixture ligands at 40 μM per component is prepared by combining 2 μL aliquots of 400 μM stocks of each of the three compounds with 14 μL of DMSO. Then, 1 μL aliquots of this 40 μM per component mixture are combined with 1 μL DMSO aliquots of a serially diluted stock solution of titrant peptide (10, 5, 2.5, . . . , 0.078 mM). These 2 μL samples are dissolved in 38 μL of PBS. The resulting solutions are mixed by repeated pipetting and clarified by centrifugation at 10 000 g for 10 min. To 4.0 μL aliquots of the resulting supernatants is added 4.0 μL of 10 μM BCL-x_{L} in PBS. Each 8.0 μL experimental sample thus contains 40 pmol (1.5 μg) of protein at 5.0 μM concentration in PBS plus 0.5 μM ligand, 2.5% DMSO, and varying concentrations (125, 62.5, . . . , 1.95 μM) of the titrant peptide. Duplicate samples thus prepared for each concentration point are incubated at room temperature for 60 min, then chilled to 4° C. prior to SEC-LC-MS analysis of 2.0 μL injections. The (M+H)¹⁺, (M+2H)²⁺, (M+3H)³⁺, and/or (M+Na)¹⁺ ion for the titrant and each mixture component is observed by ESI-MS; extracted ion chromatograms then analyzed as described in Annis et al, 2004, to rank-order binding affinities of the mixture components. More detailed information on these and other methods is available in “A General Technique to Rank Protein-Ligand Binding Affinities and Determine Allosteric vs. Direct Binding Site Competition in Compound Mixtures.” Annis, D. A.; Nazef, N.; Chuang, C. C.; Scott, M. P.; Nash, H. M. J. Am. Chem. Soc. 2004, 126, 15495-15503 and “ALIS: An Affinity Selection-Mass Spectrometry System for the Discovery and Characterization of Protein-Ligand Interactions” D. A. Annis, C.-C. Chuang, and N. Nazef. In Mass Spectrometry in Medicinal Chemistry. Edited by Wanner K, Hofner G: Wiley-VCH; 2007:121-184. Mannhold R, Kubinyi H, Folkers G (Series Editors): Methods and Principles in Medicinal Chemistry.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of improving a biological activity of a polypeptide comprising the step of providing a crosslinked alpha-helical polypeptide comprising a crosslinker wherein a hydrogen atom attached to an α-carbon atom of an amino acid of said crosslinked polypeptide is replaced with a substituent of formula R-, wherein:
R- is alkyl, alkenyl, alkynyl, arylalkyl, cycloalkylalkyl, heteroalkyl, or heterocycloalkyl, unsubstituted or substituted with halo-; and
the biological activity of said polypeptide is improved at least 2-fold relative to a corresponding polypeptide lacking said substituent.

2. The method of claim 1, wherein the biological activity of said polypeptide is improved on average at least 2-fold.

3-5. (canceled)

6. The method of claim 1, wherein the crosslinker connects two α-carbon atoms.

7. The method of claim 1, wherein two α-carbon atoms are substituted with independent substituents of formula R-.

8. The method of claim 1, wherein one α-carbon atom to which the crosslinker is attached is substituted with a substituent of formula R-.

9-41. (canceled)

42. A method for preparing a cross-linked polypeptide comprising:
a) providing a precursor polypeptide comprising at least two moieties capable of undergoing reaction to form a covalent bond between said two moieties, wherein at least one of said moieties is attached to an α-carbon atom of an amino acid of said crosslinked polypeptide, and wherein at least two isomers may be obtained following said reaction;
b) replacing a hydrogen atom attached to said α-carbon atom with a substituent of formula R-, wherein R- is alkyl, alkenyl, alkynyl, arylalkyl, cycloalkylalkyl, heteroalkyl, or heterocycloalkyl, unsubstituted or substituted with halo-; and
c) incubating said precursor polypeptide in conditions that promote formation of at least one crosslink between said moieties, wherein one of said at least two isomers is obtained in a greater yield than another of said at least two isomers.

43. The method of claim 42, wherein the ratio of said at least two isomers obtained is greater than 2:1.

44-45. (canceled)

46. The method of claim 42, wherein the crosslinked polypeptide is alpha-helical.

47. The method of claim 42, wherein the crosslinker connects two α-carbon atoms.

48. The method of claim 42, wherein two α-carbon atoms are substituted with independent substituents of formula R-.

49. The method of claim 42, wherein one α-carbon atom to which the crosslinker is attached is substituted with a substituent of formula R-.

50-65. (canceled)

66. A peptidomimetic macrocycle comprising an amino acid sequence which is at least 60% identical to an amino acid sequence of any one of SEQ ID NOs. 125, 126, or 143-44.

67. A peptidomimetic macrocycle comprising an amino acid sequence which is at least 60% identical to an amino acid sequence of any one of the sequences in Table 1, 2, 3, or 4.

68. A method of determining the binding and affinity of peptidomimetic macrocycles or peptidomimetic precursors to acceptor proteins comprising attaching a fluorescent tracer onto said peptidomimetic macrocycles or peptidomimetic precursors, exciting said fluorescent tracer with polarized light, and measuring polarization with a spectrophotometer.

69. The method of claim 68, wherein said spectrophotometer is a luminescence spectrophotometer.

70. The method of claim 68, wherein said tracer is FITC.
